(19) United States
(12) Patent Application Publication
(10) Pub. No.: US 2005/0170372 A1
(43) Pub. Date: Aug. 4, 2005

(54) Methods and systems for profiling biological systems

(76) Inventors: Noubar B. Afeyan, Lexington, MA (US); Jan van der Greef, Driebergen-Rijsenburg (NL); Frederick E. Regnier, W. Lafayette, IN (US); Aram S. Adourian, Woburn, MA (US); Eric K. Neumann, Lexington, MA (US); Matej Oresic, Espoo (FI); Elwin Robbert Verheij, Zeist (NL)

Correspondence Address: KIRKPATRICK & LOCKHART NICHOLSON GRAHAM LLP;(FORMERLY KIRKPATRICK & LOCKHART LLP), 75 STATE STREET, BOSTON, MA, 02109-1808, US

(21) Appl. No.: 10/922,820

(22) Filed: Aug. 20, 2004

Related U.S. Application Data
(63) Continuation-in-part of application No. 10/218,880, filed on Aug. 13, 2002.
(60) Provisional application No. 60/496,657, filed on Aug. 20, 2003.
(60) Provisional application No. 60/312,145, filed on Aug. 13, 2001.

(51) Int. Cl. C12Q 1/68; G06F 19/00; G01N 33/48; G01N 33/50; G01N 33/00

(52) U.S. Cl. 435/6; 436/86; 702/19

(57) ABSTRACT

Methods and systems are disclosed for developing profiles of a state of a biological system based on the discernment of similarities, differences, and/or correlations between a plurality of data sets that are derived from one or more biomolecular component types, one or more biological sample types, and/or one or more types of measurements.

This application claims priority to and the benefit of U.S. Provisional Patent Application Ser. No. 60/496,657, filed on Aug. 20, 2003, and is a continuation-in-part of U.S. patent application Ser. No. 10/218,880, filed on Aug. 13, 2002, which claims priority to and the benefit of U.S. Provisional Patent Application Ser. No. 60/312,145, filed on Aug. 13, 2001, the entire disclosures of which are incorporated by reference herein.

### FIELD OF THE INVENTION

The invention relates to the field of data processing and evaluation. More particularly, the invention relates to methods and systems for profiling a state of a biological system, e.g., a mammal such as a human.

### BACKGROUND

Current approaches to understanding biology, such as genomics and proteomics, typically focus on a single aspect of a biological system at any one time. The “omics” technology revolution, particularly that of genomics, has provided a basis for studies of a single type of biomolecule both in single cell organisms, e.g., yeast, and in simple, multi-cellular systems, such as sea urchin embryos. In both types of studies, the systems are perturbed by environmental changes and/or genetic manipulation to enable the correlation of gene expression changes in a number of different scenarios. Construction of in silico interaction networks is facilitated by looking at interdependencies between and among genes from several different perspectives. However, while modern quantitative genomic technologies are readily available, the resulting information may be of low precision and utility. For example, in one sea urchin study, a perturbation was deemed significant only if it gave rise to a three-fold or greater change in gene expression. Although a number of experimental factors might contribute to the net variability in a system and reduce precision, a significant biological effect may be manifested by a change that occurs well under a three-fold cut-off.

Analyzing and understanding a complex, multi-cellular organism, such as a mammal, is much more complicated. When studying the state of a complex biological system, one must take into account the multi-compartmental character of the system, not to mention the variety of cell and tissue types that will have unique gene expression and protein and metabolite levels. Current studies that rely on the analysis of a single aspect of a biological system, e.g., a single type of molecule or target, usually are not robust enough to understand the entire biological system or subsystem that may be involved in a particular molecular pathway or disease.

An important challenge in the understanding of a biological system of a mammal and the development of new drugs for complex, multi-factorial diseases is the identification and validation of biomarkers/surrogate markers. Moreover, it appears that instead of single biomarkers being indicative of a state of a biological system, biomarker patterns or biomarker sets may be necessary to characterize and diagnose homeostasis or disease states for a biological system, where multiple levels of the biological system are simultaneously considered in the analysis. Accordingly, there is a need for methods and systems that consider a biological system as a whole and that are able to advance the study of human disease, and the discovery and development of pharmaceutical products.

### SUMMARY OF THE INVENTION

The applicants of this patent application are pioneers in a field known as “systems biology.” In contrast to analysis of an individual aspect of a biological system, systems biology is the study of biology as an integrated biological system including genetic, protein and metabolic components, and their pathways, which are in flux and interdependent. Rather than artificially simplifying the inherent complexity of biological processes that underlie the biology of a complex organism, e.g., the biological processes involved in human diseases or that govern drug responses, the methods and systems described herein embrace the complexities and interdependencies contained within a biological system. By appropriately visualizing and considering the complexity of a biological system, a skilled artisan can undertake biological research at the systems level, developing a profile for a state of a biological system which provides insight into the biological system as a whole.

The application describes methods and systems to analyze complex clinical samples of mammals including humans at a biological systems level to provide new information about the state of a biological system that was previously unobtainable through traditional chemistries or genomics alone. Using the methods and systems described herein, it is possible to gain insight into biological pathways and mechanisms of disease and drug response. More specifically, the methods and systems can analyze and integrate data at the biomolecular component type level, i.e., the gene/gene transcript, protein and metabolite level, to create knowledge that advances pharmaceutical research and development by providing new insights into the molecular mechanisms of health and disease, which further the development and discovery of novel therapeutics to treat human disease.

To develop a profile of a state of a biological system, e.g., a disease state, multiple measurements on complex biological samples are performed. Subsequently, comprehensive gene, gene transcript, protein, and/or metabolite profiling coupled with correlation analysis and network modeling provides insight into a biological system at a systems level so that connections, correlations, and relationships among thousands of diverse, measurable molecular components can be achieved. Such knowledge then may be used directly for the development of therapeutic agents or biomarkers, may be used in combination with clinical information, and/or may serve as a basis for directed, hypothesis-driven experiments designed to further elucidate pathophysiologic mechanisms. Further, tracking changes of a profile of a biological system can improve many aspects of pharmaceutical discovery and development, including drug safety and efficacy, drug response, and the etiology of disease.

The application addresses limitations in current profiling techniques by providing a method and system, or a “technology platform,” having the ability to integrate a plurality of data sets, which may include two or more biomolecular component types, to elucidate information conveying associations between or among components or networks of interactions among components. The methods and systems utilize statistical analyses of a plurality of data sets, e.g., spectrometric data, to develop a profile of a state of a biological system, e.g., a mammal such as a human. The data sets comprise multiple measurements of the biological system and are derived from three primary sources: a biological sample type, a measurement technique, and a biomolecular component type. The application further describes a technology platform that facilitates the discernment of similarities, differences, and/or correlations not only within a single biomolecular component type within a sample or biological system, but also across two or more biomolecular component types.

In a broad aspect, a method of profiling a state of a biological system includes evaluating with statistical analysis a plurality of data sets of a biological system and comparing features among the plurality of data sets to determine one or more sets of differences among at least portion of the plurality of data sets. The action of comparing the features among the plurality of data sets can include direct comparison of one feature in a first data set to a corresponding feature in another data set. The action of comparing the features also can include correlating or associating features between or among data sets such as correlations associated with and/or resulting from the statistical analysis, e.g., multivariate analysis. Based on the results of the evaluation and comparison, a profile for a state of the biological system can be developed.

Another method of profiling a state of a biological system in a mammal includes evaluating with statistical analysis a plurality of data sets for a biomolecular component type and comparing features among the plurality of data sets to determine one or more sets of differences among at least a portion of the plurality of data sets; evaluating with statistical analysis a plurality of data sets for another biomolecular component type and comparing features among the plurality of data sets to determine one or more sets of differences among at least a portion of the plurality of data sets; and correlating the results of the above described analyses to develop a profile for a state of the biological system.

A further method of profiling a state of a biological system in a mammal includes evaluating with statistical analysis a plurality of data sets comprising measurements from at least two biomolecular component types and comparing features among the plurality of data sets to determine one or more sets of differences among at least a portion of the plurality of data sets; and developing a profile for a state of the biological system based on the results of the above-described analysis.

Central to the methods and systems described herein is the analysis of a plurality of data sets. The plurality of data sets include measurements derived from more than one biological sample type, more than one type of measurement technique, more than one biomolecular component type, or a combination of at least two of a biological sample type, a measurement technique, and a biomolecular component type. The biological system preferably is in a mammal, such as a human. A biomolecular component type includes a protein, a glycoprotein, a gene, a gene transcript, and a metabolite.

A biological sample type includes, among others, blood, plasma, serum, cerebrospinal fluid, bile, saliva, synovial fluid, pleural fluid, pericardial fluid, peritoneal fluid, sweat, feces, nasal fluid, ocular fluid, intracellular fluid, intercellular fluid, lymph, urine, liver cells, epithelial cells, endothelial cells, kidney cells, prostate cells, blood cells, lung cells, brain cells, skin cells, adipose cells, tumor cells, and mammary cells. Data sets can include measurements from one biological sample type that is treated differently, or from one biological sample type that is collected or analyzed at different times.

A measurement technique includes, among others, liquid chromatography, gas chromatography, high performance liquid chromatography, capillary electrophoresis, mass spectrometry, liquid chromatography-mass spectrometry, gas chromatography-mass spectrometry, high performance liquid chromatography-mass spectrometry, capillary electrophoresis-mass spectrometry, nuclear magnetic resonance spectrometry, parallel hybridization assay, parallel sandwich assay, and competitive assay. Data sets can include measurements from different instrument configurations of a single type of measurement technique.

Subsequent to developing a profile for the state of a biological system, the profile can be compared to a profile of another state of a biological system, where the biological systems are the same or different. A profile also can be compared to a database of profiles to evaluate whether the state of the biological system matches or is similar to a known state. The methods described herein may be carried out by an article of manufacture having a computer-readable medium with computer-readable instructions embodied thereon for performing the methods.

Other aspects and advantages of the invention will become apparent from the following figures, detailed description, and claims, all of which illustrate the principles of the invention by way of example only.

### BRIEF DESCRIPTION OF THE FIGURES

The foregoing and other objects, features, and advantages of the invention described above will be more fully understood from the following description of various illustrative embodiments, when read together with the accompanying drawings. In the drawings, like reference characters generally refer to the same parts throughout the different views. The drawings are not necessarily to scale, and emphasis instead is generally placed upon illustrating the principles of the invention.

FIG. 1 is a schematic flow diagram illustrating the integration of genomic, proteomic, metabolomic and clinical data sets to develop a profile of a biological system.

FIG. 2 is a flow diagram of various analytical and processing steps as applied to a plurality of data sets according to an illustrative embodiment of the invention.

FIG. 3 illustrates the experimental design of the ApoE3-Leiden transgenic mouse gene expression experiment.

FIG. 4 illustrates a significance plot for the gene expression experiment.

FIG. 5 illustrates a significance plot for the selected 1059 peptide peaks from four liver fractions.

FIG. 6 illustrates a block design for the synthetic data GIST experiment.

FIG. 7 illustrates scatter plots and a normal probability plot for variety 1 of the synthetic GIST data set.

FIG. 8 illustrates scatter plots and a normal probability plot for variety 2 of the synthetic GIST data set.

FIG. 9 illustrates scatter plots and a normal probability plot for variety 3 of the synthetic GIST data set.

FIG. 10 illustrates a significance plot for the synthetic GIST data set.

FIG. 11 illustrates a flow diagram that describes the treatment of the gene expression data derived from a biological sample.

FIG. 12 illustrates a flow diagram that describes the treatment of the protein data derived from a biological sample.

FIG. 13 illustrates a flow diagram that describes the treatment of the metabolite data derived from a biological sample.

FIG. 14 illustrates a flow diagram that describes the integration of a plurality of data sets derived from two or more biomolecular component types.

FIG. 15 illustrates a gene expression analysis that reveals mRNA abundance.

FIG. 16 illustrates results for selected groups from a gene expression analysis.

FIG. 17 illustrates results for selected groups from a gene expression analysis.

FIG. 18 illustrates intensity plots of LC/MS total ion chromatograms of proteins from plasma samples.

FIG. 19 illustrates total ion chromatograms from LC/MS profiling of proteins from plasma samples.

FIG. 20 illustrates LC/MS chromatograms acquired from the digested liver proteins of five transgenic and five wildtype mice.

FIG. 21 illustrates ¹H NMR spectra of metabolites extracted from plasma from transgenic and wildtype mice.

FIG. 22 illustrates mass chromatograms of plasma lipids recorded using LC/MS for transgenic and wildtype mice.

FIG. 23 illustrates individual gene, protein, and metabolite spectra that are normalized and then concatenated to form a single factor spectrum for comparison across individual biomolecular component types.

FIG. 24 illustrates clustering of wildtype and transgenic mice data resulting from Principal Component and Discriminant (“PC-DA”) statistical analysis.

FIG. 25 illustrates a difference factor spectrum of peptides exhibiting significant differences (note m/z value 1366).

FIG. 26 illustrates a mass spectrum and a sequence of a peptide (m/z value 1366) from mouse plasma recorded using LC/MS/MS, where the peptide deduced from the MS/MS spectrum is identified as residues 57-79 in the sequence of human apolipoprotein E3.

FIG. 27 illustrates a correlation network between biomolecular component types.

FIG. 28 illustrates a map of known relations between the correlation network associations and published information.

FIG. 29 illustrates typical “offerings” or “deliverables,” in terms of biomarkers (“Markers”) or therapeutic agents that can be derived from a systems biology analysis.

FIG. 30A illustrates the experimental design of the ApoE3-Leiden transgenic mouse experiment.

FIG. 30B illustrates a scatter plot of the cDNA microarray data.

FIG. 31A illustrates the LC/MS chromatograms for the digested liver protein fraction for the ten samples.

FIG. 31B illustrates the clustering analysis of the tryptic peptide profiles.

FIG. 31C illustrates a factor spectrum of the liver protein data.

FIG. 32A illustrates the clustering resulting from the principal component analysis of the liver lipid data set.

FIG. 32B illustrates a factor spectrum of the liver lipid data set.

FIGS. 33A, 33B, and 33C illustrate a comprehensive systems analysis based on data from three biomolecular component types, where a relative abundance of 1.0 is 100%. (FIG. 33A—mRNA; FIG. 33B—protein; FIG. 33C—lipid).

FIG. 34 is a schematic illustrating hyperlipidemia and atherosclerosis in a blood vessel.

FIG. 35 illustrates a whole plasma parallel proteo-metabolic profiling scheme.

FIG. 36 illustrates NMR spectra for a wildtype mouse plasma sample (WT) and a transgenic mouse plasma sample (TG).

FIG. 37 illustrates a PC-DA score plot showing clustering of NMR data for the transgenic mouse, represented by triangles, and the wildtype (or control) mouse, represented by circles.

FIG. 38 illustrates a difference spectrum characterized by a number of lines representing various metabolic components.

FIG. 39 illustrates total ion chromatograms (TIC's) for deproteinated lipid fractions from transgenic (TG) mice and wildtype (WT) mice analyzed by a 4-step gradient in the LC dimension with mass spectrum acquired over 200-1700 m/z mass range.

FIG. 40 illustrates total ion chromatograms from transgenic (TG) mice and wildtype (WT) mice protein fractions obtained from tryptic peptides.

FIG. 41 illustrates a score plot showing PC-DA clusters for the wildtype (WT) and transgenic mouse (TG).

FIG. 42 illustrates difference factor spectra for protein and metabolite components.

FIG. 43 illustrates a schematic representation of data analysis workflow.

FIG. 44 illustrates the workflow for an unsupervised clustering analysis for multiple platforms.

FIG. 44A illustrates COSA unsupervised clustering of LC/MS proteomic data, revealing four distinct clusters.

FIG. 44B illustrates COSA unsupervised clustering of multiple data sets that have been concatenated.

FIG. 45 illustrates the workflow for selecting and comparing components of one sample that are different from another sample.

FIG. 45A illustrates a representative graph of selected protein, lipid, and metabolite differences between rat groups identified using the univariate statistical method.

FIG. 46 illustrates a correlation network for the comparison between drug-treated diseased rodents and vehicle-treated diseased rodents (drug effect on disease).

FIG. 47 illustrates an intensity plot visualization of correlations between pairs of components in the drug-treated diseased rodents and vehicle-treated diseased rodents (drug effect on disease).

FIG. 48 illustrates a plot showing ratios between groups based on the means of the peak intensity values within each group (after normalization and scaling) related to peptides from certain proteins.

FIG. 49 illustrates COSA distance clustering using human LC/MS lipid peaks.

FIG. 50 illustrates the workflow for a comparison and correlation of human sample data with non-human sample data.

FIG. 50A illustrates the results of a COSA analysis of human serum samples in which the input data set used for classification consisted of 366 lipid peaks chosen from the rodent model of the human disease.

FIG. 51 illustrates the success rate of an SVM linear classifier as a function of number of lipid peaks.

FIG. 52 illustrates a comparison of lipid abundance changes and correlations across human and rodent species.

FIG. 53 illustrates the workflow for analysis of several data sets.

FIG. 54 illustrates a graphical representation of selecting analytes for a biomarker.

FIG. 55 illustrates the performance of a fifteen analyte biomarker in grouping samples.

FIG. 56 illustrates the list of analytes from FIG. 55.

### DETAILED DESCRIPTION OF THE INVENTION

The methods and systems disclosed herein rely on multiple measurements of biological samples, including analysis of metabolites, proteins, genes and gene transcripts, to permit a skilled artisan to understand a biological system in greater depth than an approach that examines only one of these factors. Understanding the biological system as a whole can improve multiple aspects of pharmaceutical discovery and development, including drug safety and efficacy, drug response, and the etiology of disease. As described herein, a systems biology platform can integrate genomics, proteomics and metabolomics, and bioinformatics, and results in a data integration and knowledge management platform that generates connections, correlations, and relationships among thousands of measurable molecular components to develop of a profile of a state of a biological system. Resulting profiles can be combined with clinical information to increase the knowledge of a state of a biological system.

A “profile” of a biological system is a summary or analysis of data representing distinctive features or characteristics of the biological system, e.g., of a mammal such as a human. The data can include measurements or features derived from a biological sample type, a type of measurement technique, and a biomolecular component type. The data often are spectral or chromatographic features that are in the form of a graph, table, or some similar data compilation. A profile typically is a set of data features that permit characterization of a state of a biological system.

A profile can be considered to include one or more “biomarkers” of a biological system. A biomarker generally refers to a biological component type, e.g., a gene, a gene transcript, a protein or a metabolite, whose qualitative and/or quantitative presence or absence in a biological system is an indicator of a biological state of an mammal. Thus, a profile can be considered to be a set of distinctive biomarkers, e.g., spectral or chromatographic features, that permit characterization of a state of a biological system. A profile also can be considered to include correlations and other results of analyses of the data sets, e.g., causality. Thus, a profile can comprise a plurality of different elements as described above, or can comprise only one of these elements, e.g., biomarker(s).

A “state of a biological system” refers to a condition in which the biological system exists, either naturally or after a perturbation. Examples of a state of a biological system include, but are not limited to, a normal or healthy state, a disease state, a pharmacological agent response, a toxicological state, a biochemical regulation (e.g., apoptosis), an age response, an environmental response, and a stress response. The biological system preferably is in a mammal, which includes humans and non-human mammals such as mice, rats, guinea pigs, dogs, cats, monkeys, and the like.

A profile of a state of a biological system permits the comparison of one profile to another profile to determine whether the profiles are in the same state, e.g., a healthy or a diseased state. A biological system is better characterized using a multivariate analysis rather than using multiple measurements of the same variable because multivariate analysis envisions the biological system as a whole. Disparate data from multiple, different sources is treated as if in a single dimension rather than in multiple dimensions. Consequently, the analysis of data is more informative and typically provides a profile that is more robust and predictive than one that is developed by systematically evaluating multiple components individually or relies on one particular biomolecular component type.

A “biomolecular component type” refers to a class of biomolecules generally associated with a level of a biological system. For example, genes and gene transcripts (which may be interchangeably referred to herein) are examples of biomolecular component types that generally are associated with gene expression in a biological system, and where the level of the biological system is referred to as genomics or functional genomics. Proteins and their constituent peptides (which may be interchangeably referred to herein), are another example of a biomolecular component type that generally is associated with protein expression and modification, and where the level of the biological system is referred to as proteomics. Glycoproteins also are considered a biomolecular component type. Another example of a biomolecular component type is metabolites (which also may be referred to as small molecules), which generally are associated with a level of a biological system referred to as metabolomics. Metabolites include, but are not limited to, lipids, steroids, amino acids, organic acids, bile acids, eicosanoids, neuropeptides, vitamins, neurotransmitters, carbohydrates, ionic organics, nucleotides, inorganics, xenobiotics, peptides, trace elements, and pharmacophore and drug breakdown products.

The methods described herein may be used to develop a profile of a state of a biological system based on any single biomolecular component type as well as based on two or more biomolecular component types. Profiles of biomolecular component types facilitate the development of comprehensive profiles of different levels of a biological system, e.g., genome profiles, transcriptomic profiles, proteome profiles and metabolome profiles, and permit their integration and analysis. That is, the methods may be used to analyze measurements derived from one or more biological sample type, one or more type of measurement technique, or a combination of at least one each of a biological sample type and a measurement technique so as to permit the evaluation of similarities, differences, and/or correlations in a single biomolecular component type or across two or more biomolecular component types. From these measurements, better insight into underlying biological mechanisms may be gained, novel biomarkers/surrogate markers may be detected, and intervention routes may be developed.

A “biological sample type” includes, but is not limited to, blood, blood plasma, blood serum, cerebrospinal fluid, bile acid, saliva, synovial fluid, pleural fluid, pericardial fluid, peritoneal fluid, sweat, feces, nasal fluid, ocular fluid, intracellular fluid, intercellular fluid, lymph urine, tissue, liver cells, epithelial cells, endothelial cells, kidney cells, prostate cells, blood cells, lung cells, brain cells, adipose cells, tumor cells, and mammary cells. The sources of biological sample types may be different subjects; the same subject at different times; the same subject in different states, e.g., prior to drug treatment and after drug treatment; different sexes; different species, e.g., a human and a non-human mammal; and various other permutations. Further, a biological sample type may be treated differently prior to evaluation such as using different work-up protocols.

A “measurement technique” refers to any analytical technique that generates or provides data that is useful in the analysis of a state of a biological system. For example, measurement techniques include, but are not limited to, mass spectrometry (“MS”), nuclear magnetic resonance spectroscopy (“NMR”), liquid chromatography (“LC”), gas-chromatography (“GC”), high performance liquid chromatography (“HPLC”), capillary electrophoresis (“CE”), gel electrophoresis (“GE”) and any known form of hyphenated mass spectrometry in low or high resolution mode, such as LC/MS, GC/MS, CE/MS, MS/MS, MSⁿ, and other variants. Measurement techniques include biological imaging such as magnetic resonance imagery (“MRI”), video signals, and an array of fluorescence, e.g., light intensity and/or color from points in space, and other high throughput or highly parallel data collection techniques.

Measurement techniques also include optical spectroscopy, digital imagery, oligonucleotide array hybridization, protein array hybridization, DNA hybridization arrays (“gene chips”), immunohistochemical analysis, polymerase chain reaction, nucleic acid hybridization, electrocardiography, computed axial tomography, positron emission tomography, and subjective analyses such as found in text-based clinical data reports. For a particular analysis, different measurement techniques may include different instrument configurations or settings relating to the same measurement technique.

A “measurement” refers to an element of a data set that is generated by a measurement technique. A “data set” includes measurements derived from a one or more sources. For example, a data set derived from a measurement technique includes a series of measurements collected by the same technique, i.e., a collection or set of data of related measurements. Further, data sets more broadly may represent collections of diverse data, e.g., protein expression data, gene expression data, metabolite concentration data, magnetic resonance imaging data, electrocardiogram data, genotype data, single nucleotide polymorphism data, and other biological data. That is, any measurable or quantifiable aspect of a biological system being studied may serve as the basis for generating a given data set.

A “feature” of a data set refers to a particular measurement associated with that data set that may be compared to another data set. For example, a profile typically is a set of data features that permit characterization of a state of a biological system.

Data sets may refer to substantially all or a sub-set of the data associated with one or more measurement techniques. For example, the data associated with the spectrometric measurements of different sample sources may be grouped into different data sets. As a result, a first data set may refer to experimental group sample measurements and a second data set may refer to control group sample measurements. In addition, data sets may refer to data grouped based on any other classification considered relevant. For example, data associated with the spectrometric measurements of a single sample source may be grouped into different data sets based on the instrument used to perform the measurement, the time a sample was taken, the appearance of a sample, or other identifiable variables and characteristics.

Accordingly, one data set may include a sub-set of another data set. For example, a grouping based on appearance of the sample may include one or more experimental group data sets. Where the measurement technique is NMR, a data set may include one or more NMR spectra. Where the measurement technique is ultraviolet (UV) spectroscopy, a data set may include one or more UV emission or absorption spectra. Similarly, where the measurement technique is MS, a data set may include one or more mass spectra. Where the measurement technique is a chromatographic-MS technique, like LC/MS or GC/MS, a data set may include one or more mass chromatograms. Alternatively, a data set of a chromatographic-MS technique may include one or more total ion current (“TIC”) chromatograms or reconstructed TIC chromatograms. In addition, it should be realized that the term “data set” includes both raw spectrometric data and data that has been preprocessed, e.g., to remove noise, to correct a baseline, to smooth the data, to detect peaks, and/or to normalize the data.

“Spectrometric data” refers to any data that may be represented in the form of a graph, table, vector, array or some similar data compilation, and may include data from any spectrometric or chromatographic technique. The term “spectrometric measurement” includes measurements made by any spectrometric or chromatographic technique.

Central to the methods disclosed herein is the statistical analysis of a plurality of data sets. “Statistical analysis” includes parametric analysis, non-parametric analysis, univariate analysis, multivariate analysis, linear analysis, non-linear analysis, and other statistical methods known to those skilled in the art. Multivariate analysis, which determines patterns in apparently chaotic data, includes, but is not limited to, principal component analysis (“PCA”), discriminant analysis (“DA”), PCA-DA, canonical correlation (“CC”), cluster analysis, partial least squares (“PLS”), predictive linear discriminant analysis (“PLDA”), neural networks, and pattern recognition techniques.

Of course before performing multivariate analysis, the raw data may be preprocessed to assist in the comparison of different data sets. In particular, to compare data across different biomolecular component types, appropriate preprocessing should be performed. Preprocessing of the data may include (i) aligning data points between data sets, e.g., using partial linear fit techniques to align peaks of spectra of different samples; (ii) normalizing the data of the data sets, e.g., using standards in each measurement to adjust peak height; (iii) reducing the noise and/or detecting peaks, e.g., setting a threshold level for peaks so as to discern the actual presence of a species from potential baseline noise; and/or (iv) other data processing techniques known in the art. Data preprocessing can include entropy-based peak detection as disclosed in U.S. Pat. No. 6,743,364, and partial linear fit techniques (such as found in J. T. W. E. Vogels et al., “Partial Linear Fit: A New NMR Spectroscopy Processing Tool for Pattern Recognition Applications,” Journal of Chemometrics, vol. 10, pp. 425-38 (1996)).

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps.

It should be understood that the order of steps or order for performing certain actions is immaterial so long as the invention remains operable, i.e., a profile of a biological system is developed. Moreover, two or more steps or actions may be conducted simultaneously.

The methods described herein generally include evaluating with statistical analysis a plurality of data sets of a biological systems and comparing features among the data sets to determine one or more sets of differences among at least a portion of the data sets so as to develop a profile for a state of a biological system based on the comparison. In some embodiments, the data sets are derived from one or more biological sample types and include measurements derived from one or more measurement techniques. In other embodiments, the data sets are derived from two or more biological sample types and include one or more different types of spectrometric measurements of a sample of the biological system.

In certain embodiments, the data sets are preprocessed and evaluated using multivariate analysis. In other embodiments, more than one statistical analysis is performed on the plurality of data sets, on various permutations of the plurality of data sets, and/or on the results of a particular statistical analysis. For example, a profile may be developed by separately evaluating a plurality of data sets including measurements derived from proteins in the biological system and a plurality of data sets including measurements derived from metabolites in the biological system, then evaluating with statistical analysis the results of the individual analyses to develop a profile for the biological system that includes both proteins and metabolites. Alternatively, the plurality of data sets relating to proteins and metabolites of the biological systems may be simultaneously evaluated with statistical analysis.

Analogously, a profile can be developed from data sets including measurements derived from a protein and a gene; a protein and a gene transcript; a gene and a gene transcript; a gene and a metabolite; and a gene transcript and a metabolite. A profile also can be developed from data sets including measurements derived from a protein, a gene and a gene transcript; a protein, a gene and a metabolite; a protein, a gene transcript and a metabolite; and a gene, a gene transcript and a metabolite; and a protein, a gene, a gene transcript and a metabolite. In addition, each of the above permutations can include, in addition or as a substitution, a glycoprotein.

Measurements for a particular biomolecular component type usually are generated by a measurement technique or techniques that are often used and known in the art for that particular biomolecular component type. For example, an analysis of metabolites may use NMR, e.g., ¹H-NMR; LC/MS; GC/MS; and MS/MS. Analysis of other biomolecular component types may use LC/MS; GC/MS; and MS/MS.

In one embodiment, the method generally includes selecting a biological sample; preparing the biological sample based on the biochemical components to be investigated and the spectrometric techniques to be employed; measuring the components in the biological samples using spectrometric and chromatographic techniques; measuring selected molecule subclasses using NMR and MS-approaches to study compounds; preprocessing the raw data; using statistical analysis, which will be described in more detail below, to analyze the preprocessed data to identify patterns in measurements of single subclasses of molecules or in measurements of components using NMR or MS; and using statistical analysis to combine data sets from distinct experiments and identify patterns of interest in the data.

The technology platform may also include normalizing a plurality of data sets to facilitate comparison of the data across biomolecular component types. The invention also provides techniques for determining associations/correlations between biomolecular component types of suitable data sets using linear, non-linear or other mathematical tools. Moreover, using these associations and/or correlations to postulate networks of interacting biomolecular components to determine causality among these associations, and to establish hypotheses about the biological processes underlying the observations which give rise to the data sets, is still another aspect of the methods and systems described herein.

The application also provides an article of manufacture where the functionality of a method disclosed herein is embedded on a computer-readable medium such as, but not limited to, a floppy disk, a hard disk, an optical disk, a magnetic tape, a PROM, an EPROM, CD-ROM, or DVD-ROM. The functionality of the method may be embedded on the computer-readable medium in any number of computer-readable instructions or languages such as FORTRAN, PASCAL, C, C++, BASIC and assembly language. Further, the computer-readable instructions may be written in a script, macro, or functionally embedded in commercially available software such as EXCEL or VISUAL BASIC. In other aspects, the application provides systems adapted to practice the methods described herein.

The data processing device may include an analog and/or digital circuit adapted to implement the functionality of one or more of the methods disclosed herein using at least in part information provided by the spectrometric instrument. In some embodiments, the data processing device may implement the functionality of the methods described herein as software on a general-purpose computer. In addition, such a program may set aside portions of a computer's random access memory to provide control logic that affects the spectrometric measurement acquisition, statistical analysis of data sets, and/or profile development for a biological system. In such an embodiment, the program may be written in any one of a number of high-level languages, such as FORTRAN, PASCAL, C, C++, or BASIC. Further, the program can be written in a script, macro, or functionality embedded in proprietary software or commercially available software, such as EXCEL or VISUAL BASIC. Additionally, the software could be implemented in an assembly language directed to a microprocessor resident on a computer. For example, the software can be implemented in Intel 80×86 assembly language if it is configured to run on an IBM PC or PC clone. The software may be embedded on an article of manufacture including, but not limited to, a computer-readable program medium such as a floppy disk, a hard disk, an optical disk, a magnetic tape, a PROM, an EPROM, or CD-ROM.

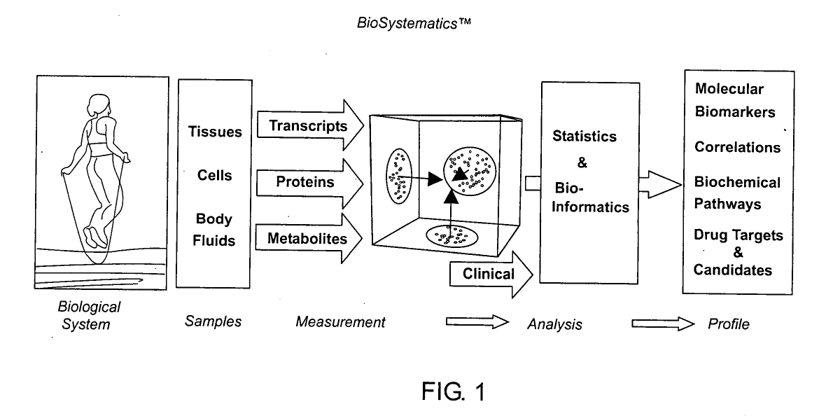

As shown in FIG. 1, in some embodiments, the method begins with parallel analyses of gene transcripts (mRNA), protein, and metabolite quantitative profiles derived from complex samples extracted from both diseased and healthy populations. The mean quantities, as well as the ranges and variances, for all measured compounds are collectively analyzed using methods such as pattern recognition to identify molecules to link gene response, protein activity, and metabolite dynamics. The methods disclosed herein, coined BioSystematics™, then can be employed to translate covariant sets of genes including gene transcripts, proteins, and metabolites, optionally with clinical information, into an understanding of their biochemical interaction to elucidate a profile of a biological system and target information. This information, the extent to which particular groups of molecules co-vary, and existing pathway knowledge then are used to assemble molecular networks and place compounds in their biological context so as to develop a profile of a state of the biological system.

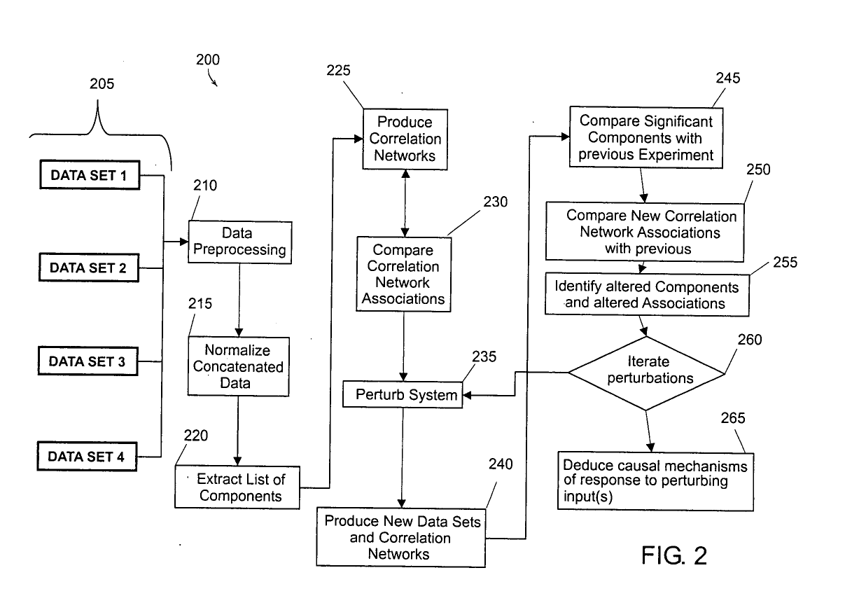

FIG. 2 shows a flow chart of one embodiment of an analytical method 200. It should be understood that one or more of the steps described below can be omitted and/or the order of steps can be changed so long as the embodiment remains operable, i.e., capable of developing a profile of a state of a biological system. One or more data sets 205 taken from two or more biomolecular component types are subjected to an initial preprocessing step 210 prior to further data analysis. In a preferred embodiment, the initial processing step typically includes concatenating one or more of the plurality of data sets. This initial preprocessing step may also include integrating together the data sets based on a suitable schema or data hierarchy. In some embodiments, the initial processing step includes both a concatenation step and an integration step. The initial processing optionally may include, follow, or precede various forms of preprocessing including, but not limited to, data smoothing, noise reduction, baseline correction, and peak detection.

The data sets that are the subject of the initial preprocessing step may include any measurable or quantifiable aspect of the biological system being studied. For example, the data sets may represent collections of, e.g., protein expression data, gene expression data, metabolite concentration data, magnetic resonance imaging data, electrocardiogram data, genotype data, and/or single nucleotide polymorphism data. Statistical methods such as principal component analysis may be utilized to convert the data sets to factor spectra, which are simply a processed form of the raw data.

Means for comparing data sets of completely unrelated phenomena with disparate units of measure is necessary, especially given the broad range of data sets that may be employed. Referring to FIG. 2, for such disparate data sets, a normalization step 215, which is described in more detail below, may be implemented. Generally, individual data sets are normalized by scaling the data set with optimal scaling parameters calculated using a maximum likelihood estimator. Normalization facilitates comparison of data sets taken from one or more biomolecular component types.

An extraction step 220 is typically performed on the processed data. In the extraction step, one or more list(s) of components, which exhibit statistically significant changes, are extracted. The components typically are biological component types, or more specifically biomolecular component types. Further, these changes also are quantified as part of the extraction step. The extraction step typically involves a statistical analysis to discern the differences and/or similarities between the data sets. The extraction step and associated quantification of differences facilitates discerning similarities, differences, and/or correlations between or among two or more biomolecular component types for the biological sample under investigation.

Suitable forms of statistical analysis appropriate for quantifying the change between component types include, e.g., principal component analysis (“PCA”), discriminant analysis (“DA”), PCA-DA, canonical correlation (“CC”), partial least squares (“PLS”), predictive linear discriminant analysis (“PLDA”), neural networks, and pattern recognition techniques. In one embodiment, PCA-DA is performed at a first level of correlation that produces a score plot, i.e., a plot of the data in terms of two principal components. Subsequently, the same or a different statistical analysis is performed on the data sets based on the differences and/or similarities discerned from previous analysis.

For example, in one embodiment, where a processed data set includes a PCA-DA score plot, the next level of statistical processing may be a loading plot produced by a PCA-DA analysis. This second level of correlation bears a hierarchical relationship to the first level in that loading plots provide information on the contributions of individual input vectors to the PCA-DA that in turn are used to produce a score plot. For example, where each data set includes a plurality of mass chromatograms, a point on a score plot represents mass chromatograms originating from one sample source. In comparison, a point on a loading plot represents the contribution of a particular mass or range of masses to the correlations between data sets. Similarly, where each data set includes a plurality of NMR spectra, a point on a score plot represents one NMR spectrum. In comparison, a point on the corresponding loading plot represents the contribution of a particular NMR chemical shift value or range of values to the correlations between data sets.

FIG. 2 also depicts a correlation network production step 225, which follows the extraction step 220. The formulation of the correlation networks indicates potential associations among the extracted list of components developed previously by the preceding step. A correlation network is a representation (graphical, mathematical, or otherwise) of the biomolecular component types of a system that vary in abundance between one or more groups of samples. Two components are “correlated” if they vary in a somewhat synchronous manner. For example, if both a gene and a protein are upregulated in group 1 as compared to group 2 and the upregulation is consistent across all the biological samples including group 1, then the gene and protein are considered to be “correlated.” Analogously, biomolecular component types also may be anti-correlated. Moreover, different “strengths of correlation” exist, which depend on how tightly synchronous the relationship is between or among the two or more biomolecular types.

A comparison step 230 is performed after the correlation networks have been established. The correlation network associations, which encompass both correlations and anti-correlations, are compared and evaluated based on existing knowledge of the component or biological system under investigation. This knowledge relates to the associations which may be ascertained from established sources such as research literature and/or experimental studies.

Subsequently, a perturbation step 235 typically is performed as part of the larger analysis. The biological system subject to investigation is typically perturbed by changing an experimental parameter and monitoring the system for a prescribed amount of time. Examples of perturbations include, but are not limited to, introducing a drug, altering a gene, changing an environmental condition, or making another suitable change. A perturbation also encompasses the idea of comparing across species, i.e., performing the workflow on an animal system and performing substantially the same workflow on a human system to investigate the similarities and/or differences between or among species.

Following the perturbation step 235, new data sets and correlation networks are produced 240. Thus, as a result of the perturbations introduced into a given biological system or sample, new data sets arise that are measurable. Similarly, as part of step 240, new correlation networks may be developed based on those novel post-perturbation data sets. The statistically significant changes in the new data sets, as determined in comparison to the pre-perturbation data sets, are discerned by comparing the statistically significant biological component types in the new data sets with the component types of the previous experimental results 245. In addition to looking at the statistical changes between biomolecular component types before and after system perturbation 245, correlation networks may be analyzed in kind. Therefore, the correlation network association networks may be compared before and after perturbation 250. After these two levels of comparison 245, 250 have been performed, alterations or changes between components and associations can be identified 255.

Thereafter, perturbations to the system being investigated can be iterated 260. A feedback loop results among the initial perturbations to the system, the system itself, the production of new data sets, the comparison of significant components with the previous experiment, the comparison of new correlation network associations with previous associations, and the identification of changes. The feedback loop may be iterated until causal relations can be identified 265 between multiple biomolecular component types and the correlation and networks which characterize their impact on the biological system.

Referring back to the normalization step 215 in FIG. 2 and introduced above, a method for normalizing gene expression data, protein data, and metabolite level data is now described. A sample variety effect, an array effect, and a dye effect are introduced into a log-linear model, and a maximum likelihood maximization technique is applied to calculate all the parameters of the model and determine the optimal scaling factor for each array and dye. The normalization method is generic and can be applied to a variety of data, experimental setups, and designs. The model described below uses terminology from gene expression analysis. For example, the “array” in proteomics experiment could be one mass spectrometer run, and the “dye” could describe all samples used during the single run. Nevertheless, other biomolecular component types could be analyzed using the model described below.

Normalization model. The data matrix x is characterized by the gene index g(g=1 . . . N_{g}), array index i(i=1 . . . Nᵢ), dye index k(k=1 . . . Nₖ), and the variety index v(v=1 . . . Nᵥ). For each variety v, there are Cᵥ samples corresponding to it, so Nₛₐₘₚₗₑₛ=ΣᵥCᵥ=NᵢNₖ. Since variety assignment is a function of array and dye indices, each data point is uniquely described by indices g, i, and k. For convenience the matrix is transformed logarithmically:
y_{gik}=log(x_{gik}). (1)
Data is described by the following model:
y_{gik}=μ_{gv}+Aᵢ+Dₖ+ε_{gik}, (2)
where the gene and variety effects are described by μ_{gv}, the array effect by Aᵢ, the dye effect by Dₖ, and the error function by ε_{gik}. The error function is assumed to be normally distributed with zero mean and the variance σ_{gv}², i.e., the variance is permitted to be different for each gene and variety. The variety index v is a unique function of i and k, and can be written as {i, k}∈v. Since the gene and variety, array, and dye effects are assumed to be fixed, the distribution of expression levels can be described as:

$$P ( {y}_{gik} \vert {μ}_{g v} , {A}_{i} , {D}_{k} , {σ}_{g v}^{2} ) = \frac{1}{\sqrt{2 {πσ}_{g v}^{2}}} exp ( - \frac{{( {y}_{gik} - {μ}_{g v} - {A}_{i} - {D}_{k} )}^{2}}{2 {σ}_{g v}^{2}} ) . \qquad ( 3 )$$

A maximum likelihood estimation is used to calculate the optimal scaling parameters used to properly normalize the data. Solving for the parameters μ_{gv}, Aᵢ, Dₖ, and σ_{gv} leads to the following equations:

$$\begin{matrix}{{u}^{⋒}}_{gv} = \frac{1}{{C}_{v}} {\sum }_{i k ∈ v} ( {y}_{gik} - {{A}^{⋒}}_{i} - {{D}^{⋒}}_{k} ) , \\ {{A}^{⋒}}_{i} = \frac{1}{{N}_{i}} {\sum }_{g k} ( {y}_{gik} - {{μ}^{⋒}}_{g v} - {{D}^{⋒}}_{k} ) , \\ {{D}^{⋒}}_{k} = \frac{1}{{N}_{i}} {\sum }_{g i} ( {y}_{gik} - {{μ}^{⋒}}_{g v} - {{A}^{⋒}}_{i} ) , \\ {{σ}^{⋒}}^{2} = \frac{1}{{N}_{g} {N}_{i} {N}_{k}} {\sum }_{i k ∈ v} {( {y}_{gik} - {{μ}^{⋒}}_{g v} - {{A}^{⋒}}_{i} - {{D}^{⋒}}_{k} )}^{2} .\end{matrix} \qquad ( 4 )$$

The optimal scaling factors for each array and dye are then:
sᵢₖ=−Aᵢ−Dₖ, (5)
so the normalized expression levels are:
\tilde{x}_{gik}=x_{gik}×exp(sᵢₖ). (6)

Significance tests and bootstrap methods. The normalized data may be compared to a null model, and a p-value may be calculated that measures the probability that the deviation of the data from the null model can be attributed to the random error. The parameter used for comparison is the fold ratio between the two chosen varieties. To evaluate the method, a t-test is performed to compare the two chosen varieties. [Sheskin, Handbook of Parametric and Nonparametric Procedures, Chapman & Hall/CRC, Boca Raton, Fla. (2000).] The corresponding p-values were calculated for each gene. When assessing the statistical significance of fold change for each gene, one needs to take into consideration the total N_{g} p-values calculated, as several p-values with p<1/N_{g} are expected. To account for this, the overall likelihood, P(p), of observing a p-value ≦p for any of the N_{g} genes is used. Assuming independence of all genes, the overall likelihood is estimated with:
P(p)≈1−(1−p)^{Ng}. (7)

Assuming independence of genes is obviously an oversimplification, and the correct way to calculate p-values and P(p) values is by using the bootstrap method with the parameters (μ_{gv}, Aᵢ, Dₖ, σ_{gv}) of the null model being used to general random data sets.

### EXAMPLE 1

### Normalization of Gene Expression Data from the Liver of an APOE*3-Leiden Transgenic Mouse

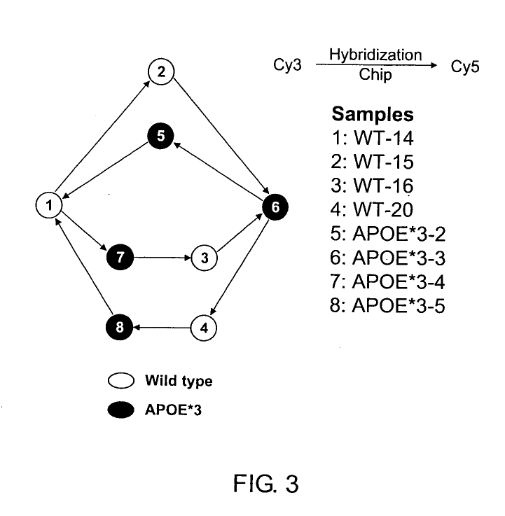

To illustrate the normalization method, a study of the ApoE3-Leiden transgenic mouse was performed. A total of 9,596 genes were analyzed using ten cDNA microarrays. Samples were collected from a total of four ApoE3-Leiden transgenic (TG) mice and four wild type (WT) mice. An optimized design of the experiment is shown in FIG. 3. The variety vector was therefore
Vars=[1 1 1 2 2 1 1 2 2 1 1 2 2 1 1 2 2 2 2 1]. (8)

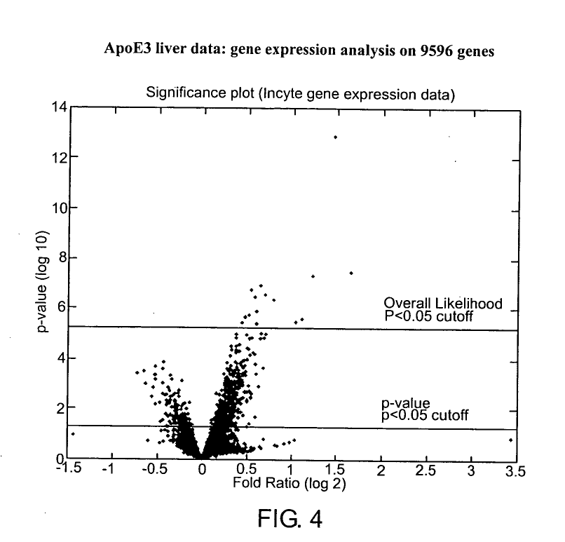

A t-test was applied, comparing the normalized values of transgenic and wild type mice. FIG. 4 shows the significance plot of the data based on p-values from the t-test and fold ratios. The horizontal line on top shows the overall likelihood P(p)=0.05 cutoff, while the lower line shows the cutoff, p=0.05. Only 16 genes satisfy the most stringent former criterion, while there are 713 genes in the p<0.05 range.

Protein data from liver. Eight samples from eight different animals, four transgenic and four wildtype, were analyzed in eight experiments. The variety vector is therefore:
Vars=[1 1 1 1 2 2 2 2]. (9)

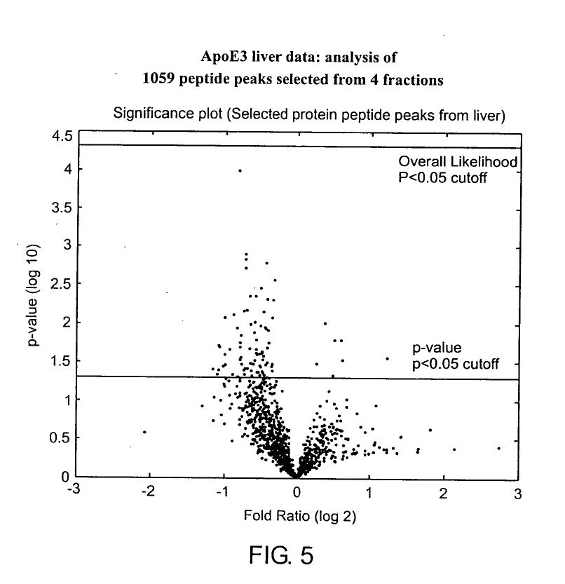

Mass spectrometry (MS) spectra were selected from a total of four fractions, each containing 1600 peaks. The MS spectra were processed using the IMPRESS algorithm, which was developed at the University of Leiden and is described in U.S. Pat. No. 6,743,364. IMPRESS peak characterization software uses an information theoretic measure (IQ) to determine peak significance (between 0 and 1). A peak in the data set with IQ>0.5 was retained for a majority of the samples (i.e., 5 or more out of 8). A total of 1059 peaks were selected, 5 from fraction 1, 271 in fraction 3, 454 in fraction 4, and 329 in fraction 5. The significance plot is shown in FIG. 5. There are no peaks satisfying the P(p)=0.05 cutoff criterion, while there are 84 peaks with p<0.05. In this case, more data are necessary to determine if normalization should be performed on different fractions separately.

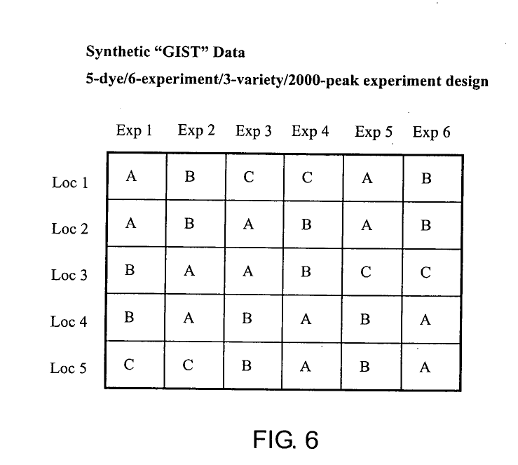

Synthetic “GIST” data. To perform a test of the normalization method on data with higher number of dyes, an experiment on synthetic data with 2000 peaks, 5 dyes, 3 varieties, and 6 experiments was performed. This could potentially correspond to proteomics experiments performed using the Global Internal Standards Technology (“GIST”) [Chakraborty, A. and Regnier, F., J. Chromatog. A 949, 173-84 (2002)] The experiment design is shown in FIG. 6 and can also be described by the following variety vector:
Vars=[1 1 2 2 3 2 2 1 1 3 3 1 1 2 2 3 2 2 1 1 1 1 3 2 2 2 2 3 1 1]. (10)

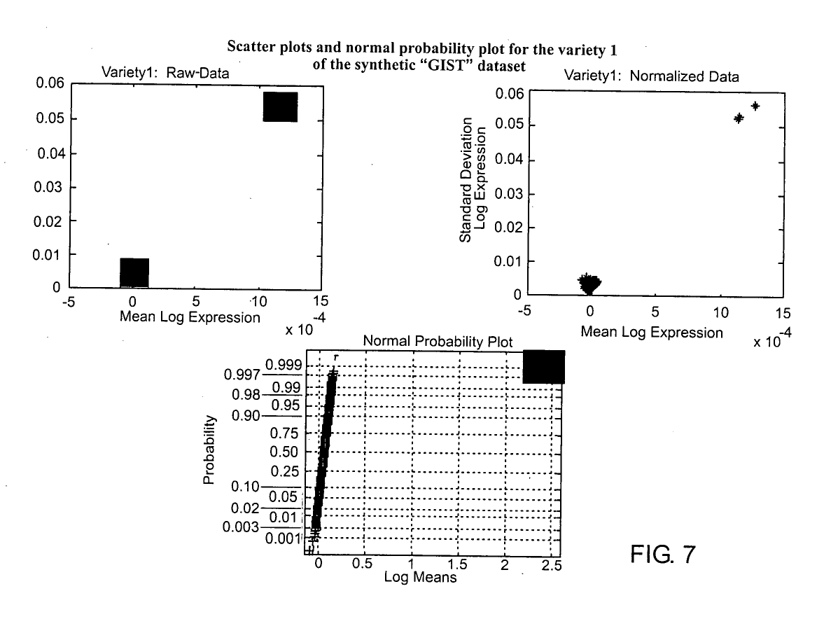

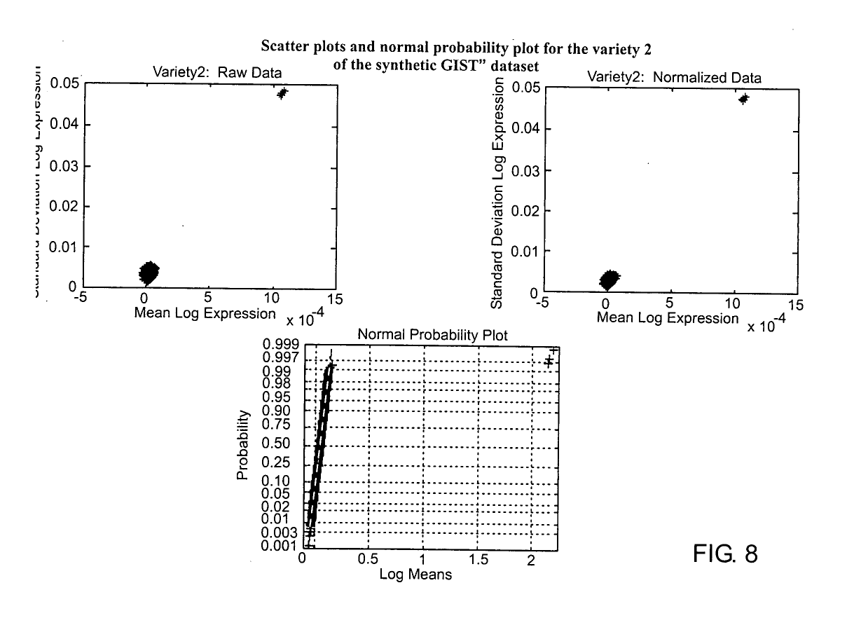

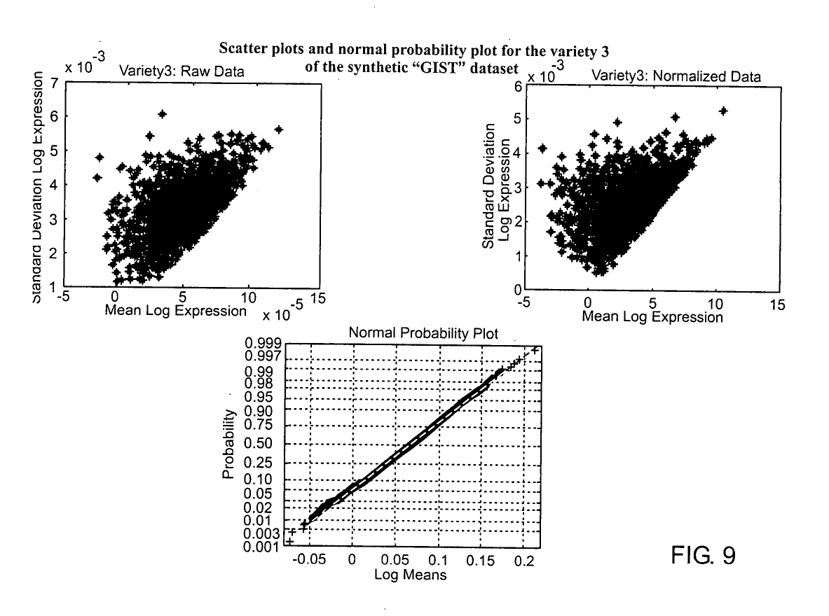

The background for each peak has been selected using Gaussian random number generator, set to equal mean and variance. Three large peaks have then been added for each of the variety 1 and 2, respectively, while variety 3 has been kept as control. FIGS. 7-9 show the scatterplots and normal probability plots for each of the varieties. The three outliers are clearly seen for varieties 1 and 2. The fold ratio:

$$Fold = \frac{〈 Variety2 〉}{〈 Variety1 〉} , \qquad ( 11 )$$

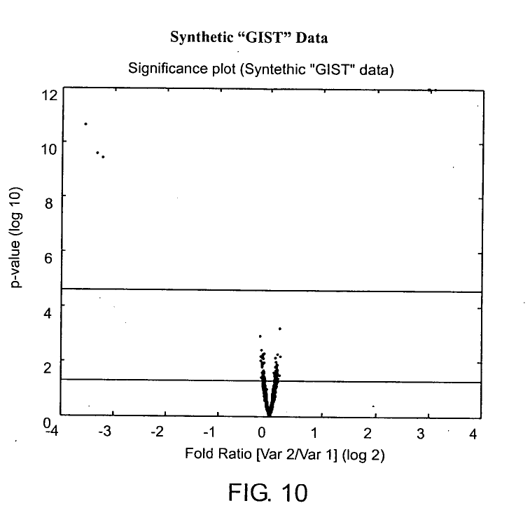

was calculated for each peak, and a t-test was used to compare the two varieties. The significance plot is shown in FIG. 10. As expected, only six outliers satisfy the P(p)=0.05 cutoff criterion, while there are a total of 94 peaks satisfying p<0.05, despite the fact each peak (except the six outliers) has been generated randomly for each sample independently.

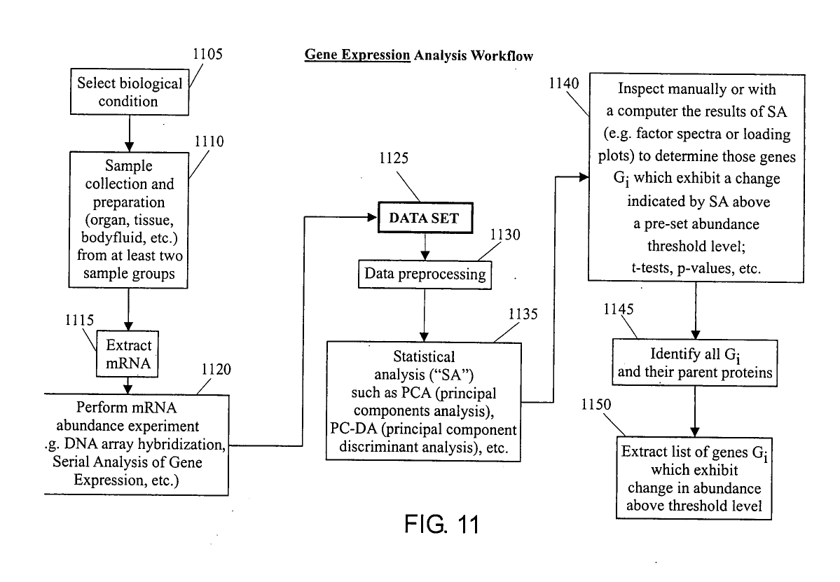

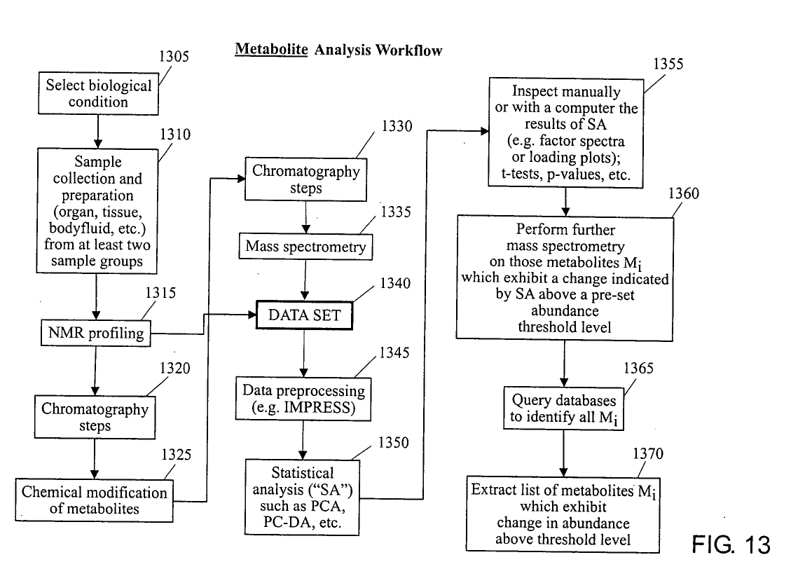

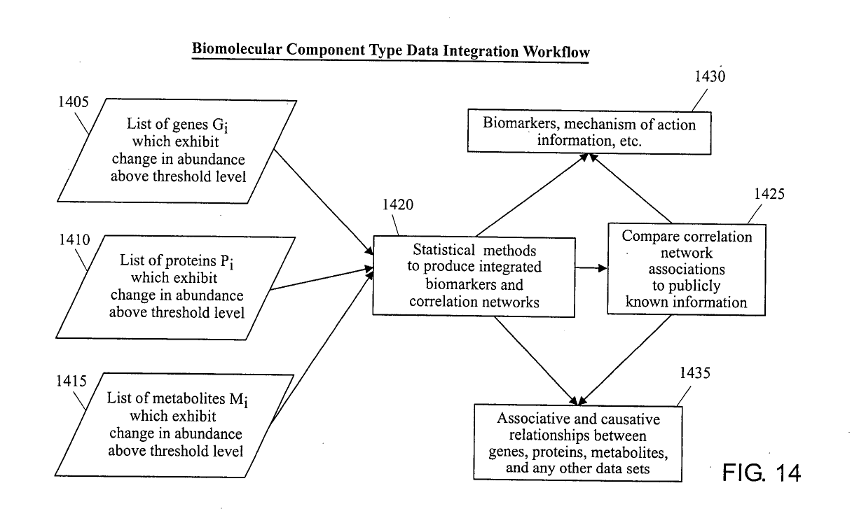

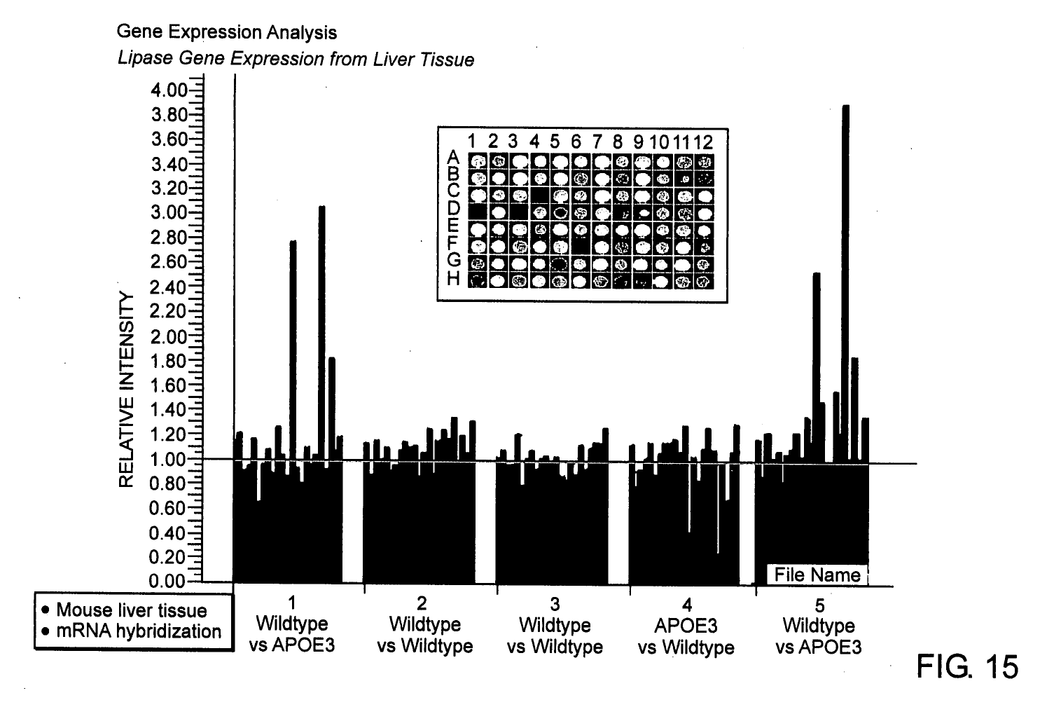

Illustrative examples of the work flow in FIG. 2. Three additional examples are disclosed herein to further illustrate the experimental methods, techniques, and analytic approaches outlined in the flow diagram illustrated in FIG. 2. More detailed flow diagrams are presented in FIGS. 11, 12, and 13, which describe preparing a data set from a biological sample and then extracting a list of either genes, proteins, or metabolites that exhibit a change in abundance above the threshold value. FIGS. 11, 12, and 13 can be understood as a higher resolution picture of FIG. 2, and in particular, focusing on Steps 205 through 220 in FIG. 2. FIG. 14 illustrates integrating the extracted list of components to produce correlation networks that can be used to compare the network associations with associations known in the literature (Steps 220, 225 and 230 in FIG. 2). To provide an even finer resolution picture of the illustrated embodiments, individual FIGS. 15-29 are presented, which map directly onto individual steps shown in FIGS. 2, 11, 12, 13 and 14.

### EXAMPLE 2

### Systems Biology Analysis of the APOE*3-Leiden Transgenic Mouse

As a test case for the application of systems biology analysis to a mammalian system, the apolipoprotein E3-Leiden (APOE*3-Leiden, APOE*3) transgenic mouse was selected. Apo E is a component of very low density lipoproteins (VLDL) and VLDL remnants and is required for receptor-mediated re-uptake of lipoproteins by the liver. [Glass and Witztum, Cell 104, 502 (1989).] The APOE*3-Leiden mutation is characterized by a tandem duplication of codons 120-126 and is associated with familial dysbetalipoproteinemia in humans. [van den Maagdenberg et al., Biochem. Biophys. Res. Commun. 165, 851 (1986); and Havekes et al., Hum. Genet. 73, 157 (1986).] Transgenic mice over expressing human APOE*3-Leiden are highly susceptible to diet-induced hyperlipoproteinemia and atherosclerosis due to diminished hepatic LDL receptor recognition, but when fed a normal chow diet they display only mild type I (macrophage foam cells) and II (fatty streaks with intracellular lipid accumulation) lesions at 9 months. [Jong et al., Arterioscler. Thromb. Vasc. Biol. 16, 934 (1996).]

APOE*3-Leiden transgenic mouse strains were generated by microinjecting a twenty-seven kilobase genomic DNA construct containing the human APOE*3-Leiden gene, the APOC1 gene, and a regulatory element termed the hepatic control region that resides between APOC1 and APOE*3 into male pronuclei of fertilized mouse eggs. The source of eggs was superovulated (C57B1/6J×CBA/J) F1 females. Transgenic founder mice were further bred with C57B1/6J mice to establish transgenic strains. Transgenic and non-transgenic littermates of F21-F22 generations were used in these experiments. All mice were fed a normal chow diet (SRM-A, Hope Farms, Woerden, The Netherlands) and sacrificed at nine weeks, at which time plasma, urine, and liver tissue samples were taken and frozen in liquid nitrogen. The samples from each individual were then subdivided for separate gene expression, protein, and metabolite analyses. The results of combined mRNA expression, soluble protein, and lipid differential profiling analyses applied to liver tissue, plasma, and urine taken from wild type and APOE*3-Leiden mice that were fed a normal chow diet and sacrificed at 9 weeks of age are presented below. Wildtype mice are used as a tool to compare the characteristics of he transgenic mice, or in other words, as control mice.

With reference to FIGS. 11-13, the biological condition 1105, 1205, 1305 to be investigated is lipid metabolism in a transgenic mammalian system, specifically atherosclerosis and hyperlipidemia in an APOE*3-Leiden transgenic mouse. The samples collected 1110, 1210, 1310 were from liver tissue, plasma, and urine taken from the transgenic mice.

Liver gene expression. Referring to FIG. 11, total mRNA was extracted from homogenized liver tissues using commercially bought, RNAeasy kits (Qiagen, Germantown, Md.). mRNA was then extracted 1115 from the total RNA preparations using a commercially bought, Oligotex kit (Qiagen, Germantown, Md.). Gene expression microarray data were acquired using the Mouse UniGene 1 spotted cDNA array (Incyte Genomics, St. Louis, Mo.). In one embodiment, an analysis of variance (ANOVA) model was selected for the design of the sample pairings that optimally reduces variation inherent in the technique.

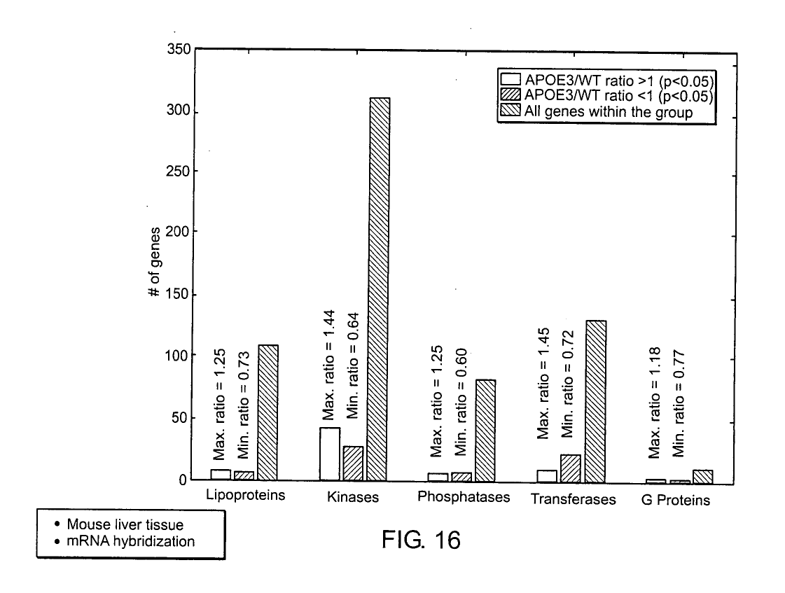

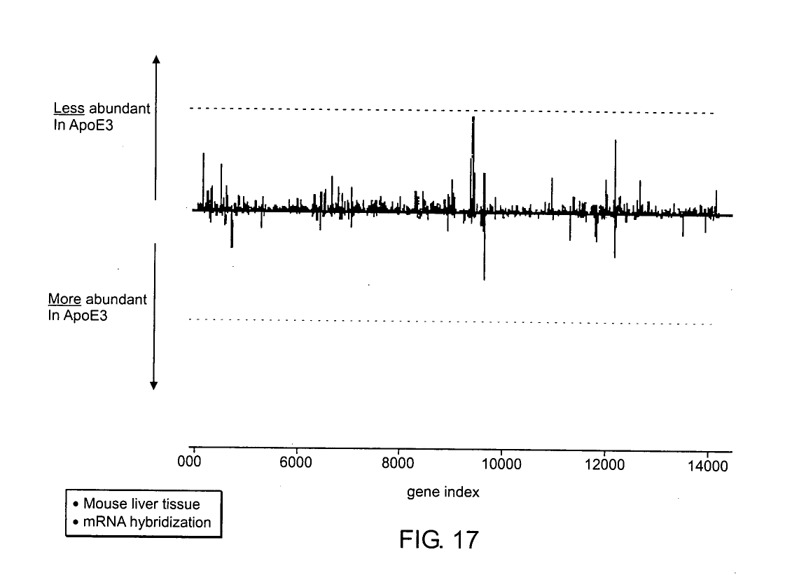

A mRNA abundance experiment 1120 was performed on the liver tissue. In one embodiment, the experiment includes mRNA hybridization. Serial analysis of gene expression and/or pattern recognition may be performed. In one embodiment, a PARC pattern recognition program is used. FIG. 15 illustrates a mRNA abundance experiment. In particular, a gene expression analysis is illustrated by a mouse liver mRNA expression ratio plot for APOE*3 transgenic mice versus wildtype mice. Examples of gene expression data sets 1125 include not only the liver gene expression analysis illustrated in FIG. 15, but also the gene expression data illustrated in FIG. 16 and the gene expression abundance results illustrated in FIG. 17.

Profiling of proteins extracted from the liver and plasma. Proteins were extracted 1215 from frozen liver tissue and plasma samples 1210. Chromatography steps 1220 may be utilized to further characterize the sample. In one embodiment, the proteins are chemically modified 1225 following the chromatography step 1220. In another embodiment, the proteins are fragmented into peptides 1230 following either the chromatography steps 1220 or the chemical modification step 1225. In one embodiment, fragmentation 1230 is performed by partial hydrolysis of the proteins. A second chromatography step 1235 may follow the fragmentation step 1230, and a mass spectrometry step 1240 may follow the chromatography step 1235. In one embodiment, a PARC pattern recognition program is used to quantify the proteins. A GIST isotopic labeling method may also be utilized. Identification of the proteins may be performed with either mass spectrometry or BioSystematics.

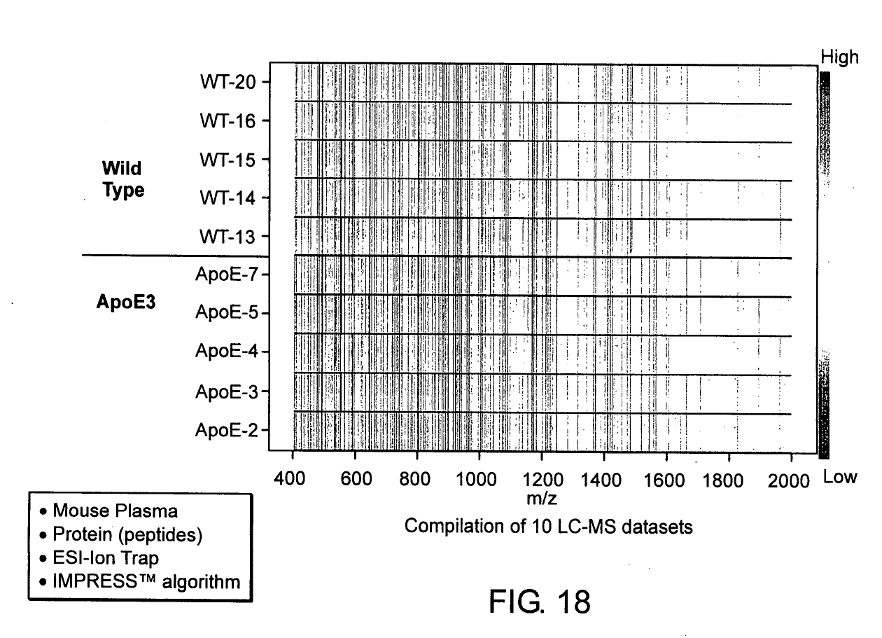

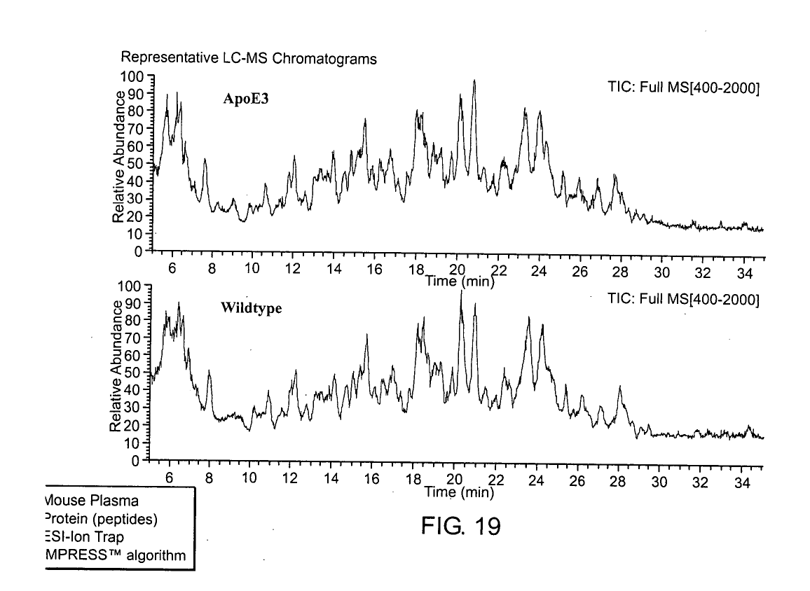

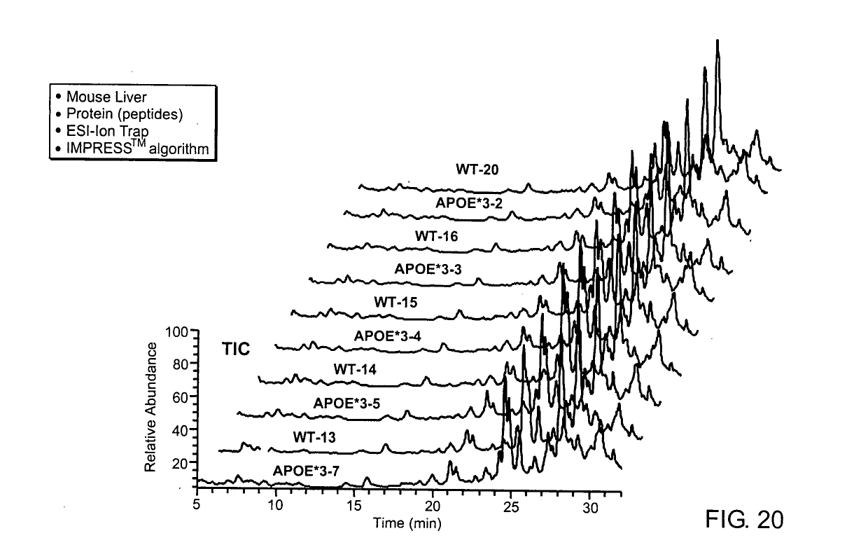

Examples of protein-derived data sets 1245 are shown in FIGS. 18-20. FIG. 18 illustrates intensity plots of LC/MS total ion chromatograms (TIC's) of plasma from APOE*3 transgenic mice vs. wildtype mice. In FIG. 19, TIC's from LC/MS profiling, which can elucidate subtle detectable differences, are shown. Both FIGS. 18 and 19 illustrate the complexity of a data set 1245, as they are included of greater than 1000 peptide peaks. FIG. 20 illustrates LC/MS chromatograms acquired from the digested liver proteins of five transgenic mice and five wildtype mice. In one embodiment, LC/MS is performed using an LCQ DecaXP (ThermoFinnigan, San Jose, Calif.) quadrupole ion trap mass spectrometer system equipped with an electrospray ionization (ESI) probe.

Profiling of metabolites extracted from urine and plasma. Metabolites were extracted from the urine and plasma samples 1310. The urine samples were profiled using one dimensional, ¹H NMR 1315. NMR spectra are one example of a data set 1340. A data set 1340 also may be generated from the plasma data by a chromatography step 1320, and then followed by a chemical modification of the metabolites 1325. The modified metabolites 1325 may be characterized by a series of chromatography 1330 and mass spectrometry 1335 steps to generate a data set 1340. In one embodiment, the plasma samples are ionized by ESI and characterized using LC/MS.

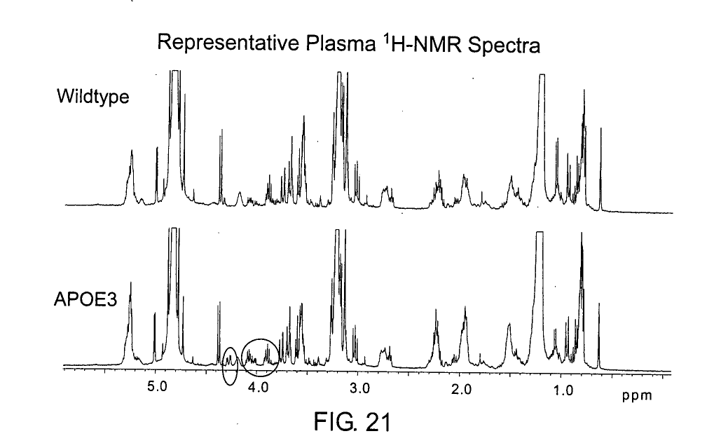

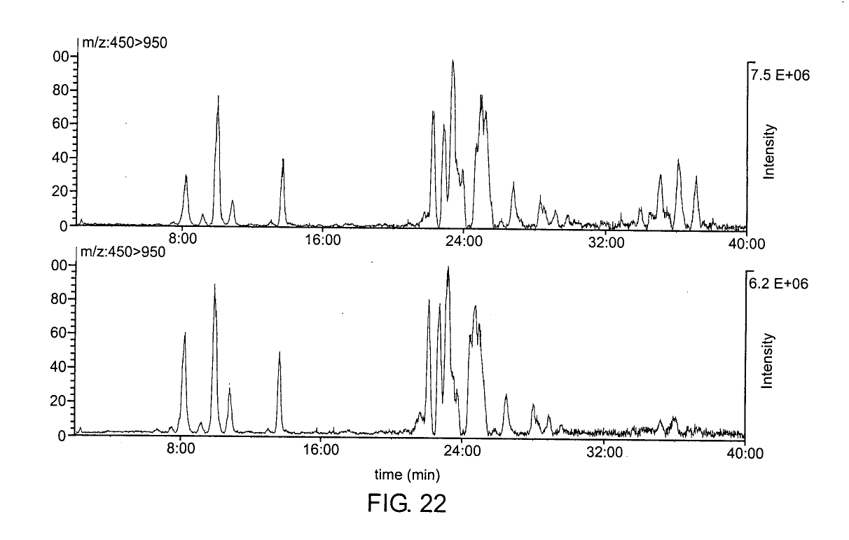

Examples of metabolite data sets 1340 are shown in FIGS. 21 and 22. FIG. 21 illustrates ¹H NMR spectra of metabolites extracted from plasma for APOE*3 and wildtype mice. After referring to the —CH₃ signal of MeOD (δ=3.30), line listings were prepared using the standard Varian NMR software. To obtain these listings, all resonances in the spectra above a threshold corresponding to about three times the signal-to-noise ratio were collected and converted to a data file format suitable for statistical analysis applications. FIG. 22 illustrates mass chromatograms of plasma lipids recorded using LC/MS for APOE*3 and wildtype mice.

Combining Data Sets. Referring back to FIGS. 11-13, in one embodiment, the gene 1125, protein 1245, and metabolite 1340 data sets are analyzed in parallel to determine molecular functions and elucidate cellular mechanisms. A number of bioinformatics tools can be utilized to link gene response, protein activity, and metabolite dynamics. The data sets 1125, 1245, 1340 are subjected to a data preprocessing step 1130, 1250, 1345 (or 210 referring to FIG. 2). An IMPRESS algorithm may be used to reduce background noise in both LC/MS chromatograms and NMR spectra. In another embodiment, the IMPRESS algorithm is used to generate IQ files for input into the PARC algorithm.

In one embodiment, data derived from the preprocessed data step 1130, 1250, 1345 is treated with a statistical analysis step 1135, 1255, 1350. Suitable forms of statistical analyses are described in more detail above. The preprocessed data may be normalized using an ANOVA algorithm. In another embodiment, normalization occurs after the statistical analysis step, which may be performed on the data sets using the PARC algorithm. In one embodiment, differentiating spectral components are identified in the factor spectra generated by the statistical analysis.

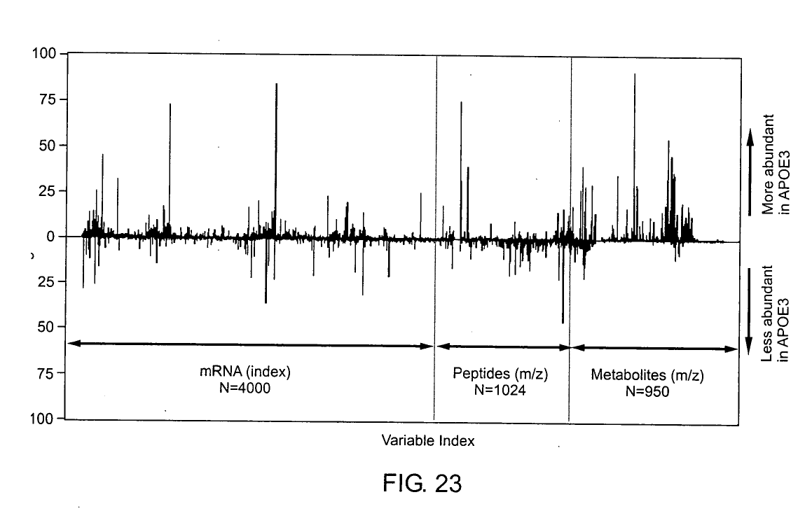

FIG. 23 depicts spectra treated by the normalization step 215. Individual gene, protein, and metabolite spectra are normalized using the model described above, and then the individual normalized spectra are concatenated into a single factor spectrum. In FIG. 23, the data measured on a biological sample extracted from mouse liver. Using the concatenated spectrum, direct comparison across biomolecular component types may be performed.

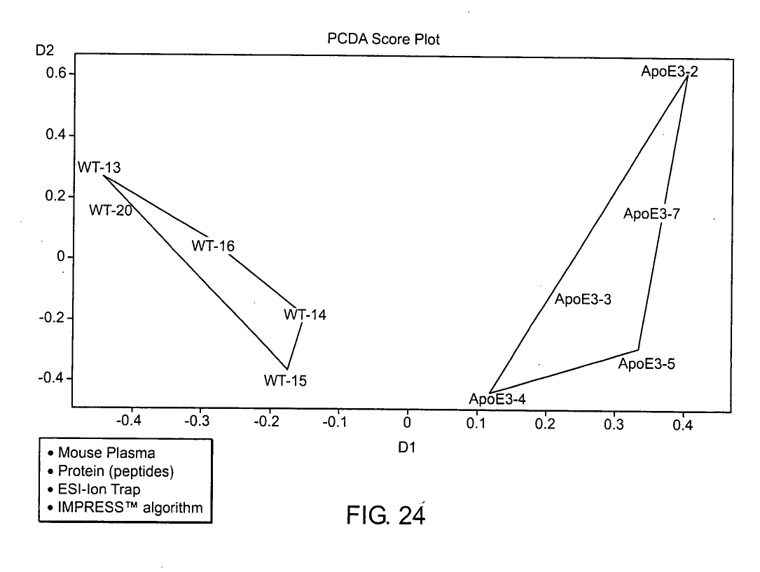

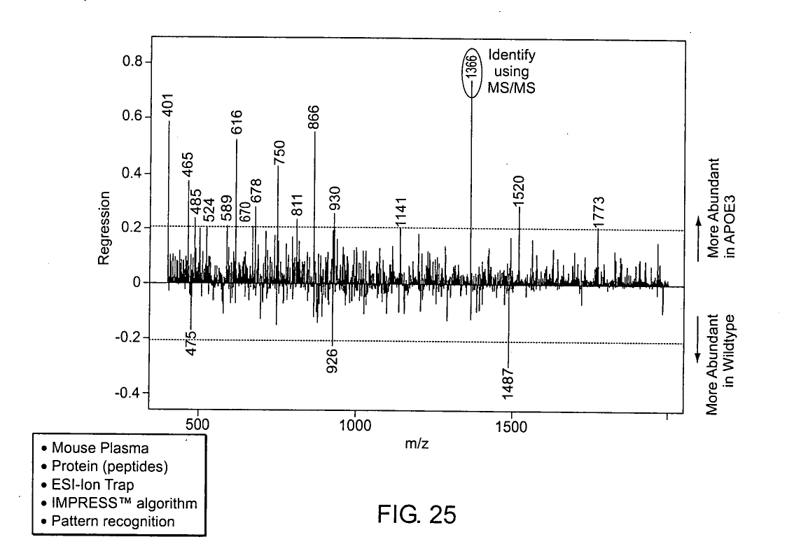

FIGS. 24-25 provide an illustrative embodiment of the statistical analysis step 1135, 1255, 1350 and the subsequent inspection step 1140, 1260, 1355. For the sake of simplicity, only the protein plasma analysis is presented, but the method can be extended to both genes and metabolites. FIG. 24 illustrates clustering of wildtype mouse data and APOE*3 transgenic mouse data performed using a PC-DA 1255 on the peptide ion mass data. An inspection 1260 of the two distinct clusters shown in FIG. 24 reveals that the masses of the ions differentiate the two clusters. FIG. 25 shows the masses of the peptide ions exhibiting significant differences plotted in a difference factor spectrum. In one embodiment, a t-test is applied to each of the differentiating ions to test their significance. In another embodiment, loading plots are used instead of factor spectra.

An additional mass spectroscopy analysis step 1265, 1360 may be performed to analyze further the proteins, peptides, or metabolites that exhibit a change above a threshold abundance level. In one embodiment, MS/MS is used to analyze and identify the proteins, peptides, or metabolites. In another embodiment, genes, proteins, peptides, or metabolites that exhibit a statistically significant change are identified during the manual inspection step 1140, 1260, 1335. Subsequent to identifying all genes, proteins, peptides, and metabolites 1145, 1270, 1365, a list of those genes, proteins, peptides, and metabolites is extracted and stored 1150, 1275, 1370 for future comparison.

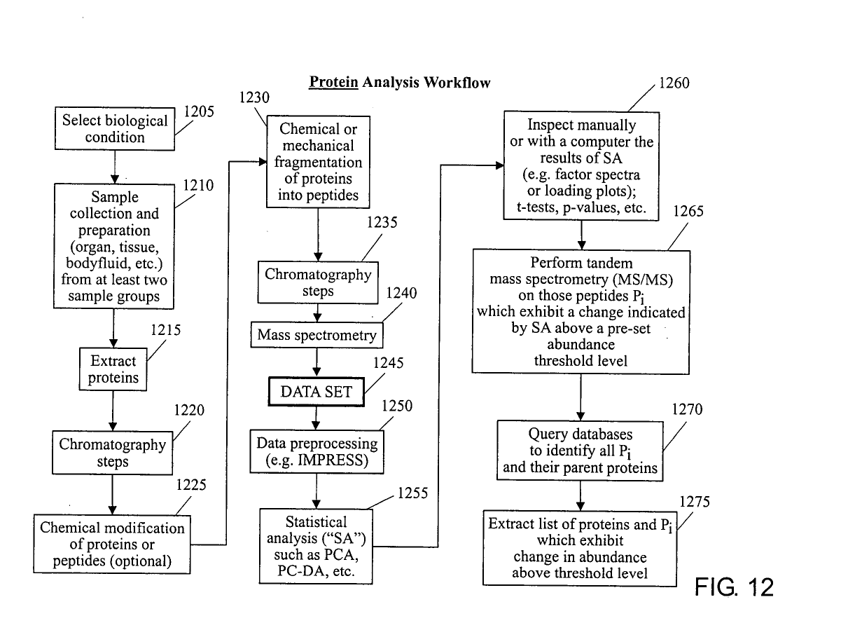

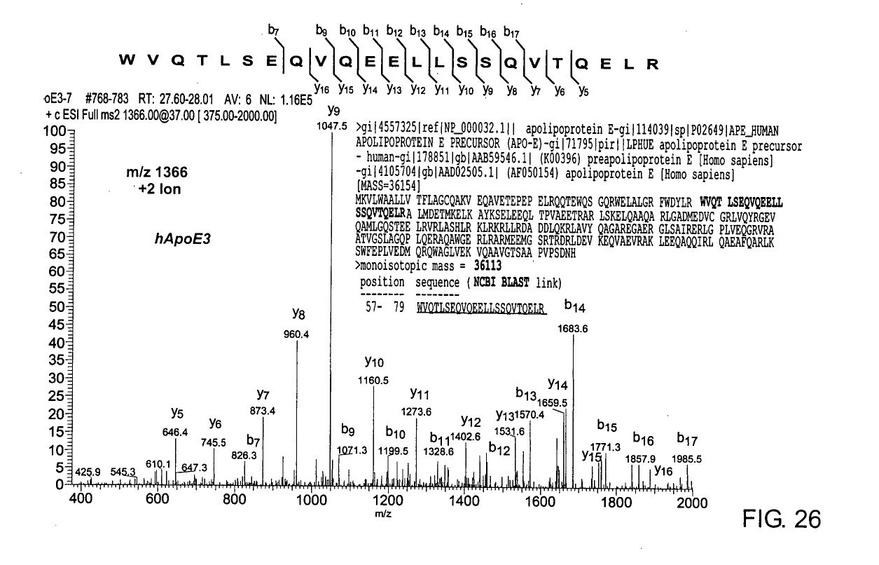

FIG. 26 depicts an MS/MS spectrum of the peptides generated by hydrolysis of the proteins extracted from mouse plasma, which corresponds to step 1265 in FIG. 12. Those peptide fragments, which are labeled b7-b17 and y5-y16, are compared to a database, so that the protein which was fragmented can be identified and sequenced, which corresponds to the identification step 1270 in FIG. 12. In this particular case the protein identified is human ApoE3 which is the protein introduced by the transgenic manipulation.

Table I lists the key differentially expressed components extracted from the lists of genes, proteins, and metabolites. This list was generated in accord with steps 1150, 1275, 1370, which are illustrated in FIGS. 11-13. The extracted list of components also corresponds to the extract list of components step 220 in FIG. 2.

TABLE I
Key differentially expressed biomolecular components (Excluding human ApoE3).

| Biomolecular | | | Fold Ratio |
|---|---|---|---|
| component type | Component ID | Name | (APOE3:WT) |
| Gene | G 7801 | Heat shock 70 KD protein | 3.10 |
| Gene | G 562 | RIKEN cDNA 3230402M22 | 2.72 |
| Metabolite | M 1 | Trigycerides | 2.59 |
| Metabolite | M 7 | DAG C18, 20:1 | 1.92 |
| Metabolite | M 9 | LysoPC C16:0 | 1.68 |
| Gene | G 7485 | Apoptosis inhibitory 6 | 1.51 |
| Protein | P 1059 | FABP (fatty acid binding protein) | 1.36 |
| Gene | G 1615 | Heterogeneous nuclear RNP H1 | 1.35 |
| Gene | G 693 | FABP (fatty acid binding mRNA) | 1.33 |
| Gene | G 1032 | Translation Initiation Factor 2 | 1.14 |
| Metabolite | M 3 | PC C20, 20:8 | 0.94 |
| Gene | G 8147 | Apolipoprotein A1 | 0.76 |
| Protein | P 744 | Protein Kinase C, epsilon | 0.74 |
| Protein | P 451 | ATP-binding cassette (ALD), mem1 | 0.72 |
| Protein | P 1439 | Heme oxygenase-2 | 0.64 |
| Protein | P 1362 | IPF1 | 0.59 |

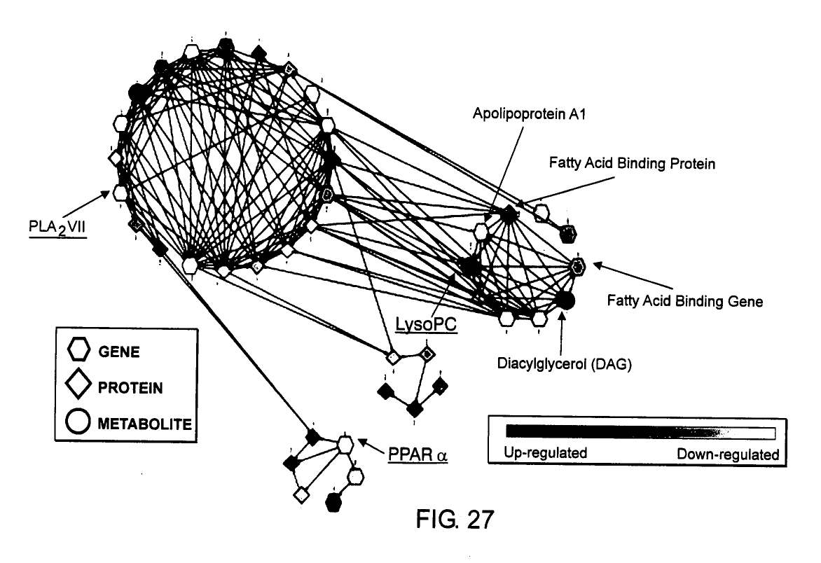

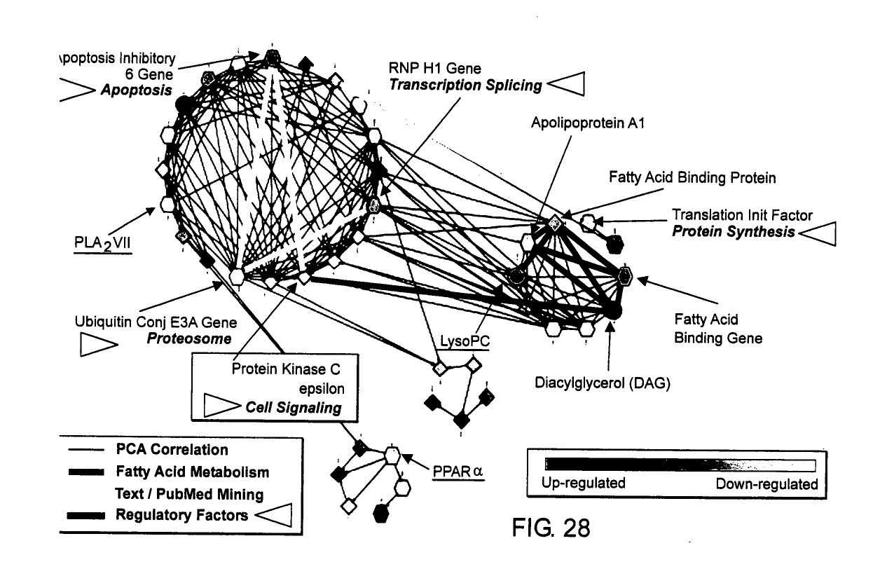

In one embodiment, the individual biomolecular components listed in Table I are normalized, so a more meaningful comparison across biomolecular component types can be performed. In another embodiment, the list of biomolecular components listed in Table I are used to produce a correlation network in accord with step 225 in FIG. 2 and step 1420 in FIG. 14. FIG. 27 illustrates a correlation network between biomolecular component types. The network was produced with a non-linear PCA feature correlation and illustrates potential associations between individual biomolecular components. The correlation network associations then may be compared to existing knowledge from the literature or other public information sources, which corresponds to step 230 in FIG. 2 or step 1425 in FIG. 14. FIG. 28 illustrates a map of the known relations between the correlation network association and published information.

Referring back to FIG. 14, an illustrative embodiment of correlation network associations that are analyzed to determine biomarkers or mechanisms of action 1430 is depicted. The known relations may be analyzed to determine biomarkers or mechanisms of action 1430. In one embodiment, the correlation network associations are used to determine associative and causative relationships across biomolecular component types 1435. The known relations also may be used to determine associative and causative relationships across biomolecular component types 1435.

Returning to FIG. 2, in one embodiment, the system is perturbed 235. As stated above, the perturbed system then may be used to produce new data sets, new correlations networks, and new correlation network associations before deducing the causal mechanisms of the perturbation. The perturbations to the system may be iterated until causal relations are determined between multiple bimolecular component types.

From the biomarkers determined from a systems biology analysis, similar to the one described above, markers that differentiate diseased and healthy populations may be derived. This information can then be placed in the appropriate biological context to determine, e.g., when a marker can be identified as either a causative agent or a downstream product of a disregulated pathway. As described above, comprehensive gene, protein, and metabolite profiling, coupled with correlation analysis and network modeling, provide insight into biological context, and this level of knowledge may be used to develop therapeutic agents or may serve as a basis for directed, hypothesis-driven experiments that are designed to further elucidate pathophysiologic mechanisms.

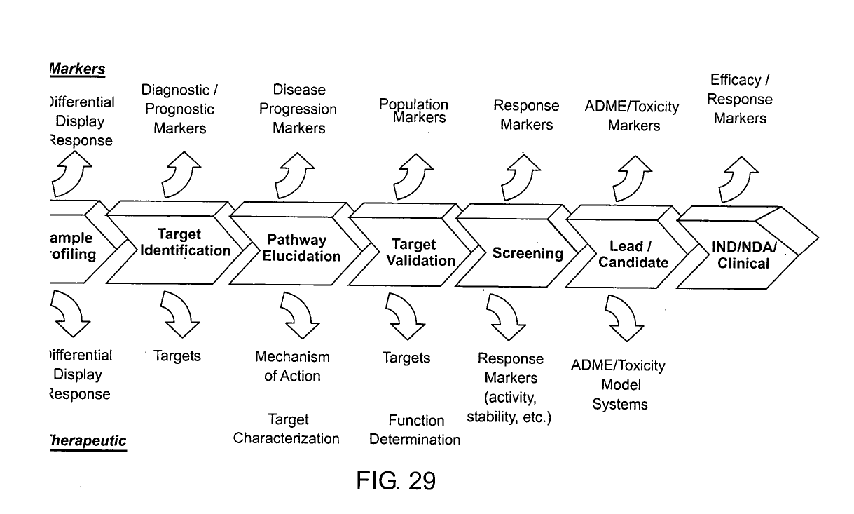

FIG. 29 illustrates typical “offerings” or “deliverables,” in terms of biomarkers or therapeutic agents that can be derived from a systems biology analysis. Described below are two examples that illustrate not only typical systems biology analyses, but also a more detailed description of how the information derived from these systems biology analyses is employed to determine not only which therapeutic agents should be used, but also which pathophysiologic mechanisms require further study.

### EXAMPLE 3

### Systems Biology Analysis of the APOE*3-Leiden Transgenic Mouse

The results of combined mRNA expression, soluble protein, and lipid differential profiling analyses applied to liver tissue, plasma, and urine taken from wild type and APOE*3-Leiden mice that were fed a normal chow diet and sacrificed at 9 weeks of age are presented below. Results from each biomolecular component type class analysis reveal the presence of early markers of predisposition to disease. In addition, results of a correlation analysis are suggestive of networks of molecules—spanning genes, proteins and lipids—that undergo concerted change.

Animals. APOE*3-Leiden transgenic mouse strains were generated by microinjecting a twenty-seven kilobase genomic DNA construct containing the human APOE*3-Leiden gene, the APOC1 gene, and a regulatory element termed the hepatic control region that resides between APOC1 and APOE*3 into male pronuclei of fertilized mouse eggs. The source of eggs was superovulated (C57B1/6J×CBA/J) F1 females. Transgenic founder mice were further bred with C57B1/6J mice to establish transgenic strains. Transgenic and non-transgenic littermates of F21-F22 generations were used in these experiments. All mice were fed a normal chow diet (SRM-A, Hope Farms, Woerden, The Netherlands) and sacrificed at nine weeks, at which time plasma, urine, and liver tissue samples were taken and frozen in liquid nitrogen. The samples from each individual were then subdivided for separate gene expression, protein, and metabolite analyses.

Liver gene expression. Total mRNA was extracted from homogenized liver tissues using commercially bought, RNAeasy kits (Qiagen, Germantown, Md.). mRNA was then extracted from the total RNA preparations using a commercially bought, Oligotex kit (Qiagen, Germantown, Md.). Gene expression microarray data were acquired using the Mouse UniGene 1 spotted cDNA array (IncyteGenomics, St. Louis, Mo.). An analysis of variance (ANOVA) model was selected for the design of the sample pairings that optimally reduces variation inherent in the technique.

Liver protein profiling. Frozen liver tissues were powdered in a pre-chilled mortar that was kept cold with the addition of liquid nitrogen. T-PER protein extraction reagent (Pierce Chemical Co., Rockford, Ill.) was then added at 8 μL/mg of tissue, and the sample was further homogenized by sonication. Samples were then centrifuged at 10,000×g for 5 minutes, and the supernatants collected. Relative total protein concentrations were determined from integrated whole-chromatograms of aliquots that had been injected into a size exclusion chromatography system, consisting of a Super SW3000 TSKgel column (Tosoh Biosep, Tokyo) and an LC Packings Ultimate pump (Dionex, Marlton, N.J.). To reduce sample complexity, the protein supernatants were fractionated via reversed-phase chromatography on a VISION Workstation (Applied Biosystems, Foster City, Calif.) equipped with a POROS R2/H column (4.6×100 mm) (Applied Biosystems, Foster City, Calif.) that was eluted with a water/acetonitrile (MeCN) gradient in the presence of 0.1% trifluoroacetic acid (TFA). Proteins were digested, thermally denatured and reduced in 100 mM ammonium bicarbonate, 5 mM calcium chloride and 10 mM dithiothreitol at 75° C. for 30 minutes, alkylated with 25 mM iodoacetamide at 75° C. for 30 minutes, and then digested with 0.3% (w/w trypsin/protein) for 24 hours at 37° C.

Protein LC/MS analyses. Liquid chromatography-tandem mass spectrometry (LC/MS) was performed using an LCQ DecaXP (ThermoFinnigan, San Jose, Calif.) quadrupole ion trap mass spectrometer system equipped with an electrospray ionization probe. The LC component consisted of a Surveyor autosampler and quaternary gradient pump (ThermoFinnigan, San Jose, Calif.). Samples were suspended in mobile phase and eluted through a Vydac low-TFA C 18 column (150×1 mm, 5 μm) (GraceVydac, Hesperia, Calif.). The column was eluted at 50 μL/minute isocraticly for two minutes with Solvent A (water/MeCN/acetic acid/TFA, 95/4.95/0.04/0.01, vol/vol/vol/vol) followed by a linear gradient over 43 minutes to 75% Solvent B (water/MeCN/acetic acid/TFA, 20/79.95/0.04/0.01, vol/vol/vol/vol). The electrospray ionization voltage was set to 4.25 kV and the heated transfer capillary to 200° C. Nitrogen sheath and auxiliary gas settings were 25 and 3 units, respectively. For quantification of tryptic peptides, the scan cycle consisted of a single full scan mass spectrum acquired over m/z 400-2000 in the positive ion mode. Data-dependent product ion mass spectra (MS/MS) were also acquired for peptide identification using the TurboSEQUEST algorithm (ThermoFinnigan, San Jose, Calif.).

Liver lipid profiling. Liver tissue was freeze-dried, pulverized, and then extracted with 20 μL isopropanol per mg of tissue in an ultrasonic bath for 2 hours. The samples were then centrifuged and the supernatants collected. Samples were then diluted with 4 volumes of water and taken for LC/MS analysis. LC/MS data were acquired using an LCQ (ThermoFinnigan, San Jose, Calif.) quadrupole ion trap mass spectrometer equipped with an electrospray ionization probe. The LC component consisted of a Waters 717 series autosampler and a 600 series single gradient forming pump (Waters, Milford, Mass.). Samples were injected in duplicate, in random order, onto an Inertsil column (ODS 3.5 mm, 100×3 mm) protected by an R2 guard column (Chrompack). Three mobile phases were used in the elution: (1) (water/MeCN/ammonium acetate/formic acid, 93.9/5/1/0.1, vol/vol/vol/vol), (2) (acetonitrile/isopropanol/ammonium acetate/formic acid, 68.9/30/1/0.1, vol/vol/vol/vol), and (3) (isopropanol/dichloromethane/ammonium acetate/formic acid, 48.9/50/1/0.1, vol/vol/vol/vol). The column was eluted at 0.7 mL/minute using a two-step gradient: Step (1) from 0 to 15 minutes beginning with 70% A, 30% B, 0% C and ending with 5% A, 95% B and 0%, and Step (2) a 20 minute gradient with no change in A, 95% to 35% B, and 0% to 60% C. The electrospray ionization voltage was set to 4.0 kV and the heated transfer capillary to 250° C. Nitrogen sheath and auxiliary gas settings were 70 and 15 units, respectively. For quantification of metabolites, the scan cycle consisted of a single full scan (1 s/scan) mass spectrum acquired over m/z 250-1200 in the positive ion mode.

LC/MS data pre-processing. LC/MS data sets were converted into ANDI (.cdf) format using the File Converter functionality built into the Xcaliber instrument control software (ThermoFinnigan, San Jose, Calif.). The IMPRESS algorithm (TNO Pharma, Zeist, The Netherlands) was then applied to the converted files for automated peak detection and peak data quality assessment. The program evaluates each mass trace for its chromatographic quality by assessing its information content. The LC/MS chromatogram at each mass to charge ratio were smoothed to remove noise spikes and then the entropy of the trace was calculated using Equation 12. Taking the reciprocal value of H and scaling all results to the largest value gave each mass trace a scaled chromatographic quality number called the Impress Quality (IQ):

$$H = - {\sum }_{i = 1}^{N} {p}_{i}^{2} log ( {p}_{i}^{2} ) . \qquad ( 12 )$$

An IQ threshold was then selected, and if the IQ of a peak was below the threshold, the peak was deemed to be of poor quality and was not taken forward to clustering analyses described below.

Normalization of microarray data. As described above, the data may be represented by the following model:
y_{gik}=μ_{gv}+Aᵢ+Dₖ+ε_{gik}, (13)
where the gene and variety effects are described by μ_{gv}, the array effect by Aᵢ, the dye effect by Dₖ, and the error by ε_{gik}. The error is normally distributed with zero mean, and the variance, σ_{gv}², is not permitted to be different for each gene and variety. The optimal parameters of the model are calculated using a maximum likelihood estimator. For each particular array and dye, the samples are then scaled as:
\tilde{y}_{gik}=y_{gik}−Aᵢ−Dₖ. (14)

Statistical tests of significance. To estimate the statistical significance of difference mean normalized intensities from transgenic and wild type samples, a t-test was applied for each of the N genes, and the corresponding p-values were calculated. When assessing the statistical significance of fold change for each gene, a total N p-values were collected, so several p-values with p≦0.05 were expected. To account for this, the overall likelihood P(p), of observing a p-value ≦p for any of the N genes was used. Assuming independence of all genes, the overall likelihood was estimated with:
P(p)≈1−(1−p)^{N}. (15)

PCDA analysis and correlation plots. Principal component and discriminant analyses (PCDA) were applied to the tryptic peptide and lipid LC/MS profiles that had been pre-processed with the IMPRESS algorithm as described above. This was done using WINLIN statistical software (TNO Pharma, Zeist, The Netherlands).

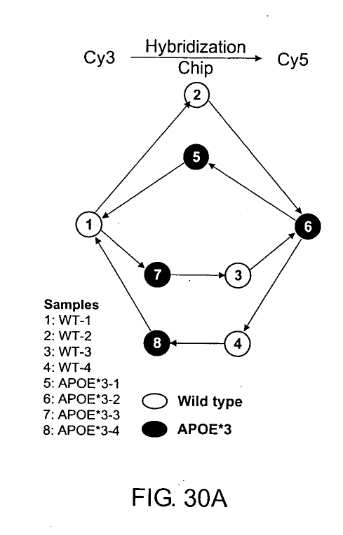

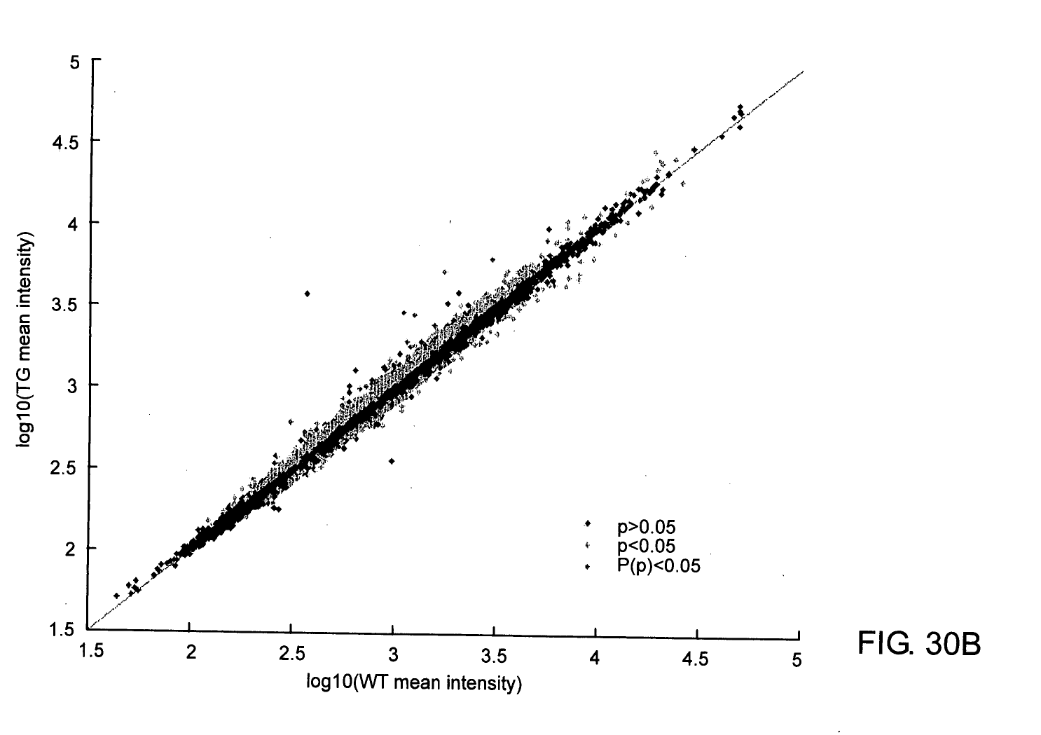

Microarray analysis of liver gene expression. Mouse liver mRNA samples were paired for hybridization on the UniGene 1 cDNA spotted microarrays following the “loop design” shown in FIG. 30A. This method of pairing was based on an ANOVA model that was designed to provide a basis for optimal normalization of gene expression data and to minimize the contribution of variability that might arise from factors, such as unequal rates of hybridization between nucleic acids or dye effects. mRNA samples were labeled with Cy3 and Cy5 for dual hybridization, as shown.

As evidenced by the cDNA microarray data scatter plot shown in FIG. 30B, relatively few genes were differentially expressed at the 95% confidence level. Values were plotted as mean values of expression in wild type and APOE*3-Leiden transgenic mice, and data points were color-coded on the basis of statistical significance. Far fewer met a more rigorous overall likelihood, P(p), assessment that attempts to rule out chance events where data may randomly, but falsely, have p-values <0.05.

Table II lists a sample set of genes where the fold-ratio between transgenic and wild type control was either less than 0.8 or greater than 1.2. The relatively low p-values that were observed despite the rather narrow margins of difference in expression reflect the statistical advantages of the ANOVA model. Of note are the lower levels of expression of apolipoprotein AI and an analog of apolipoprotein B in the transgenic animals, while an analog of apolipoprotein F was higher. Interestingly, prior analysis of plasma obtained from the APOE*3-Leiden mice revealed an approximately two-fold down regulation at the protein level. In addition, peroxisomal proliferator-activated receptor-alpha (PPARα) expression was not different between the two populations, while liver fatty acid binding protein (L-FABP) was 43% higher in the transgenics. PPARα plays a key role in initiating gene expression of proteins involved in lipid metabolism, while experimental evidence suggests that L-FABP may control the activity of the transcription factor by controlling the rate of presentation of activating ligand. The lipid profiling analysis shows that lipid metabolism is indeed impacted by the presence of the transgene, and in the absence of change in PPARα levels, these data support a regulatory role for L-FABP.

TABLE II
Liver mRNA expression.

| Description | Ratio | p-value |
|---|---|---|
| claudin 4 | 0.59 | 0.001 |
| CD8beta opposite strand | 0.69 | 0.003 |
| iroquois related homeobox 3 (Drosophila) | 0.72 | 0.001 |
| cysteine rich protein | 0.74 | 0.006 |
| Apolipoprotein A-I | 0.75 | 0.009 |
| fatty acid binding protein 5, epidermal | 0.75 | 0.044 |
| ESTs, Moderately similar to I56333 apolipoprotein | 0.75 | 0.043 |
| B - rat | | |
| plexin 6 | 0.77 | 0.019 |
| nitric oxide synthase 3, endothelial cell | 0.81 | 0.018 |
| ornithine aminotransferase | 1.22 | 0.016 |
| glutathione S-transferase, alpha 1 (Ya) | 1.28 | 0.029 |
| malate dehydrogenase, mitochondrial | 1.28 | 0.002 |
| extracellular proteinase inhibitor | 1.28 | 0.027 |
| CD53 antigen | 1.28 | 0.037 |
| ESTs, Weakly similar to apolipoprotein F [H. sapiens] | 1.28 | 0.028 |
| receptor (calcitonin) activity modifying protein 3 | 1.29 | 0.032 |
| cytochrome c oxidase, subunit VIIc | 1.29 | 0.040 |
| eosinophil-associated ribonuclease 2 | 1.31 | 0.013 |
| cytochrome c oxidase, subunit VIIa 3 | 1.32 | 0.044 |
| histidine triad nucleotide-binding protein | 1.33 | 0.031 |
| malate dehydrogenase, soluble | 1.33 | 0.023 |
| M. musculus H2B gene | 1.34 | 0.021 |
| ATPase, H+ transporting lysosomal (vacuolar proton | 1.34 | 0.048 |
| pump) | | |
| ATP synthase, H+ transporting, mitochondrial F0 | 1.39 | 0.018 |
| complex | | |
| thymosin, beta 4, X chromosome | 1.40 | 0.024 |
| ganglioside-induced differentiation-associated-protein 3 | 1.40 | 0.012 |
| solute carrier family 35 (UDP-galactose transporter), | 1.42 | 0.024 |
| member 2 | | |
| glucose regulated protein, 58 kDa | 1.43 | 0.021 |
| spermidine/spermine N1-acetyl transferase | 1.43 | 0.030 |
| fatty acid binding protein 1, liver | 1.43 | 0.024 |
| signal recognition particle 9 kDa | 1.45 | 0.034 |
| orosomucoid 2 | 1.46 | 0.020 |
| cathepsin S | 1.48 | 0.033 |
| Lysozyme | 1.49 | 0.007 |
| nucleobindin 2 | 1.50 | 0.015 |
| orosomucoid 1 | 1.51 | 0.009 |
| serum amyloid A 3 | 1.51 | 0.001 |
| major urinary protein 1 | 1.56 | 0.005 |
| DnaJ (Hsp40) homolog, subfamily C, member 3 | 1.58 | 0.012 |
| SEC61, gamma subunit (S. cerevisiae) | 1.60 | 0.008 |
| calcium binding protein A11 (calgizzarin) | 1.70 | 0.004 |
| tumor rejection antigen gp96 | 2.01 | 0.003 |
| proteoglycan, secretory granule | 2.45 | 0.001 |
| heat shock 70 kD protein 5 (glucose-regulated | 2.93 | 0.001 |
| protein, 78 kD) | | |

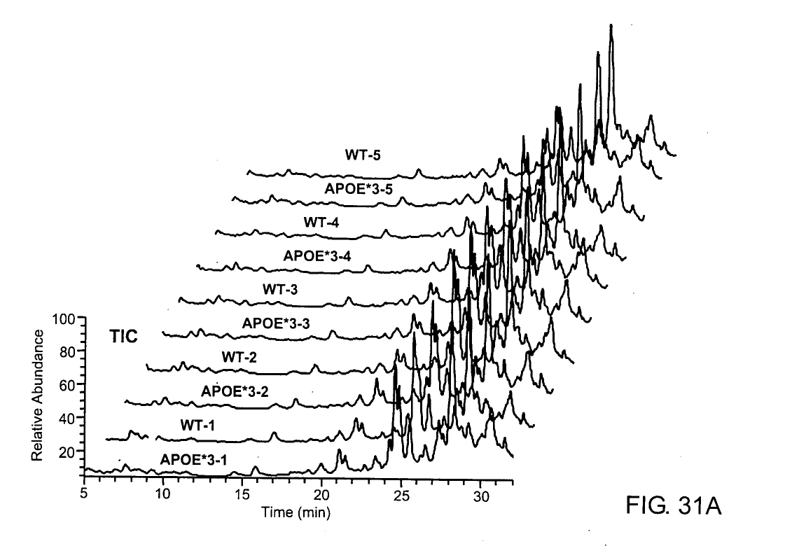

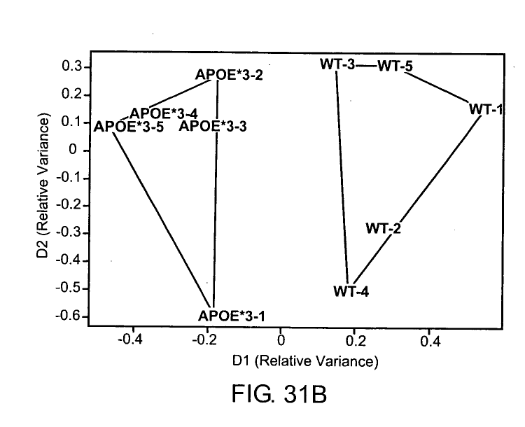

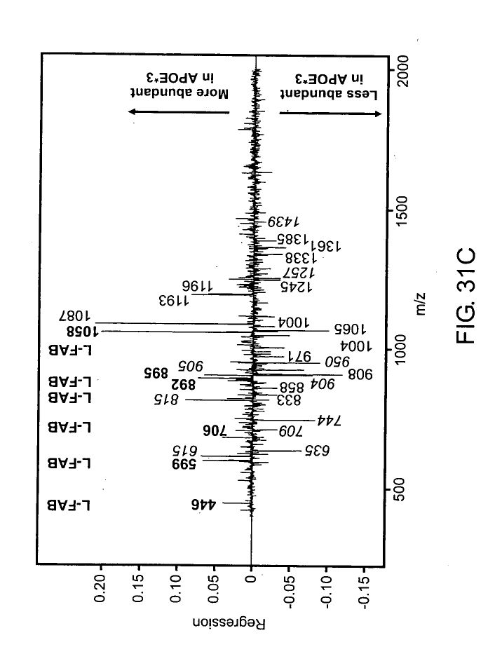

Quantitative profiling of liver proteins. Off-line reversed phase separation of soluble liver proteins to decrease the sample complexity by approximately a factor of 20 was initially employed. An ESI-LC configuration was coupled to the mass spectrometer that was capable of handling hundreds of consecutive injections. Next, data was acquired using an MS-only scan cycle, without acquisition of sequencing MS/MS scans, To reduce cycle time and minimize the loss of information that occurs while the column elutes between scans. As shown in FIG. 31A, LC/MS chromatograms were acquired for digested liver protein fractions from five APOE*3-Leiden and five wild type mice. The IMPRESS algorithm was then applied to each data set to extract peak intensity and signal quality information. An IMPRESS quality value of 0.5 was selected as the threshold below which poor quality signal data would be excluded from further analysis. Clustering was then performed using the principal component-discriminant analysis (PCDA) tool built into the WINLIN software. As shown in FIG. 31B, two distinct clusters were observed with transgenic mice in one and wild type mice in the other. An inspection of the factor spectrum, illustrated in FIG. 31C, provided masses of the ions that differentiated the two clusters. A t-test was applied to each of the differentiating ions to test significance, and an LC/MS/MS spectrum was acquired for each peptide. Six tryptic peptides that were each derived from a digestion of L-FABP, with mass to charge ratios 446, 599, 706, 892, 895, and 1058, are labeled in FIG. 31C. Since the factor spectrum is semi-quantitative in nature, peak intensity information gathered by IMPRESS was used to calculate relative differences. The results of this profiling analysis indicated that L-FABP was up-regulated by 44% in transgenic mice relative to wild type controls. This was essentially a one-to-one correlation with the mRNA expression observation noted above. Table III summarizes the results of the protein analysis.

TABLE III
Liver protein expression.

| | Ratio | p- |
|---|---|---|
| Description | TG/WT | value |
| Apoptosis Protein MA-3 | 0.85 | 0.019 |
| Asialoglycoprotein Receptor 1 | 1.39 | 0.028 |
| ATP-Binding Cassette, Sub-Family D (ALD), Member 1 | 0.72 | 0.025 |
| Beta-Crystallin B2 | 0.76 | 0.016 |
| Efa3_Mouse Ephrin-A3 (Eph-Related Receptor Tyrosine | 0.52 | 0.005 |
| Kinase Ligand 3) | | |
| Liver-Fatty Acid Binding Protein | 1.44 | 0.036 |
| Forkhead Transcription Factor Fkh-1 | 1.24 | 0.008 |
| Glutathione-S-Transferase | 0.69 | 0.015 |
| Guanine Nucleotide Exchange Factor Eif-2B Delta | 1.36 | 0.002 |
| Chain, Long Form | | |
| Guanine Nucleotide-Exchange Activator CDC25 | 1.59 | 0.014 |
| Homolog | | |
| Heme Oxygenase-2; HO-2 | 0.64 | 0.014 |
| Hemopoietic Cell Phosphatase, Tyrosine Phosphatase | 1.38 | 0.020 |
| Homeotic Protein Mh19 Precursor | 0.48 | 0.060 |
| Lithium-Sensitive Myo-Inositol Monophosphatase A1 | 0.62 | 0.034 |
| Killer Cell Lectin-Like Receptor, Subfamily A, | 1.98 | 0.019 |
| Member 6 | | |
| Lymphocyte Antigen 78 | 0.53 | 0.007 |
| Md6 Protein | 0.67 | 0.035 |
| Mouse Fat 1 Cadherin | 0.85 | 0.019 |
| Noda_Mouse Nodal Precursor | 0.52 | 0.006 |
| Numb-Binding Protein Lnxp80 | 1.12 | 0.032 |
| Probable E1-E2 Atpase - Mouse (Fragment) | 0.83 | 0.034 |
| Procollagen, Type V, Alpha 2 | 1.20 | 0.012 |
| Protein Kinase C, Epsilon | 0.74 | 0.105 |
| Pyruvate Kinase | 1.24 | 0.008 |
| Ubiquitin-Protein Ligase E3a | 1.15 | 0.079 |

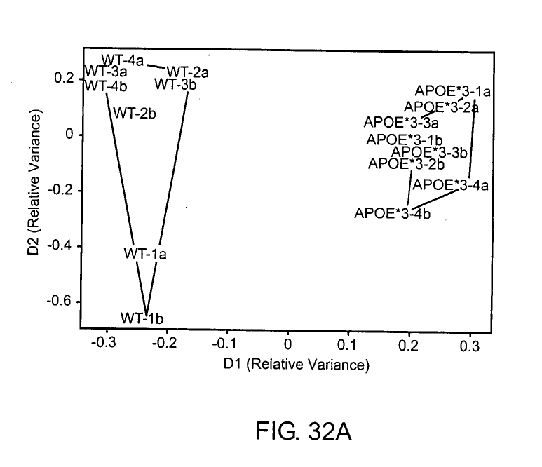

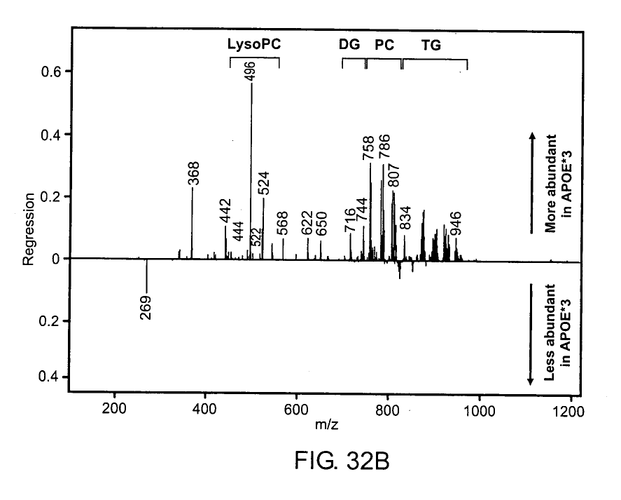

Quantitative profiling of liver lipids. Lipids were profiled using a strategy similar to that used for the protein analysis. Duplicate datasets were acquired for each animal. The extraction protocol and LC system was designed to fractionate larger, non-polar lipids such as diacylglycerols (DG) and triacylglycerols (TG). Captured within this acquisition were also quantitative profiles of phosphatidylcholine (PC) and lysophosphatydylcholine (LysoPC) lipids. Following data pre-processing with IMPRESS to obtain peak information, PCDA clustering analysis was performed using WINLIN. As shown in FIG. 32A, the two populations of mice formed two distinct clusters. The PCDA factor spectrum, illustrated in FIG. 32B, indicates that a number of lipids contribute to the difference between to the two populations. Mass to charge ratio ranges that include the majority of lysophosphatidylcholines (LysoPC), diacylglycerols (DG), phosphatidylcholines (PC), and triacylglycerols (TG) are indicated.

As summarized in Table IV, a number of triacylglycerols were higher in the transgenic mice, while none were found to be in lower abundance. Similarly, two lysophosphatidylcholines, 1-palmitoyl-2-hydroxy-sn-glycero-3-phosphocholine (LysoPC C16:0) and 1-Stearoyl-2-Hydroxy-sn-Glycero-3-Phosphocholine (LysoPC C18:0), were found at higher levels in the APOE*3-Leiden mice, while there were no significant differences observed for other LysoPCs. Interestingly, among the diacylglycerol and phosphatidlycholine sub-classes, an overall trend toward higher abundance in the transgenic animals was not observed, suggesting that the disruption of lipid metabolism imposed by insertion of the transgene leads to a complex, multifactoral change in the regulation of lipid levels.

TABLE IV
Liver lipid: Fold difference between APOE*3-Leiden transgenic mice
and the wildtype control mice.

| | | Ratio | |
|---|---|---|---|
| Description | Species | TG/WT | p-value |
| Lysophosphatidylcholine | C16:0 | 1.31 | 0.0190 |
| | C18:0 | 1.24 | 0.0241 |
| Diacylglycerol | C18, C20:1 | 1.43 | 0.0064 |
| | C22, 20:1 | 0.78 | 0.0151 |
| | C22, 22:10 | 0.80 | 0.0018 |
| | C22, 22:3 | 0.77 | 0.0070 |
| Phosphatidylcholine | C18, 18:0 | 0.75 | 0.0092 |
| | C20, 18:2 | 0.79 | 0.0231 |
| | C20, 20:8 | 0.77 | 0.0422 |
| | C20, 20:7 | 0.82 | 0.0341 |
| | C20, 20:4 | 1.50 | 0.0138 |
| | C20, 22:3 | 2.75 | 0.0001 |
| | C20, 22:2 | 1.85 | 0.0023 |
| | C20, 22:1 | 1.20 | 0.0059 |
| | C22, 22:4 | 2.82 | 0.0005 |
| | C22, 22:3 | 1.84 | 0.0002 |
| | C22, 22:2 | 1.37 | 9.4E−06 |
| Triacylglycerol | C50:0 | 2.02 | 9.7E−07 |
| | C56:7 | 1.87 | 6.9E−06 |
| | C56:6 | 1.96 | 2.8E−08 |
| | C56:5 | 1.60 | 0.0003 |
| | C56:4 | 1.97 | 0.0000 |
| | C56:3 | 1.84 | 0.0058 |
| | C56:2 | 2.15 | 0.0069 |
| | C58:10 | 5.38 | 0.0004 |
| | C58:9 | 2.94 | 2.05E−06 |
| | C58:8 | 2.43 | 1.13E−07 |
| | C58:7 | 1.93 | 6.78E−10 |
| | C58:6 | 2.42 | 1.40E−09 |
| | C58:4 | 2.70 | 1.62E−05 |
| | C58:3 | 2.15 | 0.0001 |
| | C58:2 | 1.37 | 0.0077 |

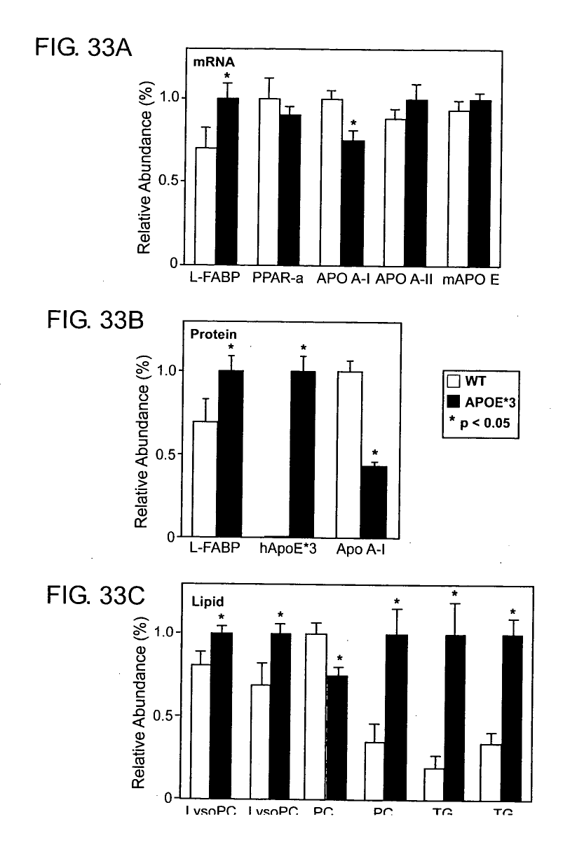

Discussion. As highlighted in FIGS. 33A-33C, the comprehensive systems analysis based on differential genomic, proteomic, a metabolomic profiling yielded a number of novel observations that distinguish the APOE*3-Leiden transgenic mouse from wild type controls under conditions where the mice display essentially no clinical indications of disease. Following PCDA clustering analysis and identification of differentiating factors, the relative abundance of each biomolecular component type, mRNA, protein, and lipid, was calculated and is shown in FIGS. 33A, 33B, and 33C, respectively. Values represent the mean±SEM for n=4-5 separate animals (* p<0.05). Taken individually, each of these entities may serve as a biomarker of an altered metabolic state that predisposes a subject to hyperlipidemia and atherosclerosis.

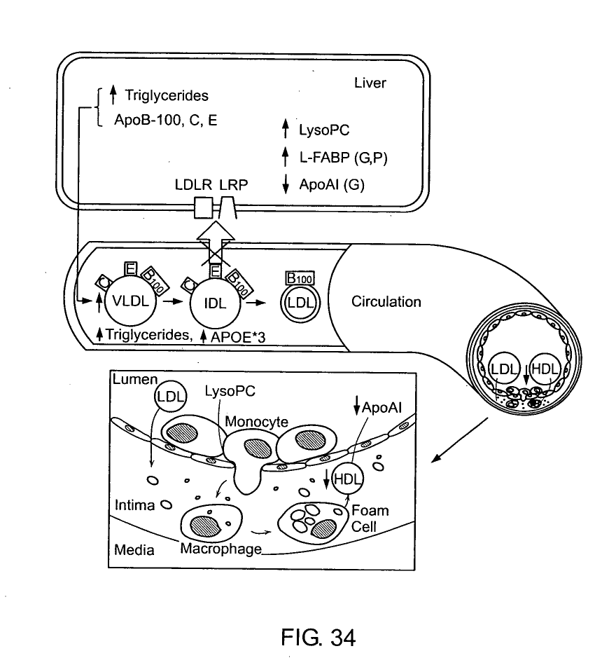

Key species in atherosclerosis identified as early markers of disease in the APOE*3-Leiden mouse are illustrated in FIG. 34. In humans, the APOE*3-Leiden mutation gives rise to a dysfunctional apolipoprotein E variant that is has reduced affinity for the low-density lipoprotein receptor (LDLR). Similarly, APOE*3-Leiden transgenic mice also develop hyperlipidemia and are susceptible to diet-induced atherosclerosis. Early markers of pathology that were found via systems biology in young mice that were reared on a normal chow diet are indicated with arrows (upward pointing denotes up-regulation in the transgenic, while downward pointing denotes down-regulation in the transgenic). These markers include Apo AI and L-FABP mRNA and protein, and a variety of lipid molecules. For example, lipoprotein-associated phospholipase A₂ (which is also described as platelet activating factor acetyl hydrolase) is an enzyme that catalyzes the generation of LysoPC from PC in circulation and has been identified as a risk factor for heart disease. [Packard et al., N. Engl. J. Med. 343 1148 (2000).] LysoPC contributes to early pro-inflammatory events that contribute to pathogenesis, where they increase monocyte adhesion and chemotaxis during fatty streak development. In the present study, two LysoPC compounds that are elevated in the livers of APOE*3-Leiden transgenic mice were identified, suggesting that early inflammatory events in the liver may play a role in the pathogenesis of atherosclerosis.

The apolipoproteins and L-FABP constitute a second macromolecular group of biomarkers. Apolipoprotein AI (ApoAI) is significantly lower in the plasma of APOE*3-Leiden mice compared to wild type controls. Here, mRNA transcripts for this apolipoprotein were found to be lower in the liver, bolstering the previous observation and therefore supporting a role for lowered ApoAI and HDL levels as contributing factors to predisposition to disease.

Evidence for elevated L-FABP was also provided by both genomic and proteomic analyses. ApoE-deficient mice that were also deficient for adipocyte fatty acid binding protein, aP2, were protected against atherosclerosis via a mechanism involving impaired macrophage function. [Makowski et al., Nat. Med. 7, 699 (2001).] L-FABP is member of the same family of intracellular fatty acid binding proteins. It is believed to play a role in transcriptional regulation by acting as a shuttle for ligands of PPARα. [Wolfrum et al., Proc. Natl. Acad. Sci. USA 98, 2323 (2001).] In humans, ApoAI expression is transcriptionally regulated by PPARα. Of particular interest, the results of the present study show an uncoupling of the relationship between L-FABP and PPARα-mediated ApoAI expression, since L-FABP levels were elevated, PPARα levels were unchanged, and ApoAI expression was lowered. These results therefore suggest that an additional, but essential, factor is absent or down regulated. It is intriguing to speculate that this factor might be a particular ligand for PPARα.

In conclusion, we have shown that the results of systems biology approach of profiling at the mRNA, protein, and lipid levels has uncovered a number of novel biomarkers for early predisposition of APOE*3-Leiden transgenic mice for development of atherosclerosis. Taken collectively, collections of such entities may constitute unique, composite biomarkers that allow for greater precision in differentiating multifactoral diseases. This systems biology approach has enabled the elucidation of interconnected relationships among several of these biomarkers and has provided insight into both the mechanism of disease as well as avenues for therapeutic intervention.

### EXAMPLE 4

### Systems Biology Approach: Multiparallel Analysis of the ApoE3-Leiden Transgenic Mouse Model

The results of a systems biology analysis of pathogenetic processes in a complex mammalian hyperlipidemia and atherosclerosis disease model are presented below. A platform integrating proteomic and metabolomic analyses and quantitative differentiating disease factors underlying a transgenic system are described. To gain insight into a multifactorial disease such as hyperlipidemia and atherosclerosis, a systems biology approach to profile protein and metabolite constituents in whole plasma of ApoE*3-Leiden transgenic mice was used. The results confirm known lipid metabolism processes, and elucidate novel differences at the lipoprotein and lipid levels in the transgenic disease model.

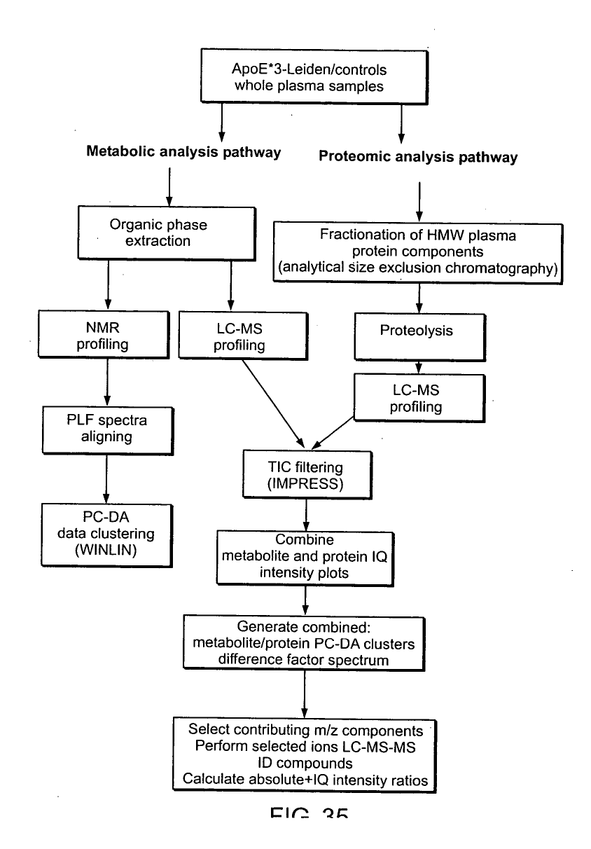

The overall approach to systems analysis, a whole plasma parallel proteo-metabolic profiling scheme, applied in this study is schematically outlined in FIG. 35. Whole plasma, lipid, and protein fractions from ApoE*3-Leiden and control mice were analyzed by NMR and MS. Both metabolic and protein data sets were filtered through the IMPRESS algorithm and clustered simultaneously using WINLIN statistical software as described in the text. Separation and spectroscopic analytical methods, such as HPLC, NMR and LC/MS, were combined with powerful statistical pattern recognition algorithms, such as discriminant analysis, to rapidly cluster and identify biochemical constituents in plasma of control vs. genetically perturbed animals. The results show major (>2-fold) and less obvious, but statistically significant (p<0.05 t-test) differences at the protein and metabolite levels.

Animals. APOE*3-Leiden transgenic mouse strains were generated by microinjecting a twenty-seven kilobase genomic DNA construct containing the human APOE*3-Leiden gene, the APOC1 gene, and a regulatory element termed the hepatic control region that resides between APOC1 and APOE*3 into male pronuclei of fertilized mouse eggs. The source of eggs was superovulated (C57B1/6J×CBA/J) F1 females. Transgenic founder mice were further bred with C57B1/6J mice to establish transgenic strains. Transgenic and non-transgenic littermates of F21-F22 generations were used in these experiments. All mice were fed a normal chow diet (SRM-A, Hope Farms, Woerden, The Netherlands) and sacrificed at nine weeks, at which time plasma tissue samples were taken and frozen in liquid nitrogen. The samples from each individual were then subdivided for separate protein and metabolite analyses.

Plasma lipoprotein profiling. Plasma from 9-week old mice that were kept on regular chow diet (SRM-A, Hope Farms, Woerden, The Netherlands) was fractionated by size exclusion chromatography through a Super SW3000 TSKgel column (Tosoh Biosep, Tokyo) on an LC Packings chromatography system (Dionex, Marlton, N.J.). Total protein concentration for each sample was determined by the Bradford assay and 10 μL of whole plasma normalized to the lowest concentration was injected and eluted isocraticly in 20 mM Bis-Tris Propane, pH 6.9; 100 mM NaCl at 50 μL/minute. Base-resolved peaks corresponding to molecular weight ranges of greater than 300 kD were collected as discrete fractions. Proteins were digested, thermally denatured and reduced in 100 mM ammonium bicarbonate, 5 mM calcium chloride and 10 mM dithiothreitol at 75° C. for 30 minutes, alkylated with 25 mM iodoacetamide at 75° C. for 30 minutes, and then digested with 0.3% (w/w trypsin/protein) for 24 hours at 37° C.

Protein LC/MS analysis. Liquid chromatography-mass spectrometry (LC/MS) was performed using an LCQ DecaXP (ThermoFinnigan, San Jose, Calif.) quadrupole ion trap mass spectrometer system equipped with an electrospray ionization probe. The LC component consisted of a Surveyor autosampler and quaternary gradient pump (ThermoFinnigan, San Jose, Calif.). Samples were suspended in mobile phase and eluted through a Vydac low-TFA C18 column (150×1 mm, 5 μm) (GraceVydac, Hesperia, Calif.). The column was eluted at 50 μL/minute isocraticly for two minutes with Solvent A (water/acetonitrile/acetic acid/trifluoroacetic acid, 95:4.95:0.04:0.01, vol/vol/vol/vol) followed by a linear gradient over 43 minutes to 75% Solvent B (water/acetonitrile/acetic acid/trifluoroacetic acid, 20:79.95:0.04:0.01, vol/vol/vol/vol). The electrospray ionization voltage was set to 4.25 kV and the heated transfer capillary to 200° C. Nitrogen sheath and auxiliary gas settings were 25 and 3 units, respectively. For quantification of tryptic peptides, the scan cycle consisted of a single full scan mass spectrum acquired over m/z 400-2000 in the positive ion mode. Data-dependent product ion mass spectra (MS/MS) were also acquired for peptide identification using the TurboSEQUEST algorithm (ThermoFinnigan, San Jose, Calif.) in conjunction with NCBInr, Swissprot and MSDB data base searches using MASCOT search algorithm (Matrix Science).

Metabolite analysis. The mouse plasma samples were prepared for global lipid and metabolite analysis by adding 0.6 mL of isopropanol to 150 μL of whole plasma followed by centrifugation to precipitate and remove proteins. A 500 μL aliquot of the supernatant was concentrated to dryness and redissolved in 750 μL of MeOD prior to NMR analysis. To prepare samples for LC/MS, 400 μL of water was added to 100 μL of the supernatant, and 200 μL of this mixture was transferred to an autosampler for LC/MS.

NMR analysis. NMR spectra were recorded in triplicate in a fully automated manner on a Varian UNITY 400 MHz spectrometer using a proton NMR set-up operating at a temperature of 293 K. Free induction decays (FIDs) were collected as 64K data points with a spectral width of 8.000 Hz; 45 degree pulses were used with an acquisition time of 4.10 s and a relaxation delay of 2 s. The spectra were acquired by accumulation of 512 FIDs. The spectra were processed using the standard Varian software. An exponential window function with a line broadening of 0.5 Hz and a manual baseline correction was applied to all spectra. After referring to the —CD₃ signal of CD₃OD (δ=3.30), line listings were prepared using the standard Varian NMR software. To obtain these listings all lines in the spectra above a threshold corresponding to about three times the signal-to-noise ratio were collected and converted to a data file suitable for statistical analysis applications.

LC/MS analysis. An LSQ Classic (ThermoFinnigan, San Jose) was used to acquire plasma lipid and metabolite component MS spectra. The LC component consisted of a Waters 717 series autosampler and a 600 series single gradient forming pump (Waters Corporation, Milford, Mass.). Samples were injected onto an Inertsil column from (ODS 3, 5 μM, 3 mm×100 mm) protected by an R2 guard column (Chrompack). A 75 μL aliquot of mouse plasma extract was injected twice in a random order. The random sequence was applied to prevent detrimental effects of possible drift during analysis on the results obtained from statistical statistics. The elution gradient was formed by using three mobile phases: (1) (water/acetonitrile/ammonium acetate (1M/L)/formic acid, 93.9:5:1:0.1, vol/vol/vol/vol), (2) (acetonitrile/isopropanol/ammonium acetate, (1M/L)/formic acid, 68.9:30:1:01, vol/vol/vol/vol), (3) (isopropanol/dichloromethane/ammonium acetate (1M/L)/formic acid, 48.9:50:1:0.1, vol/vol/vol/vol). The samples were fractionated at 0.7 mL/minute by a four-step gradient: (1) over 15 minutes going from 30% to 95% buffer B; (2) 20 minute gradient from 95% to 35% B and 60% C with a 5 minute hold at this step; (3) rapid one minute gradient of 35% B and 60% C going to 95 and 0% respectively; and (4) 95% buffer B going back to 30% over 5 minute period.

The electrospray ionization voltage was set to 4.0 kV and the heated transfer capillary to 250° C. Nitrogen sheath and auxiliary gas settings were 70 and 15 units, respectively. For quantification of metabolites, the scan cycle consisted of a single full scan (1 s/scan) mass spectrum acquired over m/z 200-1700 in the positive ion mode.

Data pre-processing NMR. The NMR spectra were aligned manually with WINLIN statistical software package (TNO Pharma, Zeist, The Netherlands).

Data pre-processing LC/MS. The LC/MS data files were converted to NetCDF format using Xcalibur software (ThermoFinnigan). The converted files were evaluated with IMPRESS post acquisition noise reduction and normalization software (TNO Pharma, Zeist, The Netherlands) to obtain a fingerprint spectrum for each of the LC/MS files. The program evaluates each mass trace for its chromatographic quality by assessing its information content. This is performed, after smoothing to remove spikes and by calculating for each mass the entropy of the trace according to Equation 12. Taking the reciprocal value of H and scaling all results to the largest value gives each mass trace a scaled chromatographic quality, or IQ.

PCA and PC-DA analysis. Principal component (PCA) and discriminant analysis (PC-DA) were applied to the fingerprint spectra of the aligned plasma NMR spectra and IMPRESS preprocessed LC/MS spectra. This was done using WINLIN statistical software (TNO Pharma, Zeist, The Netherlands).

Differential metabolic NMR analysis. To evaluate the pattern recognition and clustering methods for metabolite analysis, a dual approach was used, where NMR was utilized as the initial screening method followed by LC/MS, which has been established as a benchmark analytical method for metabolome profiling in a variety of biological systems. [Raamsdonk et al., Nature Biotech. 19, 45 (2001).; Nicholson et al. Xenobiotica 29, 1181 (1999); Fien et al., Anal. Chem. 72, 3573 (2000).] To facilitate NMR data processing, the WINLIN software package was applied to cluster and estimate the degree of variance between the wild type and transgenic data sets. Sufficient differences, based on the preliminary NMR screen, have emerged to warrant further detailed analysis using MS and MS/MS.

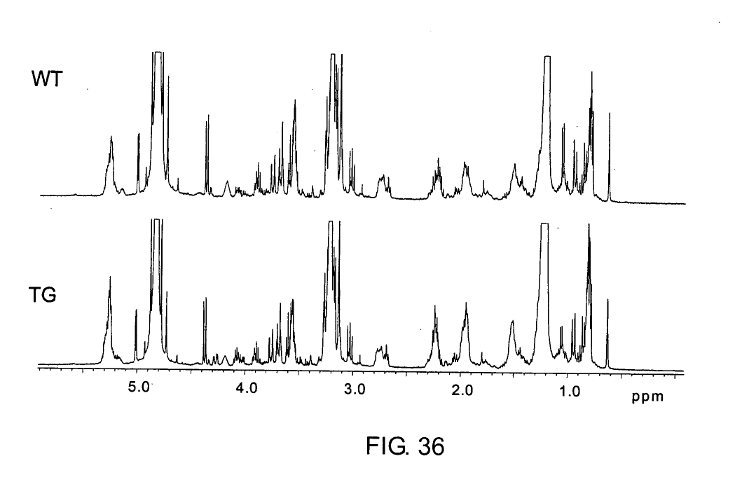

Whole plasma samples from 20 mice (n=10 for each group) were used for global metabolite NMR analysis. For a typical 400 MHz ¹H NMR, 750 μL of deproteinated sample in MeOD were used to generate triplicate spectra, which are illustrated in FIG. 36, for both the wildtype mouse plasma sample (WT) and the Leiden mouse plasma sample (TG). After referring to the —CH₃ signal of MeOD (δ=3.30), line listings were prepared using the standard Varian NMR software. To obtain these listings, all resonances in the spectra above a threshold corresponding to about three times the signal-to-noise ratio were collected and converted to a data file format suitable for statistical analysis applications. The intent for using NMR fingerprinting for initial analysis of plasma metabolite components was not to assign signals to specific compounds, but to establish whether the samples exhibit sufficient clustering and thus warrant a more detailed analysis. Close examination of the NMR data revealed small variations in the resonance position of comparable lines. Variations in the positions of lines are due to the relative concentration of the compounds in the samples and the instrument instabilities, such as the temperature and the homogeneity of the magnetic field, which were corrected for manually. Spectra processed in this manner were imported into the WINLIN statistical analysis tool for discriminant component analysis (PC-DA) clustering.

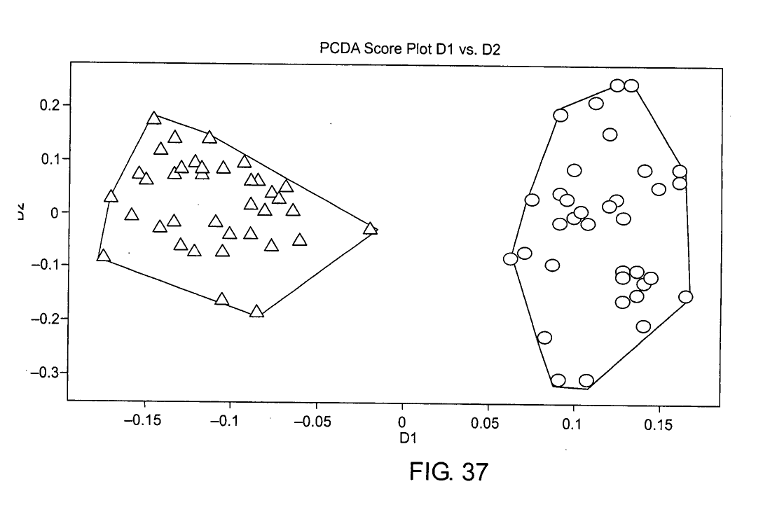

FIG. 37 illustrates a PC-DA score plot showing clustering of NMR data for the Leiden mouse, represented by triangles, and the control mouse, represented by circles. WINLIN allows graphical clustering of results after the data are normalized and subjected to principal component analysis (PCA). Each point within the cluster is spatially positioned to represent one of the triplicate sets of the preprocessed spectra. Concentration intensities from each of the triplicate spectra were used to construct the PC-DA cluster sets. The first step in principal component analysis is the extraction of eigenvectors from the variance/covariance matrix to obtain a number of orthogonal sets of new variables, called principal components, that are optimized in their ability to explain a maximum amount of variance in the original data. In highly correlated data, a few of the top ranking principal components will be sufficient to reproduce the significant variance in the original data set. PCA was applied to reduce the number of features needed to investigate the partial linear fit (PLF) aligned NMR spectra of the control and APOE*3 Leiden mice. Projections of the samples onto the first fifteen principal component axes were then used as starting point for linear discriminant analysis.

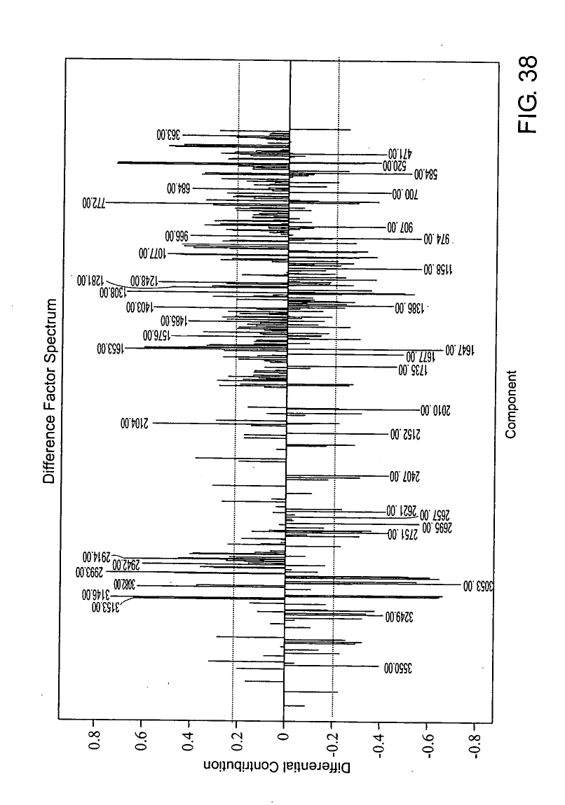

Factor spectra were used to correlate the position of clusters in the score plots to the original features in the spectra by a graphical rotation of the loading vectors. [Windig et al., Anal. Chem. 56, 2297 (1984).] The difference factor spectrum plot, shown in FIG. 38, is characterized by a number of lines representing various metabolic components defined by a range of contribution factors, specifically, ion m/z's that facilitated clustering of transgenic and control mouse populations. The height of the lines above and below the axis of the plot is directly related to the amplitude of the contribution to the overall variance where the factors extending below the axis correspond to higher spectral intensities in the transgenic animals. Since PC-DA separates clusters in a single unique direction, lines projecting below the central axis represent NMR spectral pattern components of higher intensity in the plasma of transgenic mice. The lines extending above the central axis symbolize factors present at higher absolute concentrations relative to the control group.

Factor spectra prepared in directions of maximum separation of the two categories were used to give an insight into the type of metabolites responsible for the separation of the observed categories. Preliminary results based on the PC-DA loading plots point to the δ3.8 ppm-δ4.2 ppm region and the lipid region (δ 1.2 ppm-δ 0.8 ppm) as the primary contributors to quantitative variance between Leiden and control samples.

The limitations of NMR spectroscopy result from the low inherent sensitivity of the technique and from the high complexity and information content of NMR spectra. The sensitivity of the technique is also affected by the minimum threshold concentrations of compounds being detected. Regardless of its limitations, it is clear that NMR based metabolome profiling coupled to pattern recognition technology is a powerful analytical approach for integration of metabolic data into a comprehensive systems-level analysis. In this study however, the purpose of the NMR screen was not to identify specific molecules, but rather to use the method to determine whether a qualitative degree of differentiation between sample populations exists.

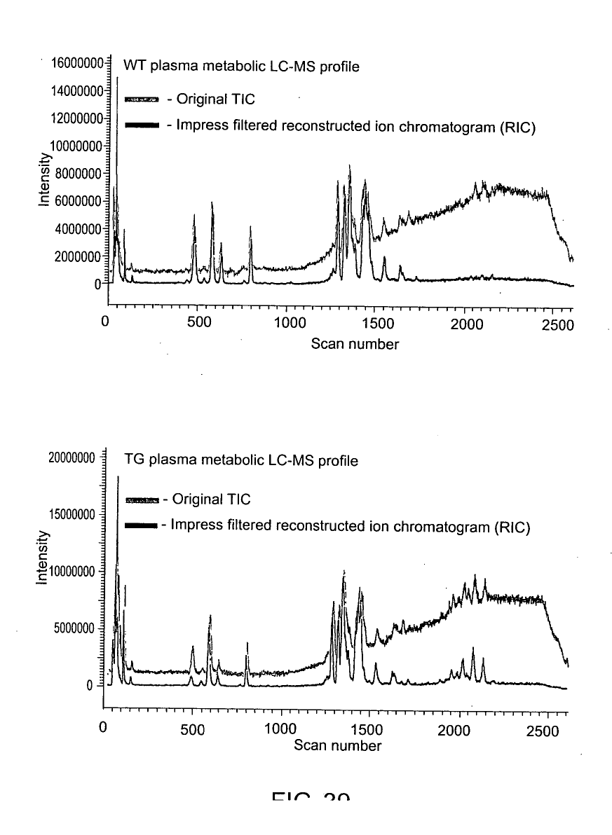

Simultaneous analysis of metabolic and protein components yields expected and novel patterns. Metabolite extracts from plasma of transgenic (n=4) and control (n=4) mice were prepared by the isopropanol precipitation method. Upon addition of 400 μL of water to 100 μL of extract, the samples were subjected to LC/MS analysis. FIG. 39 depicts TICs that were collected using single scan mode over the 400-1700 m/z mass range. To apply statistical statistics to the LC/MS spectra, the raw data files were first converted to NetCDF format and processed using IMPRESS noise reduction and normalization software. The program evaluates each mass trace for its chromatographic quality by assessing its information content. This is performed, after smoothing to remove spikes and by calculating the entropy for each m/z of the trace according to Equation 12. Mass intensities normalized by IMPRESS are assigned a scaled chromatographic quality number, or the IQ. To perform principal component analysis, the IQ based chromatograms in FIG. 39 were imported into WINLIN, and discriminant analysis separation was obtained based on two initial principal component vectors.

The proteomic whole plasma analysis was biased towards fractions containing lipoprotein complexes. This was in line with expectations that most statistically relevant changes associated with the Leiden mutation will occur in this class of proteins, based on the transgenic model selected. Whole plasma samples from the transgenic (n=4) and control (n=4) animals were fractionated by analytical size exclusion chromatography and fractions corresponding to high molecular weight plasma protein component were isolated as described in the experimental protocol. Two major early peaks eluted at 23 minutes and 27 minutes, corresponding to VLDL (fraction 1) and HDL (fraction 2) components of whole plasma, respectively, were used for all subsequent manipulations. Proteins contained in fractions 1 and 2 were treated with trypsin to generate proteolytic peptides.

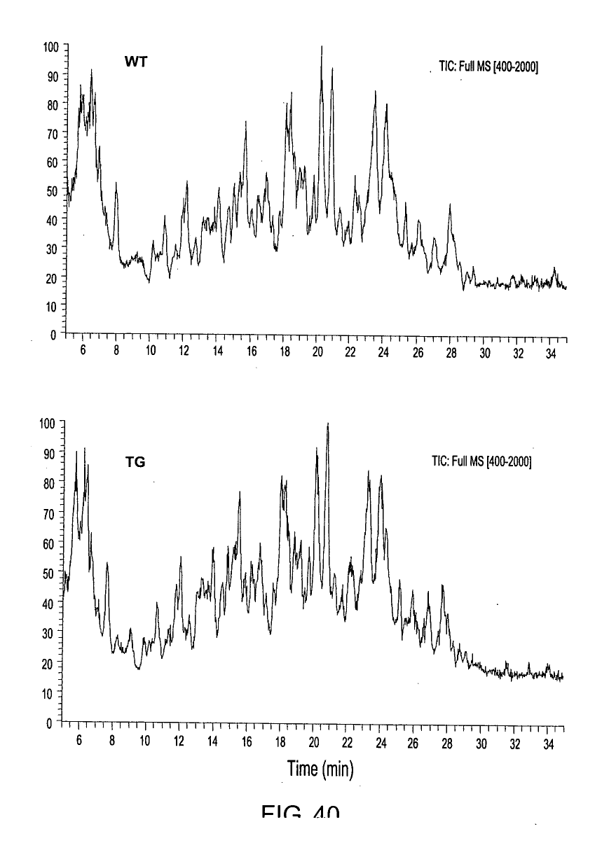

TICs of the VLDL fractions from the MS analysis are shown in FIG. 40 for the wildtype mouse (WT) and the Leiden mouse (TG). MS/MS spectra collected for all eight representative samples were analyzed by TurboSEQUEST to generate hits against NCBI nonredundant, human and mouse databases. The identities of these initial hits were further verified using the MASCOT de novo sequencing and database search tool. The threshold for assigning protein identities was based on the minimal sequence coverage set at 20% of total residue count. The protein MS data were clustered in a way similar to the metabolic component by generating IQ value spectra followed by discriminant analysis.

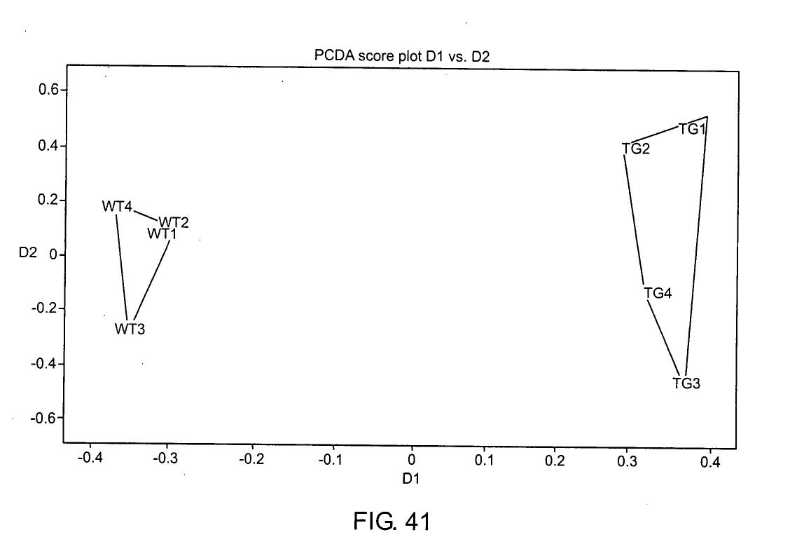

To observe quantitative relationships between metabolic and protein components of plasma, an assembly of concatenated heterogeneous data sets was used. Original individual data sets were integrated separately and IMPRESS quality m/z values from these sets were summed and subjected to the statistical clustering analysis. The resulting score plot, which is illustrated in FIG. 41, shows PC-DA clusters for the wild type (WT) and transgenic (TG) animals generated based on two principal components rotated to achieve maximum separation in D1. Each point represents linear combination of metabolite and protein variance factors (60% of original data set) for the individual animals.

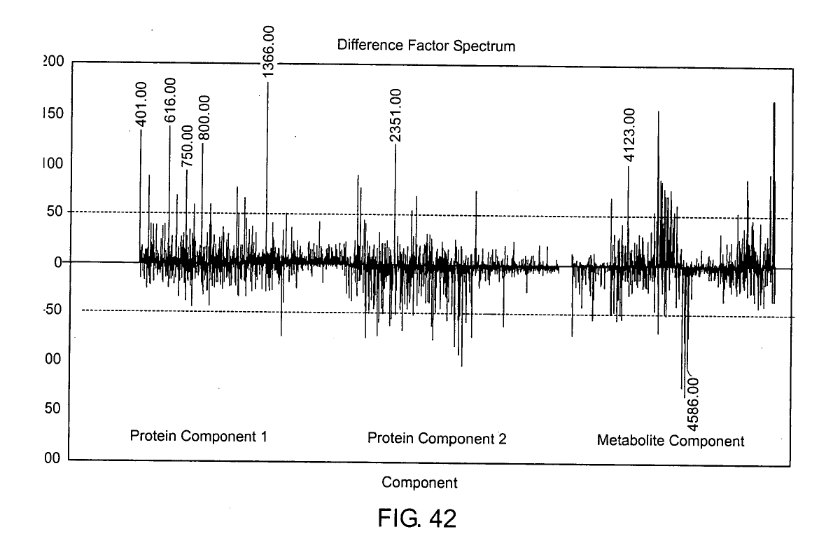

Filtered m/z intensities from metabolite and peptide spectra were organized in a linear fashion in the factor plot, shown in FIG. 42. Linear distribution along the central axis represents protein and metabolite components with calculated bi-directional contributions to variance between the control and transgenic groups. Main positively contributing factors are seen projecting above the nominal cut-off weight of 50. Negative contributors to the overall variance project below the −50 set boundary.

By adding nominal values of 1601 and 3401 to each m/z value in the second protein and the metabolic components, respectively, heterogeneous experimental data was analyzed in parallel, as shown in FIG. 42. Significant contribution intensities were scored based on the factor plot specific threshold parameter, which was set to 50 in this instance. The masses that were found to be major differentiators between the WT and TG data sets were extracted and identified by LC/MS/MS. The combination intensities (raw data and IQ scores) of differentiating factors were measured directly in the LC/MS chromatograms for statistical significance (P<0.05) and calculation of fold change.

The results point to a composite profile that corroborates previous findings with respect to lipoprotein and lipid abnormalities associated with the APOE*Leiden phenotype. [Mensenkamp et al., J. Hepat. 33, 189 (2000); van den Maagdenberg et al., J. Biol. Chem. 268, 10540 (1993); Williams van Dijk et al., Arterioscler. Thromb. Vasc. Biol. 19, 2945 (1999); and Mensenkamp et al., J. Biol. Chem. 274, 35711 (1999).] Specifically, at the protein level we were able to show that human APOE*3Leiden allelic variant is expressed and functionally active in the transgenic animals as evidenced by its incorporation into VLDL (protein component 1 in FIG. 42) and LDL/HDL (protein component 2 in FIG. 42) fractions of plasma derived lipoproteins. Alternatively, murine ApoA1 has been found to be twofold less abundant in the plasma of transgenic mice indicating lower degree of incorporation of the apolipoprotein into the LDL/HDL complexes in these animals.

Although the underlying processes governing HDL metabolism have not been fully defined, HDL levels in plasma have been shown to have inverse relationship with atherosclerosis susceptibility. [Callow et al., Genome Res. 10, 2022 (2000); and Glass and Witztum.] A number of different mechanisms can control HDL plasma. Most prominent factors identified in mouse models that contribute to lowering plasma HDL include defects in apoA1, apoE, phospholipid transfer protein (PLTP) and the overexpression of cholesteryl ester transfer protein (CETP) or scavenger receptor SRB. [Callow et al.; Williamson et al., Proc. Natl. Acad. Sci. USA 89, 7134 (1992); and Wang et al., J. Biol. Chem. 273, 32920 (1998).] Assuming that the Leiden mutation is functionally analogous to a defective APOE allele, it is highly likely that, in the context of the Leiden model, the lower HDL levels are at least partially the result of the ApoE*3 transgene function. One possibility for decrease in total endogenous ApoA1 is the stoichiometric imbalance due to constituent overexpression of the hApoE3 and its preferential recruitment for LDL/HDL assembly.

This study demonstrates the utility of a multilevel approach for characterization of a highly complex system. By generating high content analytical output and comparing integrated principle component factors derived from composite data sets, rapid elucidation of identities and the relative abundances of major lipoprotein metabolism mediators that define ApoE*3-Leiden phenotype was possible. Solely based on a biofluid analysis, this effort represents the first attempt to apply systems biology rationale in a way that unites quantitative proteomic and metabolome data to explain disease. In the future, it will be possible to enhance this approach by including the genomic component in the form of differential transcription analysis of multiple tissues and make it truly global with respect to understanding pleotropic effects of gene perturbations.

### EXAMPLE 5

### Systems biology Approach: Metabolic Disease Study

Summary. The overall goal of this example is to demonstrate molecular analysis and data integration capabilities according to the invention. The general area of medical interest was metabolic disease, and the materials to be analyzed were serum samples from two animal species (rodent and non-human primate) and from human subjects. A subset of each group of rodents (diseased and control) was drug treated. During the initial phase of the project (Phase I), the testor was aware that there were three sample sources (rodent, non-human primate, and human) but was blinded to the details of the grouping of the samples within each species.

The specific objectives of the study were as follows.

Phase I

to undertake metabolite and protein analyses of blinded serum samples from animal and human subjects; and
to group the samples based on the serum metabolite and protein profiles.
   Phase II
after unblinding, to compare the grouping of the samples as determined with the actual sample groups;
to define, for each of the sample types, molecular components (biomarkers) that can be used to differentiate one group of samples from another;
to construct correlation networks for the biomarkers in order to gain insight into the biochemical processes underlying the disease or drug treated phenotypes; and
to determine whether molecular components which differentiate diseased rodents from control rodents are similar to those which differentiate diseased human patients from control human subjects.

Blinded analyses of the metabolite and protein profiles for the rat serum samples revealed four clearly distinct groups that, upon unblinding, corresponded exactly to the actual groups of samples (Diseased+vehicle, Diseased+drug, Control+vehicle, Control+drug). Blinded analyses of the profiles for the non-human primate samples revealed two distinct groups that, upon unblinding, corresponded exactly to the diseased and control groups. For the human samples, blinded analyses of the metabolite and protein profiles revealed different numbers of groups (4 or 2), depending upon the analytical platform employed. Analysis based only on lipid profiles revealed two groups that, upon unblinding, corresponded with 86% accuracy to the diseased patients and with 89% accuracy to the control subjects.

A large number of metabolites and proteins were identified that differentiated between the groups of animal and human serum samples. The relative levels of these biomarkers in the samples provided insight into the biochemical processes underlying the disease or drug response. One of the notable findings was the effect in the diseased rodents of the drug treatment on serum protein levels. A second, distinct finding was the almost identical widespread changes in the levels of over 150 serum lipids in both the diseased rodents and the diseased patients relative to the levels in the corresponding control subjects. As a validation of the rodent model as a model of the human disease, the testor was also able to use the set of serum lipid biomarkers found to correctly classify diseased versus control rodents to distinguish with good precision the diseased patients from the control human subjects.

Introduction. The overall goal of this example was to provide a basis to assess integrated platforms of proteomics, metabolomics and informatics technologies as applied to comparative studies of pre-clinical and clinical serum samples. Serum samples were provided from a drug treatment study in a rodent model of metabolic disease, a comparative study of metabolic disease in human subjects, and a study of a related condition in non-human primates. The project was divided into two phases. In Phase I, the testor was blinded with respect to sample information and performed comparative quantitative profiling of metabolites and proteins using a combination of NMR and MS techniques. Informatics methods such as unsupervised clustering analyses were applied to the data to determine if the experimental groups could be accurately discriminated. At the conclusion of Phase I, the data was unblinded, and it was revealed that the methods used had determined groups with a high degree of accuracy. The emphasis of the second phase was identification of metabolites and proteins that contributed to the differentiation of the four experimental groups within the rodent drug treatment/disease study as well as a determination of the extent to which individual molecular species are correlated with one another. In addition, correlations between diseased and control human subject groups and their rodent-model counterparts were explored to reveal similarities and dissimilarities between the human disease and the animal model. This Example highlights only certain results in order to exemplify the invention and its techniques.

Sample information. In Phase I of the study, the testor was blinded with respect to whether the samples were from unaffected (normal) or affected (diseased and/or drug-treated) subjects. Unblinding of the sample information was done prior to Phase II. The experimental groups and numbers of samples are listed below.

A. Drug treatment study in a rodent model of metabolic disease: A total of 32 serum samples (600 μL each) from a drug treatment study where a therapeutic drug was administered to diseased rodents and non-diseased rodents (control) were subdivided as follows.
n=8 control treated with vehicle
n=8 control treated with drug
n=8 diseased treated with vehicle
n=8 diseased treated with drug

B. Comparative study of metabolic disease in human subjects: A total of 42 serum samples (300-400 μL per sample) from individuals diagnosed with metabolic disease and controls were subdivided as follows.
n=14 Subjects diagnosed with metabolic disease
n=28 Controls

C. Disease study of non-human primates: A total of 24 serum samples (300-850 μL per sample) from non-human primates were profiled.
n=13 Normal non-human primates monkeys
n=12 Diseased non-human primates monkeys

Methods utilized—Analytical profiling. The approach in the Example to differential proteomics and metabolomics employs several distinct analytical methods that enable the quantitative profiling of a wide range of molecular components. These methods utilize either NMR or MS as analytical endpoints. Profiling platforms have been optimized taking into account robustness, reproducibility, sensitivity, and dynamic range and are designed to survey molecules that may span orders of magnitude in abundance as well as a range of biochemical classes. Each platform has the capacity to profile many components (hundreds to thousands) within a single analysis, and software tools were used to facilitate the extraction of quantitative information for integration into computational and informatics analyses. Methods applied in this study are listed below.
1. Protein LC/MS: allows profiling and identification of peptides and proteins.
2. CPMG NMR: enhanced NMR measurement of low molecular weight metabolites.
3. Diffusion-edited NMR: enhanced measurement of lipoprotein-associated metabolites.
4. Lipid LC/MS: optimized for profiling of lipids and non-polar metabolites.

Methods utilized—Data processing. The resultant NMR spectrum or LC/MS chromatogram obtained from a profiling experiment may contain many hundreds of peaks that represent the relative abundance of hundreds of molecules. Data processing software tools are used to enable the extraction of this information from each data file as well as the comparison of measured peak intensities across the sample set. As described above, typically, data processing steps include peak detection and measurement of relative intensities (peak integration), an “alignment” step to compensate for minor differences in peak position that might occur from one sample analysis to another (i.e., small differences in NMR chemical shift or LC/MS retention time for a particular peak), and assignment of an identifier (or index number) to each peak so that it might be compared across samples.

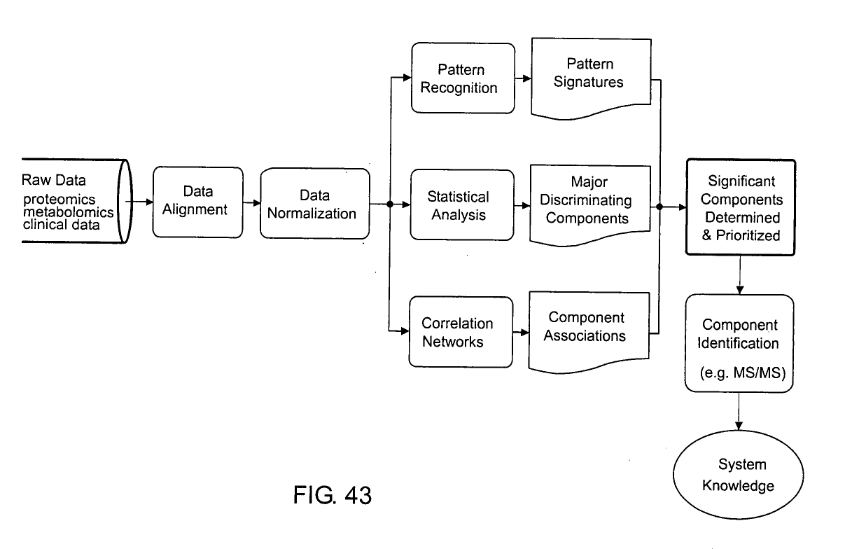

Methods utilized—Data analysis. The data were analyzed using several different statistical approaches: (1) unsupervised clustering of samples (including COSA hierarchical clustering), (2) univariate statistics to determine peaks that are different between groups of samples, and (3) correlation network analysis to identify correlations between individual components of metabolite and protein sets for all samples. In addition, some preliminary data analyses with a support vector machine (SVM) classifier for the purpose of classification were undertaken. FIG. 43 is a schematic representation of the data analysis workflow. Elements of the data analysis process are listed below in the order they are performed.
1. Data Normalization: adjusting for platform-specific variation within the dataset.
2. Application of exploratory unsupervised clustering methods:
   COSA
   Principal Components Analysis
   K-Means Clustering (human samples only)
   Neural network (human samples only).
3. Peak selection for identification: determine significant, discriminating peaks by means of univariate statistical methods (pairwise, two-tailed t-tests) and prioritize for identification.
4. Correlation Networks: determine statistical correlations among pairs of peaks.
5. Data Visualization: use software tools to incorporate database information with the experimentally generated data

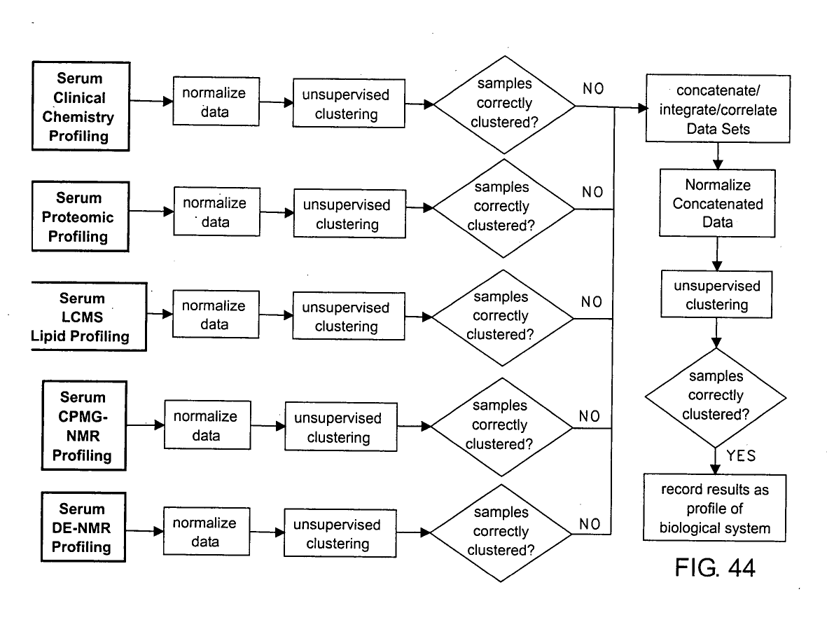

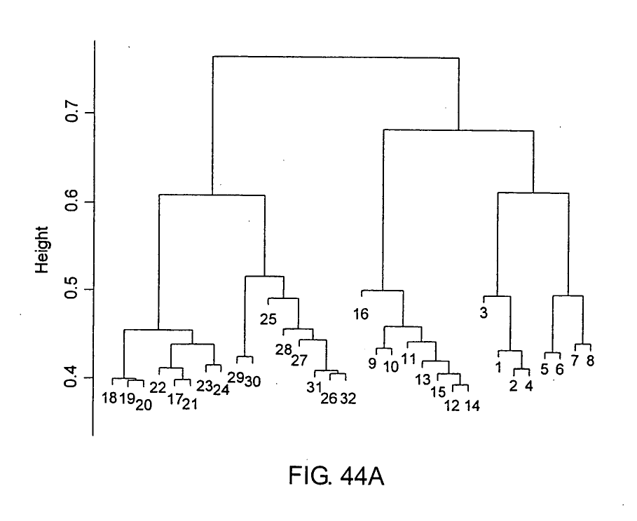

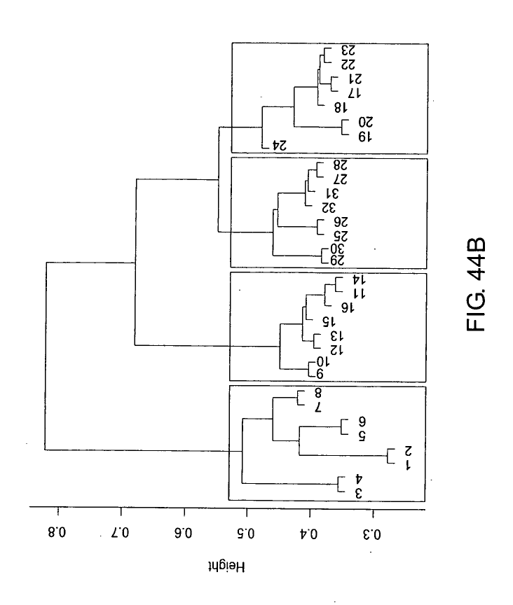

Results and discussion for the rodent model of metabolic disease regarding analyses of serum samples—Unsupervised clustering. Initial analyses focused on unsupervised clustering of data collected from blinded rodent serum samples. Unsupervised clustering is a statistical method that attempts to group samples with no foreknowledge of sample classification or the number of distinct groups in the collection of samples. An outline of the work flow is provided in FIG. 44. In general, multiple data sets from multiple analytical platforms were normalized and clustered. To the extent an individual data set does not correctly or distinctly cluster, the multiple data sets can be concatenated (i.e., combined and/or correlated) for further clustering analysis. In this Example, although certain of the individual data sets showed appropriate clustering, the data sets were concatenated and/or integrated and/or correlated to obtain an even more robust analysis. The concatenated data was normalized and clustered, and the results were recorded as a profile of a biological system.

Data collected from all individual platforms resulted in clustering of blinded serum samples into distinct groups, the only difference between the platforms being the number of clusters formed. Clustering into four groups was observed with both the protein and lipid platforms. These four groups that were ultimately identified consisted of samples 1-8,9-16, 17-24, and 25-32.

The clustering of the LC/MS proteomic data (i.e., a single analytical platform) is illustrated in FIG. 44A. FIG. 44A is an example of the COSA clustering analysis of rodent serum proteomic LC/MS analysis, after data alignment and normalization. In this analysis, the 2,977 peaks that appeared in at least 28/32 rodents (>87% of the samples) were used for clustering. Data obtained from the other metabolite platforms, CPMG NMR and Diffusion-edited NMR, clustered the samples into fewer groups but the divisions were consistent with the groups found during the lipid and protein analyses.

FIG. 44B shows a more robust representation of the four groups (as described above). FIG. 44B is the result of COSA clustering applied to combined data from all platforms. Clustering using CPMG NMR data only revealed three clusters while using DE NMR data only revealed two clusters (not shown). Combining data from proteomics, lipid LC/MS, CPMG NMR and DE NMR (4851 variables total) yielded four clear groups. The groupings were consistent with the results of the individual treatments of the proteomics data and the lipid profiling data.

Unblinding the samples revealed that groups delimited using these methods corresponded exactly to the different rodent cohorts as summarized in Table I below.

TABLE I
Sample Identification Provided After Cluster Analysis

| Sample ID | Cohort |
|---|---|
| 1-8 | diseased rodents treated with vehicle (DISveh) |
| 9-16 | diseased rodents treated with drug (DISdrug) |
| 17-24 | control rodents treated with vehicle (CONveh) |
| 25-32 | control rodents treated with drug (CONdrug) |

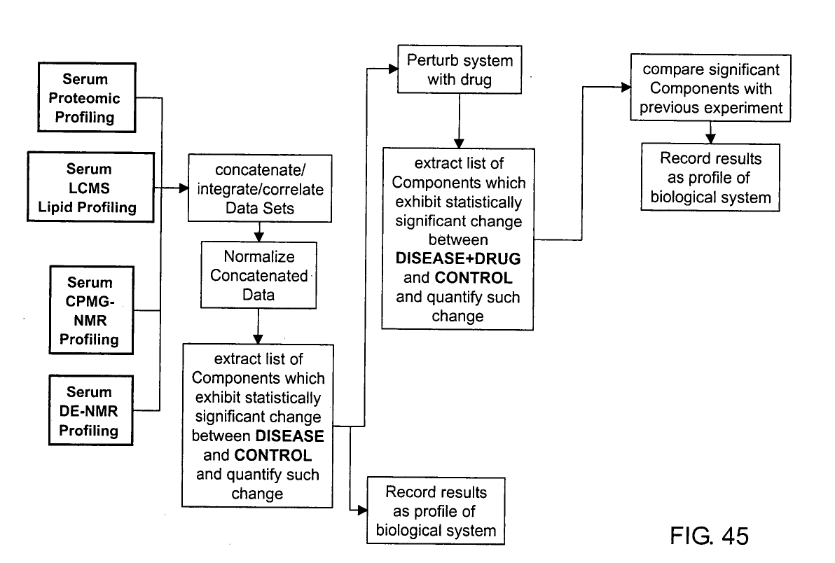

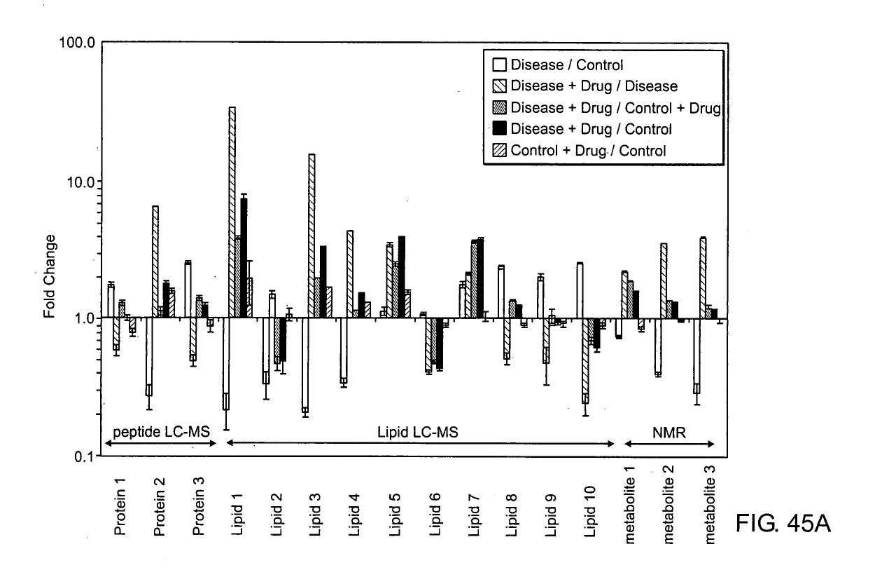

Results and discussion for the rodent model of metabolic disease regarding analyses of serum samples—Metabolite and peptide peak identification. Univariate statistical methods were applied to the peaks profiled in Phase I to select, for subsequent identification, those peaks which exhibited differing abundances among the four groups of rodents. The primary statistical analysis consisted of a pairwise t-test with a significance level α=0.05. The workflow for this analysis is outlined in FIG. 45. In general, multiple data sets from multiple analytical platforms were concatenated, integrated, and correlated, and then normalized. Statistically different components between the disease and control groups were extracted, and the difference was quantified. Then, the system was perturbed by administering a drug to the diseased group, and a similar analysis was undertaken to determine the differences between the treated and control groups. Finally, all of the components identified were compared between the two experiments to obtain a profile of the biological system.

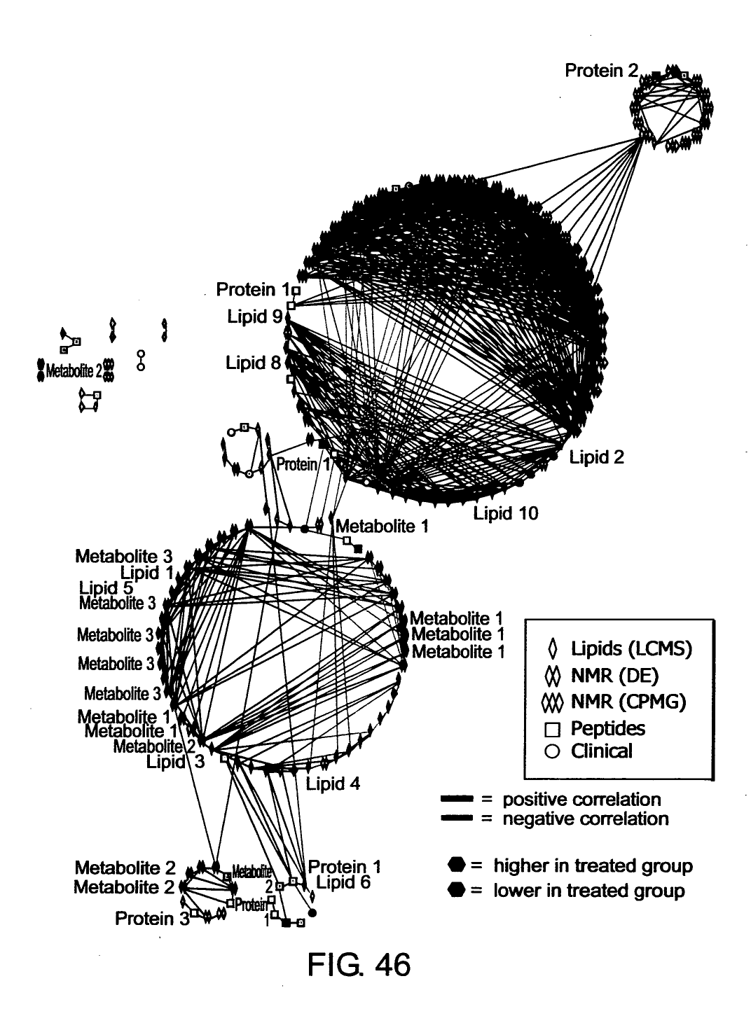

A representative excerpt showing differences observed among metabolites and peptides is shown in FIG. 45A. (These components may also be observed in the correlation network analysis (FIG. 46) where they display correlations among themselves as well as with other identified peptides and metabolites.) By viewing the data in this representation, one can see, for example, that levels of two serum proteins (Protein 1 and Protein 2) were found to be differentially and oppositely regulated between diseased and control rodents (vehicle treated), and that treatment with drug essentially lowers diseased Protein 1 levels to that of the control animals while increasing Protein 2 to levels approximately two-fold higher than the controls. Another interesting observation is the differential effect of drug treatment on select lipid levels.

Note that, for each molecular component, the results are presented in the order below.
1. diseased+vehicle/control+vehicle . . . Effect of disease.
2. diseased+drug/diseased+vehicle . . . Effect of drug treatment on disease state.
3. diseased+drug/control+drug . . . Comparison of drug-treated disease with treated control.
4. diseased+drug/control+vehicle . . . Comparison of drug-treated disease with untreated control.
5. control+drug/control+vehicle . . . “Side effect” of drug.
   This is the order of presentation for all analyses of the rodent serum samples throughout the Example for the instances where all five comparisons have been made.

Results and discussion for the rodent model of metabolic disease regarding analyses of serum samples—Correlation network analysis. In addition to changes in component abundance levels between groups, the examination of correlations between and among individual components is useful to reveal important relationships among the various components studied. Such a correlation analysis is complementary to abundance level information, and often provides information about the biochemical processes underlying the disease or dug response.

FIG. 46 is a representative correlation network derived from the proteomic, metabolomic and clinical chemistry data in the pairwise comparison of the eight diseased drug-treated rodents and the eight diseased vehicle-treated rodents (drug effect on disease state). As can be seen in the legend, the components (or ‘nodes’) of the network are the various proteins, metabolites or clinical chemistries measured by the various platforms. All of the nodes in this figure, and in figures similar to this one, are components which have: (i) been identified, and (ii) exhibited a fold-change greater than ±15% with p<0.05.

There are a number of independent levels of information displayed in this type of correlation network. First, the particular shape of a node represents the platform that was used to measure the component. For example, in FIG. 46, the square shaped nodes are peptides which have been measured and identified (i.e., sequenced and validated) by mass spectrometry. Second, the shading of a given node reflects the abundance difference in the sera of the two groups being compared; this is a normalized group mean difference. Third, the lines between pairs of nodes represent correlations in which the Pearson coefficient is between 0.80 and 1.00, or −0.80 to −1.00. Negative correlation values are presented as light lines, while positively correlated components are connected visually by dark lines in the graphical representation. Generally speaking, two components which are positively correlated reflect a statistically significant mutual behavior characterized by a change in one component being concomitantly related to a similar change in the second component, across all samples in the group. A trivial example may be pairs of peptide components from the same protein which behave similarly, or two NMR resonance components from the same molecule. Biochemically relevant correlations may also be observed, such as between metabolites that are part of the same biosynthetic pathway or between entities that are components of the same macromolecular structure. An example of this type of correlation is shown in FIG. 46, where the Protein 2 peptide is highly positively correlated with a number of lipid components in the serum; this high degree of correlation suggests that these lipids may share the same lipoprotein origin as Protein 2 in serum. Negative correlations may, for example, arise between components that are part of the same pathway, but where they might be separated by a point of enzyme inhibition or substrate limitation. In addition, components that fall past committed biosynthetic branch points may show negative correlations with one another.

The overall topology of the structure is what is referred to as self assembling and reflects clusters of components which are highly inter-correlated. Those nodes which are close to one another reflect a particularly high density of mutual correlation. The topology is generated in an unsupervised and automated fashion.

By investigating such structures, a number of interesting observations become apparent. For example, it is seen that Lipid 2 is higher in abundance upon treatment (the node is at approximately 4 o'clock in the largest circular structure), and furthermore it is negatively correlated with many other lipid components. It should be understood that this figure is illustrative of the principles and techniques of the invention; it is one of many such correlations that are possible.

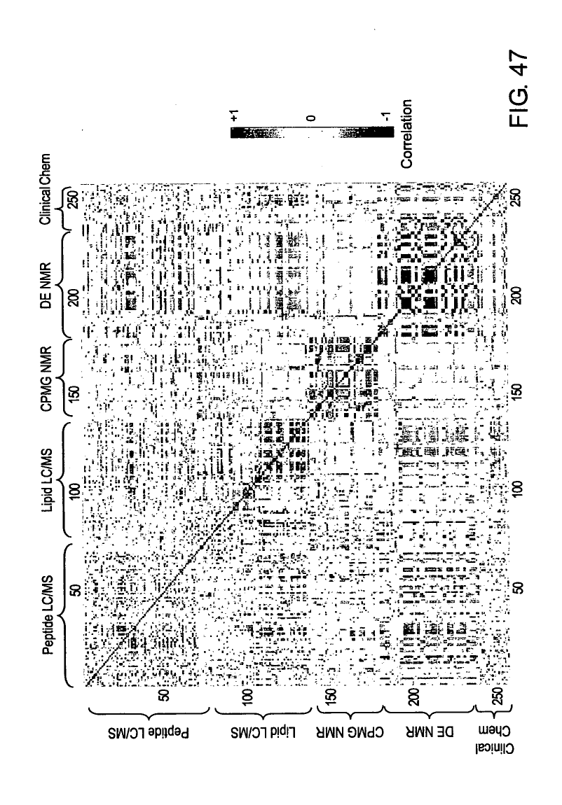

Results and discussion for the rodent model of metabolic disease regarding analyses of serum samples—Heat plot analysis. An alternate view of the correlation information for the comparison of diseased drug-treated and diseased vehicle-treated groups is shown in FIG. 47. This “heat plot” shows an array of correlation coefficients calculated for each pairing of identified metabolite and peptide peaks. The color of the off-diagonal spot for a pair of component peaks corresponds to the sign of the correlation coefficient between the peaks (either positive or negative), while the color intensity is proportional to the magnitude of the correlation.

Though complex, this visualization enables a rapid inspection of the complete array of correlations. When the components are grouped according to analytical method as shown in FIG. 47, correlations between different component classes are apparent. For example, the off-diagonal area that lines up with peptides of index numbers of 22-32 and lipids of index numbers 110-140 shows regions of both high positive and high negative correlations. In this case, the positively correlated peptides (22-26) are from Protein 1 while the lipids are triglycerides. Note that fold-change information is not represented in FIG. 47; the shade scale represents the Pearson correlation coefficient.

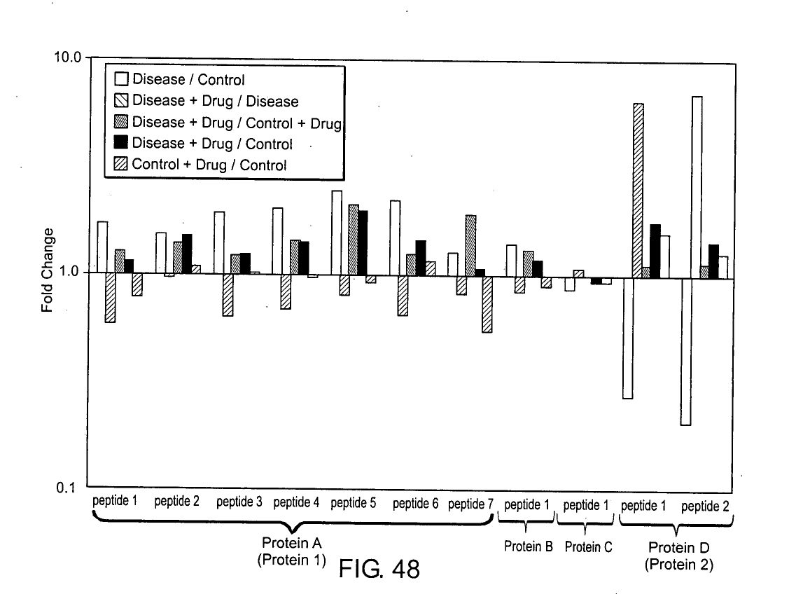

Results and discussion for the rodent model of metabolic disease regarding analyses of serum samples—Rodent protein ratios. Certain proteins play an integral role in lipid metabolism. It is therefore not surprising that differences in the levels of peptides associated with some of these proteins are found in the different sample cohorts examined as part of this study. FIG. 48 illustrates the differences in four such proteins, Protein A (Protein 1), Protein B, Protein C and Protein D (Protein 2), represented as ratios between different groups. Six tryptic peptides were observed from Protein A, one from Protein B, one from Protein C and two from Protein D. The plot in FIG. 48 shows ratios between groups based on the means of the peak intensity values within each group (after normalization and scaling). It is apparent that significant fold changes exist between the different groups. Particularly striking are the Protein D ratio changes between diseased rodents treated with drug and diseased rodents treated with vehicle as well as between the diseased rodents treated with vehicle and the control subgroup of rodents treated with vehicle.

Results and discussion for the metabolic syndrome study regarding analyses of human serum samples—Unsupervised clustering. Unsupervised clustering was applied to the human data derived using all individual platforms, protein, lipid, and NMR. As mentioned above for the rodent model of metabolic disease, this allows grouping of samples with no foreknowledge of sample classification or the number of distinct groups. COSA analysis of the peptide data grouped the samples into four weak clusters. Clustering using the NMR Global metabolite data split the samples into two groups. Once the sample information was unblinded it was apparent that these groupings did not correspond to the diseased vs. control cohorts.

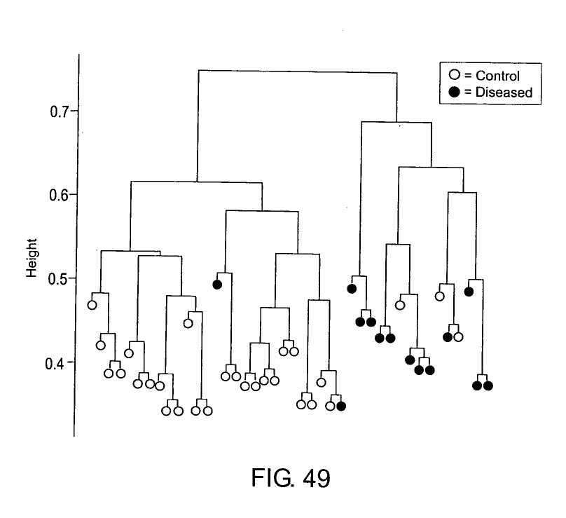

In contrast, COSA analysis of lipid data suggests two clusters (FIG. 49). The COSA distance clustering used 779 human LC/MS lipid peaks. These clusters correspond to the diseased patients with 86% accuracy (12/14) and the control subjects with 89% accuracy (25/28). Multivariate analysis indicated that lipids were the strongest discriminator between diseased and control samples.

The lack of strong clustering in 2 out of the 3 platforms indicates that clustering is dominated by other factors such as medications, gender, age or environment. Given these weak clusters derived using COSA for some of the platforms, other clustering techniques, such as K-Means and neural networks, were investigated using the same data set. These techniques gave results similar to COSA, with the exception of a few samples at the boundaries between groups.

Results and discussion for the metabolic syndrome study regarding analyses of human serum samples—Metabolite and peptide peak identification. As was seen in the rodent study, potentially interesting peaks can be found by highlighting those that differ significantly in level between sample types. For the purpose of this study, the human samples were first divided into the two groups (14 disease patients and 28 control subjects). A two sample t-test was performed for each peak to test for mean differences between the two groups, and this resulted in a list for peaks submitted for identification.

For the lipid platform, a subset of peaks that exhibited differences between diseased patients and control subjects was identified using a reference database as well as targeted MS/MS methods. In general, upon peak identification, it was found that the levels of certain lipid molecules in diseased patients were significantly different from the levels of these lipids in control subjects. Interestingly, as seen in the rodent/human comparison study below, many of these lipid levels are also significantly different in diseased rodents compared to control rodents.

Additionally, a list of human proteins was identified as part of this study using the “shotgun” tandem mass spectrometry (MS/MS) method. There was no overlap between the set of peaks which were selected during the MS profiling stage, for sequencing by shotgun MS/MS, and the set of peaks which exhibited statistically significant level differences between the two groups of human samples serum.

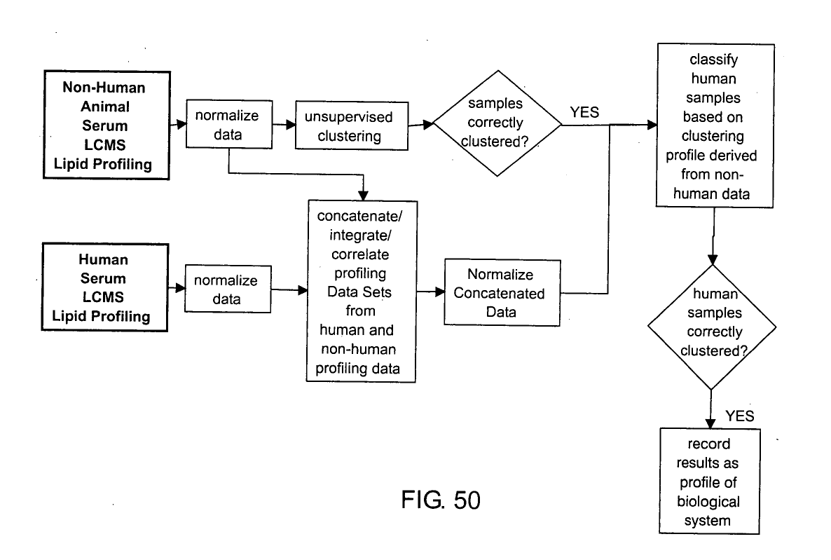

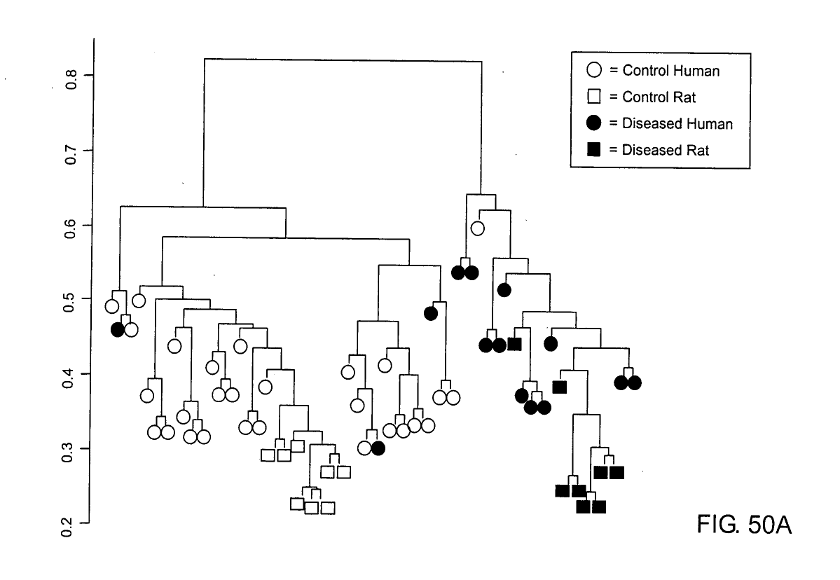

Results and discussion for the comparison of rodent samples with human samples. In this portion of the study, the objective was to compare the lipid components in the serum from diseased vehicle-treated and control vehicle-treated rodents to the corresponding lipids in the serum from diseased and control humans. No drug treatment groups were involved in these analyses. The data from the LC/MS serum lipid platform were used, specifically the 571 LC/MS peaks common to both species. FIG. 50 shows the workflow for this analysis.

In this framework, two issues were addressed. The first issue concerned the accuracy in clustering and classifying human samples based on rodent measurements, and the second issue regarded a comparison across the two species of lipid abundance changes and correlations.

Results and discussion for the comparison of rodent samples with human samples—Clustering and classification. Among the 571 peaks that were common to both species, in 366 there were significant mean changes between the two rodent groups (at a significance level of 0.05 and using two-tailed pairwise t-tests). As an exploratory step, this set of 366 peaks was used to determine whether there were natural clusters in the data comprised of the diseased humans together with the diseased vehicle-treated rodents and the control humans together with the control vehicle-treated rodents. The results of this analysis are shown in FIG. 50A. Specifically, the results of a COSA analysis of human serum samples, in which the input data set used for classification consisted of 366 lipid peaks chosen from the diseased rodent model, is shown. The figure reveals two main groups, corresponding well to the diseased and control samples: 27 of the 28 control humans and all 8 control rodents belong to one group, and 11 of the 14 diseased human and all diseased rodents belong to the second group. It is concluded from this analysis that if the diagnosis of the humans was not known, it could deduced with high accuracy by inspecting the clusters formed in the two rodent groups.

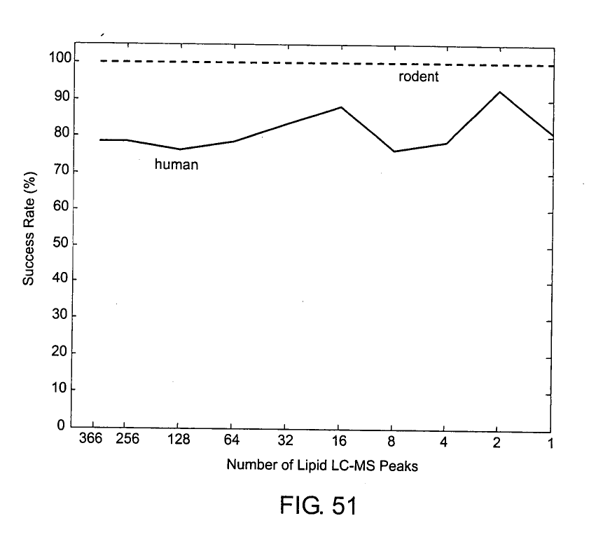

For classification purposes a support vector machine (SVM) linear classifier was used in which the 366 rodent lipid measurements served as the model building set and the corresponding 366 human lipid measurements as an independent test set. The percentage of human samples correctly classified varied between 76% (32 of the 42 samples) and 93% (39 of the 42 samples) as seen in FIG. 51. FIG. 51 shows the success rate of an SVM linear classifier as a function of number of lipid peaks. In this analysis, the rodent data are used for model building, and the success rate is the percentage of rodents correctly classified in a leave-one-out procedure. Also in this analysis, the human data are used as a test set, and the success rate is the percentage of humans correctly classified by the rodent model. Further investigation of the classification and peak reduction procedures may lead to the confirmation that the diseased rodent model is a good model for metabolic disease in humans.

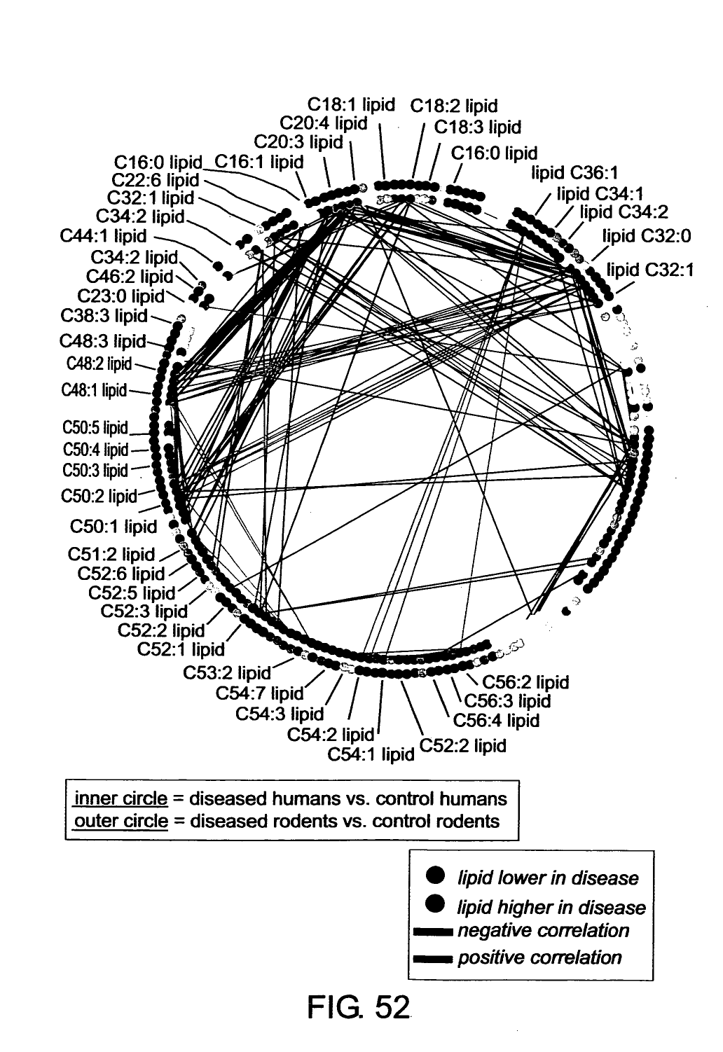

Results and discussion for the comparison of rodent samples with human samples—Common components. A comparison of the 571 LC/MS lipid peaks that were common to both species revealed that there were significant mean differences in both species between the diseased and control groups (at a significance level of 0.05 and using two-tailed pairwise t-tests) for 195 out of the 571 lipid LC/MS peaks. Of these 195 peaks, 185 exhibited the same trend in both species (higher or lower serum abundance in diseased vs. control). In addition, a number of correlations between pairs of lipid peaks were present both in the human and rodent samples, using an absolute value of Pearson correlation coefficient greater than 0.7, indicating that not only were the abundance differences conserved, but also that underlying mechanisms involved in the regulation of those lipid levels may likely be conserved across species. An excerpt of the results are summarized in FIG. 52.

More specifically, FIG. 52 shows comparison of lipid abundance changes and correlations across human and rodent species. In the figure, the large circles consist of elements, each of which representing a different LC/MS lipid peak. The shading of the elements corresponds to the relative abundance of the lipid in diseased vs. control samples. The relative abundances are normalized group mean differences. There are 195 such elements, all representing lipids with p<0.05. The outer large circle represents the diseased rodent vs. control rodent group comparison, while the inner concentric circle represents the diseased human vs. control human group comparison. The lines connecting pairs of elements in the figure are correlations, of Pearson coefficient |Cᵢⱼ|>0.70, which are present in both species.

Summary and conclusions. Metabolite and protein analyses of blinded serum samples from animal and human subjects were performed which allowed grouping of the samples based on their serum metabolite and protein profiles. Groups identified using clustering analysis reflected with 100% accuracy the phenotypic categories of the animal subjects and with a high degree of accuracy (>80%) the human subjects. Subsequent analyses identified many of the molecular components that differentiate the subjects.

These independent measures are informative in themselves. Moreover, when linked using correlation networks, one begins to see details of the biochemical processes that underlie the disease or drug response. One of the more interesting results is that the molecular components that differentiate the diseased rodents from the control rodents are very similar to those that differentiate the diseased humans from the control subjects. The wealth of data generated by this study illustrates the strengths of the Systems Biology approach utilizing an integrated platform of proteomics, metabolomics and informatics technologies.

Nomenclature/Terms Used in this Example

Abbreviations and Terms

| | |
|---|---|
| COSA: | Clustering Objects on Subsets of Attributes |
| CPMG NMR: | Carr-Purcell-Meiboom-Gill spin echo NMR |
| DE NMR: | Diffusion-edited NMR |
| LC: | Liquid Chromatography |
| MS/MS: | Tandem Mass Spectrometry |
| MS: | Mass Spectrometry |
| NMR: | Nuclear Magnetic Resonance |

Protein Nomenclature

Shotgun sequencing: a method of obtaining peptide sequence information using tandem mass spectra (MS/MS) acquired in a “data-dependent” instrument mode whereby the instrument is configured to measure MS/MS spectra for as many peptide peaks as possible. In this mode, the instrument runs a repeating scan cycle that consists of an initial survey scan of peptide peak signals to select the three or four that are most intense and subsequent MS/MS scans for each of the selected peaks.

Targeted sequencing: a method of obtaining peptide sequence information using tandem mass spectra (MS/MS) that were acquired for specified peptide peaks.

### EXAMPLE 6

### Systems Biology Approach: Human Cardiovascular Disease

The goal in this Example was to elucidate plasma metabolites that differentiate human cardiovascular disease patients from healthy subjects. In advance of the study, the subject samples were classified into either diseased or control categories (plasma samples from cardiovascular disease and matched, control subjects). Several metabolomics platforms that use NMR, LC/MS, and GC/MS technologies and data preprocessing software were applied to the comparative study of 80 plasma samples. The metabolomics profiling platforms generate datasets containing hundreds of spectral peaks that were initially not identified. Instead, peaks of statistical significance were determined. These entities were flagged for identification, using databases, additional MS/MS data, and expert interpretation, in the second phase of the analysis. Univariate and multivariate statistical analyses of the metabolomics datasets revealed measured features that were significantly different between the two groups of study subjects. Prior to the initiation of the second phase of the project, further classification of the diseased subjects on the basis of a clinical index of disease severity was used and additional statistical analyses were performed if any measured features correlate with the severity of the cardiovascular disease in the diseased group. Numerous features showed significance in one or more analysis and was identified. Then, a correlation network was constructed to visualize statistical and biological relationships among the identified, significant metabolites.

Objective. The goal of this study was to identify biomarker molecules as molecular differences between plasma samples taken from cardiovascular disease patients and matched control subjects.

Study design. The study was executed in two phases.
Phase I: metabolomics platforms were employed to comparatively profile 80 plasma samples described as being from either male cardiovascular disease patients (40 samples, mean age 53.4 years) or age-matched controls subjects (40 samples, mean age 51.6 years). The analytical platforms were CPMG NMR, diffusion-edited NMR, GC/MS, Lipid LC/MS, and Amino acid/global LC/MS. Software algorithms were used to extract spectral and chromatographic peak information from the raw data. Additional preprocessing was preformed to align the peaks among the datasets from each platform (i.e., chromatographic retention time alignment for LC- and GC/MS) for comparative statistical analyses. The peaks remained unidentified until flagged for identification on the basis of statistical significance. Identification activities were initiated on peaks that had different levels of abundance between the two experimental groups.
Phase II: Prior to the initiation of the second phase of the project, further classification of the diseased subjects on the basis of the clinical index of disease severity was made and additional statistical analyses were performed to determine if any measured features correlated with the severity of the disease in the diseased group. Where possible, further identification information was obtained for features deemed significant. A correlation network was then constructed to visualize statistical and biological relationships among the identified, significant metabolites.

Summary of methods. A number of analytical methods were used that enable the comparative profiling of a wide range of metabolites. The samples were analyzed using several analytical methods, and statistics were performed on unidentified peaks. Listed and briefly described below were the methods that were used.
(i) CPMG NMR: enhanced NMR measurement of low molecular weight metabolites at concentrations greater than 100 μM (e.g., amino acids, amino acid metabolites, organic acids, sugars).
(ii) GC/MS: global method designed for profiling of a wide range of metabolites classes (e.g., alcohols, aldehydes and cyclohexanols, amino acids, acyl amino acids, succinylamino acids, amines, aromatic compounds, fatty acids (greater than C6), organic acids, phospho-organic acids, sugars, sugar acids, sugar amines, sugar phosphates).
(iii) Lipids LC/MS: optimized for profiling of lipids and non-polar metabolites (e.g., lysophospholipids, phospholipids, cholesterol esters, diacylglycerols, triacylglycerols)
(iv) Amino acids/global LC/MS: optimized for profiling of amino acids and polar metabolites. Due to the presence of citrate, used as a blood anticoagulant, this platform did not yield usable data and was not used in Phase II.
(v) Diffusion-edited NMR: enhanced measurement of lipoprotein-associated metabolites. The profiled peaks are composites of signals from many lipid moieties and are therefore non-specific. Since uniquely identified molecular entities were preferred as biomarkers, this method was not pursued in Phase II.

Each of the above analyses yielded raw datasets that contain hundreds to thousands of peaks per sample. In order to enable comparative analysis of metabolite peak information across the entire sample set, several algorithms were applied to each raw data file for peak detection and signal integration. Next, to compensate for minor shifts in peak position that may occur in terms of retention time for LC/MS and GC/MS techniques or minor differences in chemical shift for the NMR techniques, algorithms were used to “align” the peaks. As a result of this process, each metabolite peak within a profile was assigned a peak identification number (or index number). This same identification number was used to describe the analogous peak found in the profiles from all other samples and therefore enabled comparative analyses of the integrated peak intensities.

Following univariate and multivariate statistical analyses of the data from each platform, metabolites that differentiated the diseased and healthy subjects were listed for identification in Phase II as ranked by the applied statistics.

Univariate results. Subsequent to data alignment and normalization, univariate homoscedastic t-tests with controls for false discovery rates were performed on identified metabolite analytes from all bioanalytical platforms used in the present study. Results showed twenty-four analytes which have adjusted p-values less than 0.05 based on a 10% false discovery control using the Benjamini-Hochberg approach.

Multivariate results. A multianalyte approach to finding sets of spectral peaks capable of categorizing diseased samples and control samples was also pursued. In the literature, this problem of finding a biomarker composed of more than one molecular component able to segregate groups of samples is referred to as a ‘classification problem.’ In the present case, only those analytes which had been confidently and uniquely identified were used; there were ninety-four such analytes at the time of the analysis. This number does not include isotopes, adducts, redundant ¹NMR resonance peaks, and the like, which also may have been identified. The challenge of classification, in brief, is to determine a multianalyte biomarker composed of the minimal number of most informative analytes.

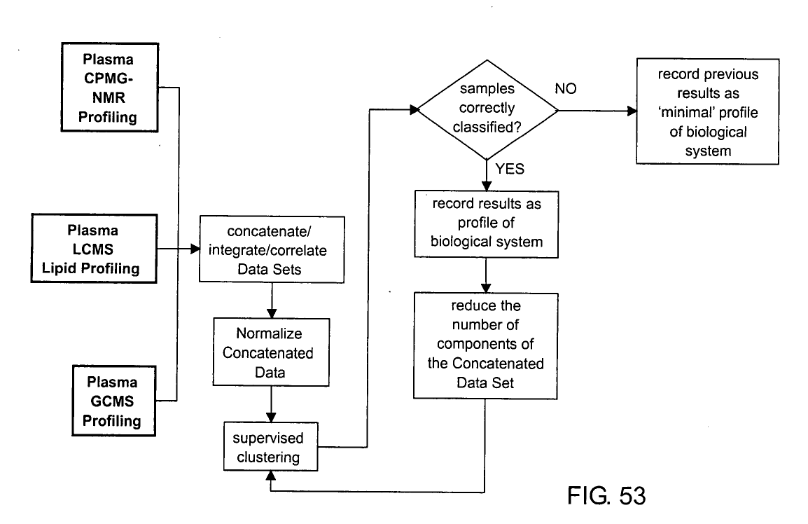

In considering biomarkers composed of more than one component, a number of points were considered. These include determining which subset of analytes is the optimal one to include in the marker; how well the final biomarker performs in correctly classifying the sample set at hand; and how well the final biomarker performs in correctly classifying samples from an independent sample set. In addition to the above items, the biochemical relevance of the components constituting the biomarker is also important, as is the feasibility of developing a practical diagnostic assay for the final biomarker. With the latter in mind, the minimal optimal number of analytes which will achieve the best predictive performance criteria was determined. FIG. 53 depicts the outline of the steps of this analysis. In general, multiple data sets from multiple analytical platforms are concatenated, integrated, and correlated, and then are normalized. This data is further analyzed through a supervised clustering analysis to obtain a profile of a biological system. A brief overview of the methodology of constructing a multianalyte biomarker is presented below.

In order to determine the minimal optimal subset of spectral peaks which best segregate disease and control samples, an approach known as Recursive Feature Elimination is used. This approach proceeds as follows.
1. Choose a ‘classification algorithm’ which accepts as input N components (i.e., N spectral peaks), and returns (i) the success of segregating control and disease samples (as measured by specificity and sensitivity) achieved by a linear combination of the N components, and (ii) a ranking of the N input components based on their contribution to the classification.
2. Allow all analytes (aligned, normalized and pre-processed) as input to the classification algorithm.
3. With these components as inputs, run the algorithm to converge upon a linear combination of input analytes to be used to classify control and disease samples.
4. Record the ranking criterion (‘weight’) for each analytes. The weights are the coefficients in the linear combination of input components as determined by the algorithm (the final weight is actually a mean weight, averaged over multiple Cross-Validation iterations).
5. Compute the ‘Cross-Validation’ performance of this combination of spectral peaks in classifying control and disease samples using the Cross-Validation method (discussed below), as well as the standard error for the cross-validation tests.
6. Remove the analyte with the lowest weight.
7. Repeat Step 3 through Step 6, until only one analyte remains.
8. Determine the minimum number of analytes required to achieve the highest success in segregating control and disease samples; this biomarker is composed of a linear combination of analyte values, the coefficients in the combination being the weights corresponding to each analyte.
   The term ‘Recursive Feature Elimination’ reflects the successive pruning of the list of spectral peaks by one spectral peak for each iteration of Steps 3 through 6.

In the present study, one classification algorithm was applied. This algorithm involves a state-of-the-art approach referred to as a ‘Logistic Classifier’ (Anderson, 1982). This method has its origins in handwriting and biometric pattern recognition. It is designed to select for a final biomarker comprising components with low mutual correlation, a desirable trait to avoid redundancy and minimize biomarker size. While the general principles of the technique are known, the current analysis optimizes it to work with data derived from the particular bioanalytical profiling platforms discussed earlier.

There are two different tests of performance which have been applied for the processes outlined in this section.
1. ‘Cross-Validation Performance’ is the classification success of a biomarker which has been constructed based on a subset of the available samples, and tested on the remaining samples which have been a priori intentionally left out (Hastie, 2001). A typical situation for the present study is to construct a biomarker based only on thirty-two (34) diseased samples and thirty-two (34) control samples chosen at random, and to test the performance (classification success) of the resultant biomarker in classifying the remaining six (6) diseased and six (6) control samples which were excluded. This process is repeated successively many times, with different sets of randomly chosen 6+6 samples ‘left out’. The reported ‘Cross-Validation Performance’ for the biomarker is the averaged performance of many such permutations; typically ten cross-validation rounds are used.

It is important to note that the purpose of Cross-Validation is to assess the generalizability of a biomarker, within the limitations posed by the availability of a relatively limited number of independent samples. In the absence of independent samples from a different population of patients, the Cross-Validation Performance is an estimation of the performance of the biomarker on an independent test set of samples. Such an extrapolation is made possible by measuring the performance of the biomarker on the many permutations and combinations of subsets of the available samples; this process effectively simulates a situation in which many more samples are available.
2. ‘Permutation Performance’ is the performance of the multivariate biomarker selection algorithm when sample labels have been randomly permuted. This occurs over may such random permutations, and the average performance is reported. A robust classifier—one which is not overfit to the training set—should yield a permutation performance of approximately 50% (i.e., chance performance).

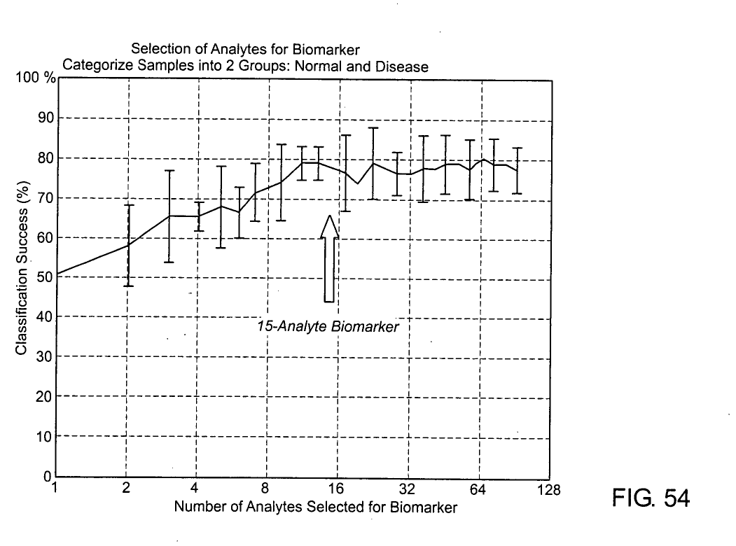

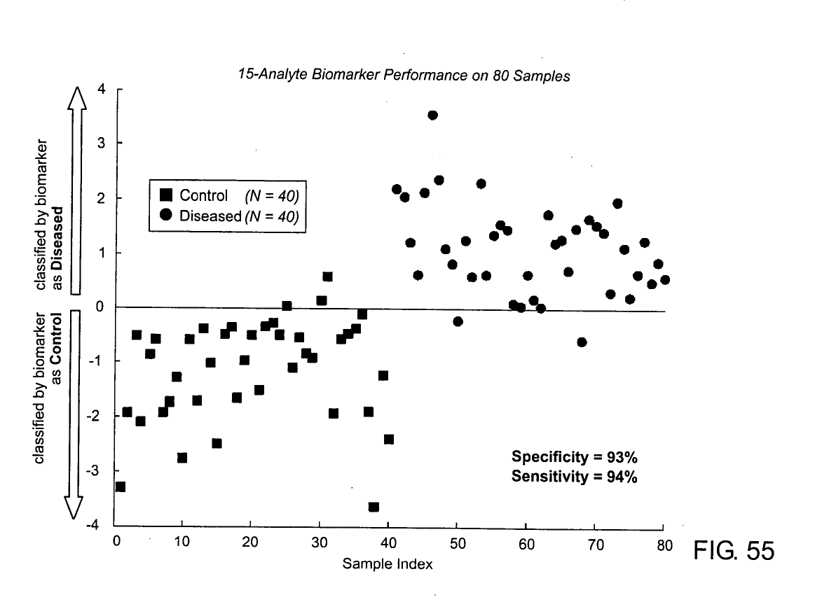

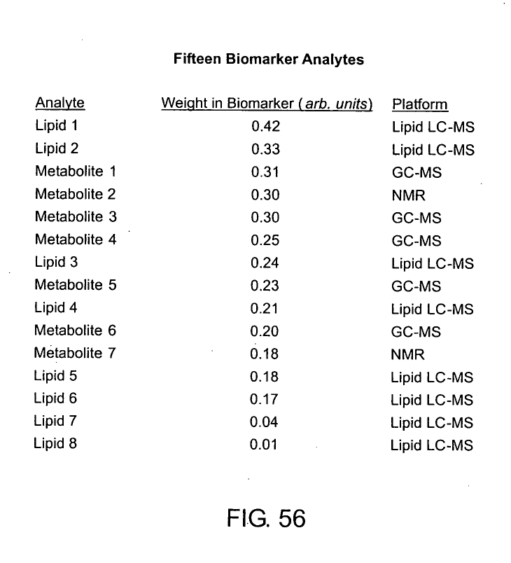

Results and discussion. The results of these classification methods are graphically shown in FIG. 54. A biomarker set of fifteen molecular components was identified as part of a profile the human cardiovascular disease. These molecular components of the biomarker set were discovered by using multivariate statistical analysis methods and integration of a plurality of datasets including those for more than one type of measurement technique and those for more than one biomolecular component type as shown in FIG. 56. This methodological approach was used successfully to generate a biomarker set which could classify the 80 samples. FIG. 55 shows the classification of each subject as a disease or control group member using these biomarkers. A sensitivity of 93% and a specificity of 94% were obtained.

The abbreviations used in this example are, where appropriate, the same as those used in Example 5.

Each of the patent documents and scientific publications disclosed hereinabove is incorporated by reference herein for all purposes.

Although the invention has been particularly shown and described with reference to specific embodiments, it should be understood by those skilled in the art that various changes in form and detail may be made therein without departing from the spirit, essential characteristics or scope of the invention. The foregoing embodiments are therefore to be considered in all respects illustrative rather than limiting on the invention described herein. The scope of the invention is thus indicated by the appended claims rather than by the foregoing description, and all changes which come within the meaning and range of equivalency of the claims are therefore intended to be embraced therein.

1. A method of profiling a state of a biological system in a mammal, the method comprising the steps of:
(a) evaluating with statistical analysis a plurality of data sets of a biological system and comparing features among the plurality of data sets to determine one or more sets of differences among at least a portion of the plurality of data sets; and
(b) developing a profile for a state of the biological system based on the results of step (a),
wherein the plurality of data sets comprise measurements derived from more than one biological sample type, more than one type of measurement technique, more than one biomolecular component type, or a combination of at least two of a biological sample type, a measurement technique, and a biomolecular component type.

2. The method of claim 1 wherein the biological system is in a human.

3. The method of claim 1 wherein the statistical analysis comprises multivariate analysis.

4. The method of claim 1 wherein the biological sample type is selected from the group comprising blood, plasma, serum, cerebrospinal fluid, bile, saliva, synovial fluid, pleural fluid, pericardial fluid, peritoneal fluid, sweat, feces, nasal fluid, ocular fluid, intracellular fluid, intercellular fluid, lymph, urine, liver cells, epithelial cells, endothelial cells, kidney cells, prostate cells, blood cells, lung cells, brain cells, skin cells, adipose cells, tumor cells, and mammary cells.

5. The method of claim 1 wherein a plurality of data sets are derived from one biological sample type that is treated differently, or from one biological sample type that is collected or analyzed at different times.

6. The method of claim 1 wherein the measurement technique is selected from the group comprising liquid chromatography, gas chromatography, high performance liquid chromatography, capillary electrophoresis, mass spectrometry, liquid chromatography-mass spectrometry, gas chromatography-mass spectrometry, high performance liquid chromatography-mass spectrometry, capillary electrophoresis-mass spectrometry, nuclear magnetic resonance spectrometry, parallel hybridization assay, parallel sandwich assay, and competitive assay.

7. The method of claim 1 wherein a plurality of data sets comprise measurements from different instrument configurations of a single type of measurement technique.

8. The method of claim 1 wherein the biomolecular component type is a gene, a gene transcript, a protein, or a metabolite.

9. The method of claim 1 comprising the step of comparing the profile for a state of the biological system to a database of profiles.

10. The method of claim 1 comprising comparing the profile for a state of the biological system to a profile of another state of a biological system.

11. An article of manufacture having a computer-readable medium with computer-readable instructions embodied thereon for performing the method of claim 1.

12. A method of profiling a state of a biological system in a mammal, the method comprising the steps of:
(a) evaluating with statistical analysis a plurality of data sets for a biomolecular component type and comparing features among the plurality of data sets to determine one or more sets of differences among at least a portion of the plurality of data sets;
(b) evaluating with statistical analysis a plurality of data sets for another biomolecular component type and comparing features among the plurality of data sets to determine one or more sets of differences among at least a portion of the plurality of data sets; and
(c) correlating the results of step (a) and step (b) to develop a profile for a state of the biological system.

13. The method of claim 12 wherein the plurality of data sets for a biomolecular component type or another biomolecular component type comprise measurements derived from more than one biological sample type, more than one type of measurement technique, or a combination of a biological sample type and a measurement technique.

14. The method of claim 12 wherein the biomolecular component type is a protein and the other biomolecular component type is a metabolite.

15. The method of claim 12 wherein the biomolecular component type is a gene transcript and the other biomolecular component type is a metabolite.

16. A method of profiling a state of a biological system in a mammal, the method comprising the steps of:
(a) evaluating with statistical analysis a plurality of data sets comprising measurements from at least two biomolecular component types and comparing features among the plurality of data sets to determine one or more sets of differences among at least a portion of the plurality of data sets; and
(b) developing a profile for a state of the biological system based on the results of step (a).

17. The method of claim 16 wherein the plurality of data sets comprise measurements derived from more than one biological sample type, more than one type of measurement technique, or a combination of a biological sample type and a measurement technique.

18. The method of claim 16 wherein the step of evaluating comprises:
evaluating a plurality of data sets for a biomolecular component type and comparing features among the plurality of data sets to determine one or more sets of differences among at least a portion of the plurality of data sets; and
evaluating a plurality of data sets for another biomolecular component type and comparing features among the plurality of data sets to determine one or more sets of differences among at least a portion of the plurality of data sets.

19. The method of claim 16 wherein the at least two biomolecular component types comprise a protein and a metabolite.

20. The method of claim 16 wherein the at least two biomolecular component types comprise a gene transcript and a metabolite.
